SEQUENCE LISTING (6 sequences)

SEQ ID NO: 1 - DNA, 19 bp, Artificial
  Primer
aatgggtttgaaagggcca

SEQ ID NO: 2 - DNA, 20 bp, Artificial
  Primer
tcaagacctgctcttccgct

SEQ ID NO: 3 - DNA, 32 bp, Artificial
  Primer
aacctggaaatcaaagattgggaactagtgga

SEQ ID NO: 4 - DNA, 19 bp, Artificial
  Primer
cacgctgagatgccttgct

SEQ ID NO: 5 - DNA, 23 bp, Artificial
  Primer
accatcttctctgagctcctcaa

SEQ ID NO: 6 - DNA, 29 bp, Artificial
  Primer
cagcccatggcctacaactgtgggttata

(19) United States
(12) Patent Application Publication
(10) Pub. No.: US 2010/0119474 A1
(43) Pub. Date: May 13, 2010

(54) CHRONIC OBSTRUCTIVE PULMONARY DISEASE SUSCEPTIBILITY AND RELATED COMPOSITIONS AND METHODS

(75) Inventors: Ronald G. Crystal, New York, NY (US); Neil R. Hackett, New York, NY (US); Adriana Heguy, Forest Hills, NY (US); Ben-Gary Harvey, West Hempstead, NY (US); Philip L. Leopold, Basking Ridge, NJ (US); Ann E. Tilley, New York, NY (US); Timothy O'Connor, New York, NY (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Cornell University, Ithaca, NY (US)

(21) Appl. No.: 12/529,838

(22) PCT Filed: Mar. 6, 2008
(86) PCT No.: PCT/US08/56091

§ 371 Date: Nov. 3, 2009

Related U.S. Application Data
(60) Provisional application No. 60/893,283, filed on Mar. 6, 2007.

(51) Int. Cl. A61K 38/20; G01N 33/53; A61K 38/45; A61K 38/47; A61K 38/44; A61K 38/21; A61P 11/00

(52) U.S. Cl. 424/85.2; 435/7.21; 424/94.5; 424/94.61; 424/94.4; 424/85.5

(57) ABSTRACT

The invention provides a method of determining the likelihood that a smoker will or will not develop chronic obstructive pulmonary disease (COPD) by obtaining a sample from the smoker, analyzing the sample for the expression of a set of biomarkers associated with COPD, and comparing the expression pattern determined in the sample with a standard expression pattern to determine the likelihood that the smoker will or will not develop COPD. The invention further provides a composition, a method of treatment, and methods of determining the efficacy of treatment for COPD.

### CROSS-REFERENCE TO RELATED APPLICATIONS

This patent application claims the benefit of U.S. Provisional Patent Application No. 60/893,283, filed Mar. 6, 2007, which is incorporated by reference.

### STATEMENT REGARDING FEDERALLY SPONSORED RESEARCH AND DEVELOPMENT

This invention was made in part with Government support under Grant Numbers 1 RO1 HL074326-03 and P50 HL084936 awarded by the NIH/NHLBI, as well as MO1 R000047 awarded by the NIH. The Government may have certain rights in this invention.

### BACKGROUND OF THE INVENTION

Chronic obstructive pulmonary disease (COPD) is a group of diseases characterized by limitation of airflow in the airway that is not fully reversible. COPD is the umbrella term for chronic bronchitis and/or emphysema. The leading cause of COPD is smoking. Continuous smokers have at least a 15-25% risk of developing COPD.

While smokers are more likely than nonsmokers to develop COPD, there are long term heavy smokers who never develop the disease whereas there are light smokers who do develop COPD. The difference in smokers who develop COPD and those who do not likely is genetically related. There is currently no way to determine which smokers will or will not develop COPD.

Accordingly, a method of determining the likelihood that a smoker will or will not develop COPD would be desirable. Such a method would be particularly useful and would provide an opportunity for prophylactic treatment to prevent or at least delay the onset of COPD.

### BRIEF SUMMARY OF THE INVENTION

The invention provides a method of determining the likelihood that a smoker will or will not develop COPD. The method comprises (a) providing a sample obtained from a smoker, (b) analyzing the sample to determine the expression pattern of one or more biomarkers associated with COPD, and (c) comparing the expression pattern determined from the sample with a standard expression pattern to determine the likelihood that the smoker will or will not develop COPD.

The invention also provides a composition comprising (a) a pharmaceutically acceptable carrier and (b) a substance which causes an expression pattern of one or more biomarkers associated with COPD that is indicative of acquiring COPD to be more similar to an expression pattern of one or more biomarkers associated with COPD that is indicative of not acquiring COPD.

The invention further provides a method to determine the efficacy of treatment for COPD. The method comprises (a) providing a sample obtained from a subject that is undergoing treatment for COPD, (b) analyzing the sample to determine the expression pattern of one or more biomarkers associated with COPD, and (c) comparing the expression pattern determined from the sample with a standard expression pattern or an expression pattern obtained from a sample obtained from the subject at an earlier time to determine whether the treatment for COPD has or has not been effective.

### BRIEF DESCRIPTION OF THE SEVERAL VIEWS OF THE DRAWING(S)

FIG. 1 is a table setting forth probes (Probe No. and Probe Set ID) utilized in the context of the invention, as well as the corresponding gene (Gene Symbol and/or Gene Name/Description, as available), the ratio of the mRNA levels in samples obtained from smokers to the mRNA levels in samples obtained from non-smokers with respect to each probe (SINS), and an indication of the significance of the ratio in terms of a p value for the ratio (p(BH)).

FIG. 2 is a schematic of the functional groups of genes expressed in the lung of a normal nonsmoker.

FIG. 3A is a graph of the relative expression of IL4R, SPON2, SUSD4, and CX3CL1 genes in smokers versus nonsmokers.

FIG. 3B is a graph of the relative expression of HTATIP2, PIR, GADD45B, and HIPK2 genes in smokers versus nonsmokers.

FIG. 3C is a graph of the relative expression of CYP1B1, AKR1C1, AKR1C2, and GPX2 genes in smokers versus nonsmokers.

FIG. 3D is a graph of the relative expression of ATP6V0A4, CCHCR1, FOXA2, and FZD8 genes in smokers versus nonsmokers.

FIG. 4 is a graph of the normalized expression level of NQ01, ALDH3A1, AKR1C3, ADH7, PIR, HIPK2, CDKN1C, FOXA2, and CX3CL1 in smokers and nonsmokers as assessed by microarray and TAQMAN RT-PCR.

FIG. 5A is a graph of the relative expression level of pirin in smokers versus nonsmokers.

FIG. 5B is a graph of the relative expression level of pirin in nonsmokers and smokers as assessed by microarray and TAQMAN RT-PCR.

FIG. 6 is a graph of relative pirin RNA expression level in control human bronchial epithelial cells and in cells after 10% and 100% cigarette smoke exposure in vitro.

FIG. 7A is a graph of the relative pirin RNA expression level in BEAS-2B cells exposed to Adpirin versus BEAS-2B cells exposed to AdNull.

FIG. 7B is a graph of the relative pirin RNA expression level in BEAS-2B cells exposed to Adpirin versus BEAS-2B cells exposed to AdNull over time.

FIG. 8 is a graph of the percentage of apoptotic cells/high powered field of naïve BEAS-2B cells, and BEAS-2B cells exposed to AdPirin and AdNull.

FIG. 9A is a graph of the apoptotic index of BEAS-2B cells exposed to cigarette smoke (CSE), AdNull, and AdPirin.

FIG. 9B is graph of the relative pirin RNA levels in BEAS-2B cells exposed to cigarette smoke (CSE), AdNull, and AdPirin.

FIG. 10 is a graph of the normalized expression level osteopontin, ADAM10, and chemokine (C-X-C motif) ligand 6 in alveolar macrophages of smokers and nonsmokers as assessed by microarray and TAQMAN RT-PCR.

FIG. 11 is a graph of the normalized expression of ASCL1, SCG2, CHGA, ENO2, and GRP in the small airway epithelium of normal nonsmokers, normal smokers, smokers with early COPD, and smokers with established COPD.

FIG. 12A is a graph of the normalized expression of UCHL1 in the large airways of a normal nonsmoker and a normal smoker.

FIG. 12B is a graph of the normalized expression of UCHL1 in the large and small airways of a normal nonsmoker and a normal smoker.

FIG. 12C is a graph of the normalized expression of UCHL1 in the large and small airways of a normal nonsmoker, normal smoker, smoker with early COPD, and smoker with established COPD.

FIG. 13 is a graph of the average expression level of CHGA, GRP, ENO2, SCG2, and UCHL1 in the small airways of normal nonsmokers and normal smokers.

FIG. 14A is a graph (volcano plot) of differential gene expression profiles in the small airway epithelium in non-smokers and smokers. Expression levels normalized by array and by gene were compared for 41 healthy smokers and 34 healthy non-smokers for all probe sets “present” in at least 20% of samples (Affymetrix HG-U133 Plus 2.0 array). The mean expression level for each group provides the fold-change (abscissa) versus p value (ordinate) by t test. Each probe set is represented by a filled circle, with probe sets that are not significantly different in smokers compared to non-smokers in light gray and those that are significantly different in the two groups in dark gray. Probe sets with a higher expression level in smokers are to the top right, and probe sets with a lower expression level in smokers are to the top left.

FIG. 14B is a graph (skyscraper plot) of fold changes for 619 probe sets significantly differentially expressed in smokers versus non-smokers. Expression levels normalized by array and by gene were compared for 41 healthy smokers and 34 healthy non-smokers for all probe sets “present” in at least 20% of samples (Affymetrix HG-U133 Plus 2.0 array). Genes upregulated in smokers have fold changes>1; those downregulated in smokers have fold changes<1 on this log scale. Alternating gray and white bands highlight the probe sets belonging to specific functional categories.

FIG. 15 is a graph comparing the I_{SAE} of healthy non-smokers (n=34, white bars) and healthy smokers (n=41, gray bars). The I_{SAE} distinguishes most smokers from non-smokers, with a large range among smokers. The quartiles of smokers are indicated with dashed lines. The ISAE for healthy non-smokers has a median of 1.6%, a variance of 4.3%, and an inter-quartile range (IQR) of 0.8-3.2. The I_{SAE} for healthy smokers is significantly greater than that of non-smokers, with a median of 26.5%. The range among healthy smokers is much greater than that of non-smokers, with a variance of 140% and in IQR of 16.8-34.4%.

FIG. 16A is a frequency distribution of I_{SAE} among healthy non-smokers and healthy smokers.

FIG. 16B is a frequency distribution of I_{SAE} among smokers with COPD.

FIG. 17 is a graph of I_{SAE} values for nonsmokers (white bars), healthy smokers (hatched bars), and COPD smokers (black bars). 90% of COPD smokers have values within the third and fourth quartile of healthy smokers' values, and 75% of COPD smokers fall within the fourth quartile of healthy smokers' values.

### DETAILED DESCRIPTION OF THE INVENTION

The invention provides a method of determining the likelihood that a smoker will or will not develop COPD. The method comprises (a) providing a sample obtained from a smoker, (b) analyzing the sample to determine the expression pattern of one or more biomarkers associated with COPD, and (c) comparing the expression pattern determined from the sample with a standard expression pattern to determine the likelihood that the smoker will or will not develop COPD.

The sample to be analyzed can be any sample that contains biomarkers associated with COPD. The sample is desirably a tissue from the subject. Desirably, the sample is airway tissue (e.g., lung tissue) or nasal tissue. Suitable airway tissue can include tissue from the trachea, large airway, and small airway. Samples also may include sputum (i.e., mucus or phlegm) and cells obtained via pulmonary lavage. Alternatively, the sample may be blood. Cells contained in the sample may include epithelial cells (i.e., small airway epithelium, large airway epithelium, trachea airway epithelium, nasal epithelium, etc.), and inflammatory cells, such as alveolar macrophages. Samples of airway epithelium, especially small airway epithelium, have been demonstrated to be particularly useful in the context of the invention.

The sample may be obtained by any suitable method. For example, a lung tissue sample may be obtained by bronchoscopy utilizing a local anesthetic (such as xylocaine), under conscious sedation, etc. Samples may additionally be obtained via pulmonary lavage, expectoration by the subject, or via venous or arterial blood withdrawal.

The expression pattern of one or more biomarkers associated with COPD is determined in the inventive method. The expression pattern of any suitable number of biomarkers can be determined. For example, the expression pattern of 2 or more, 3 or more, 4 or more, 5 or more, 10 or more, 15 or more, 20 or more, 30 or more, 50 or more, 60 or more, 70 or more, 80 or more, 90 or more, 100 or more, 150 or more, 200 or more, 250 or more, 300 or more, 350 or more, or 400 or more biomarkers can be determined.

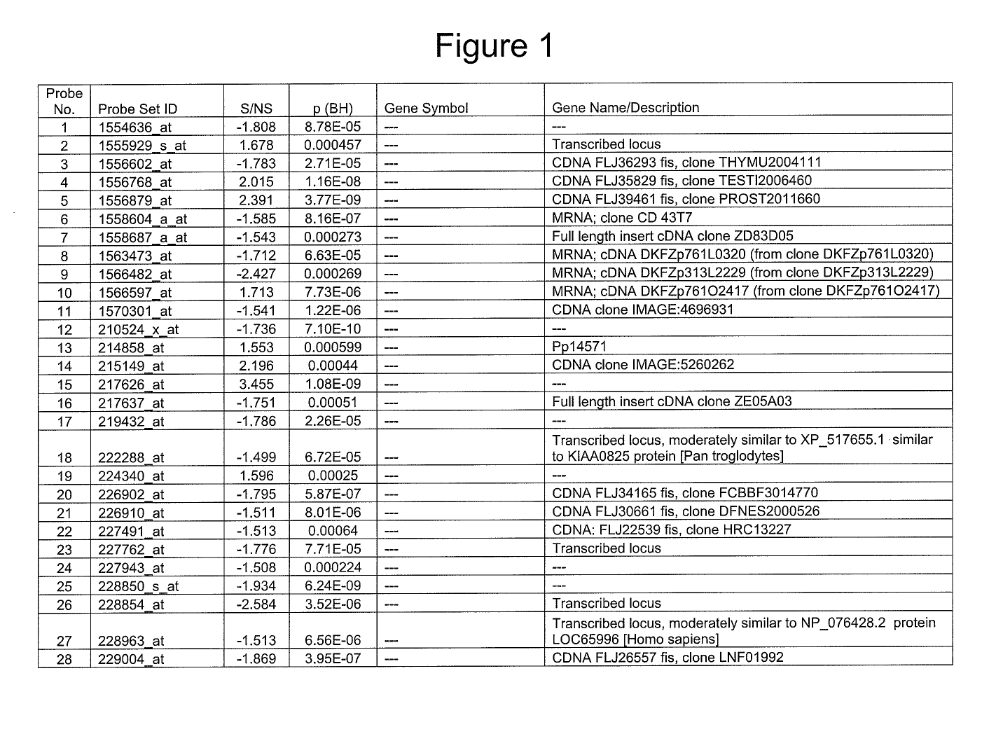

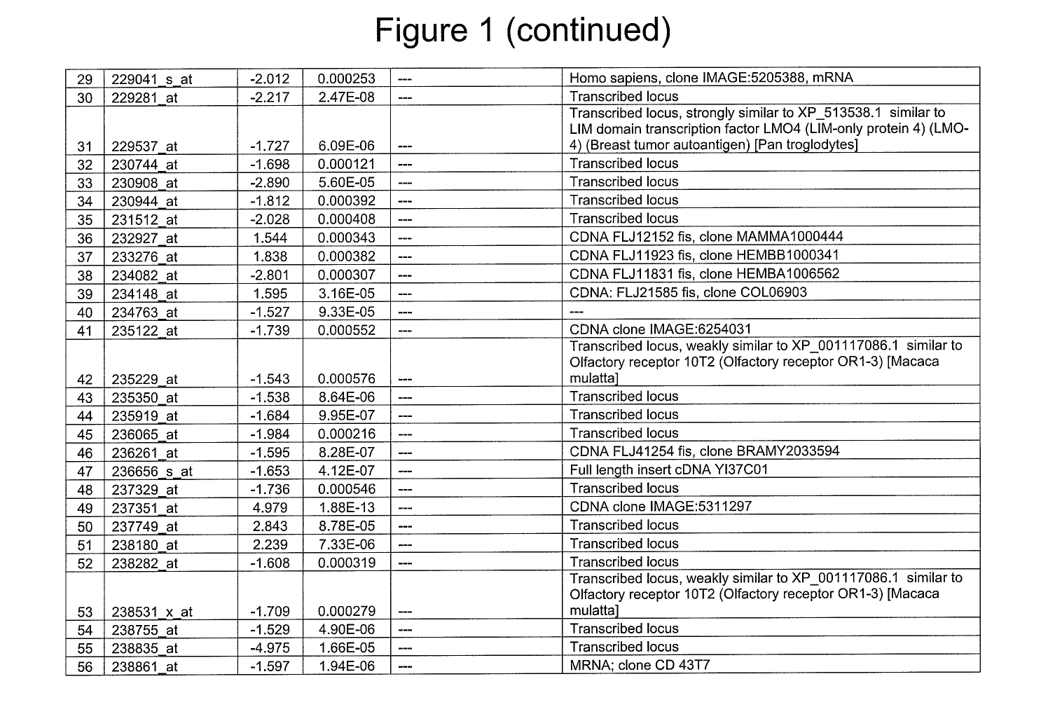

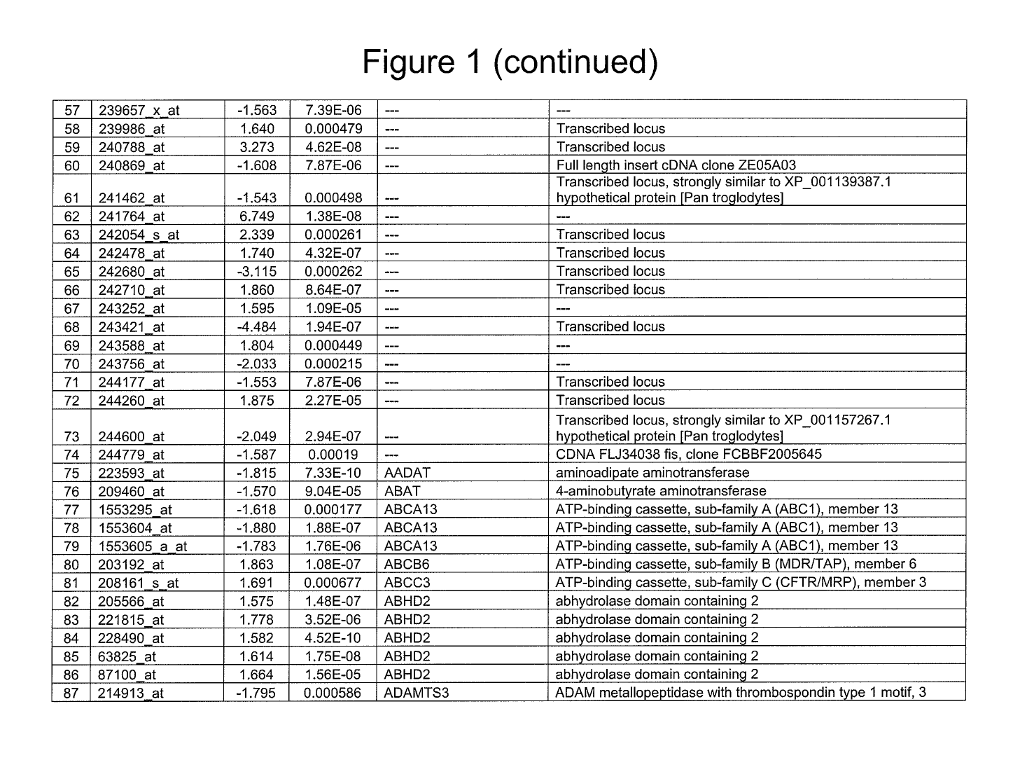

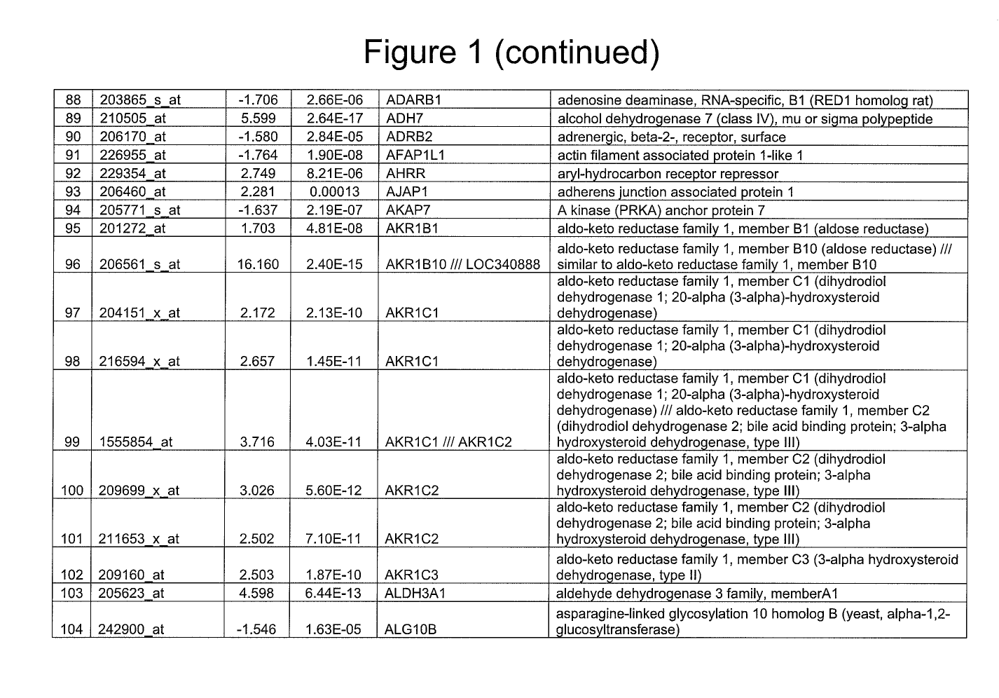

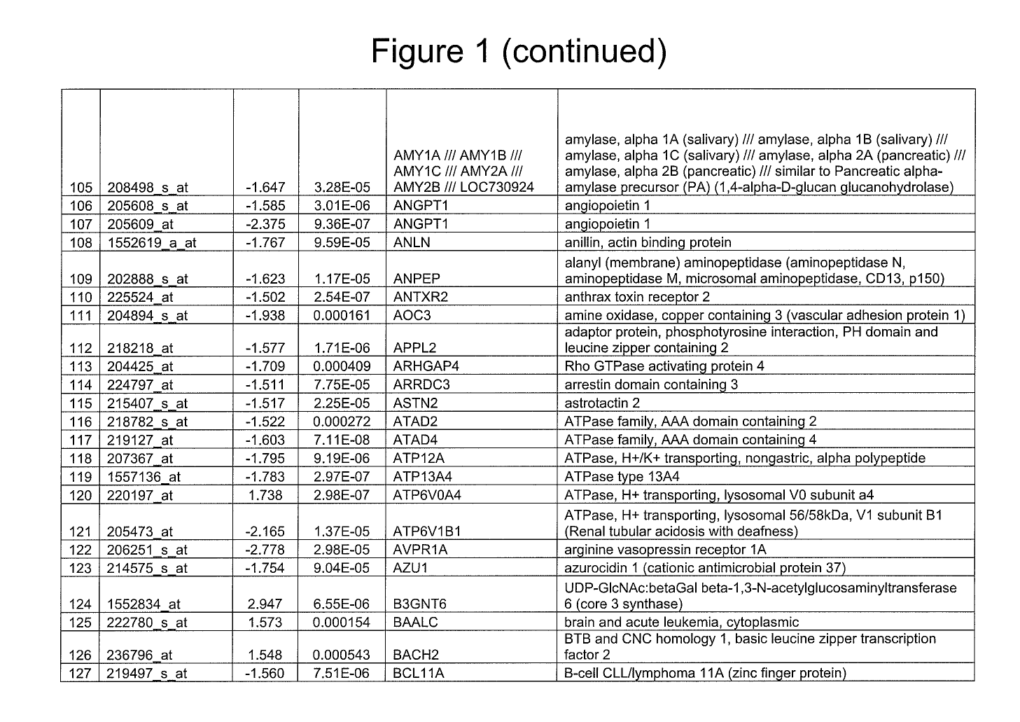

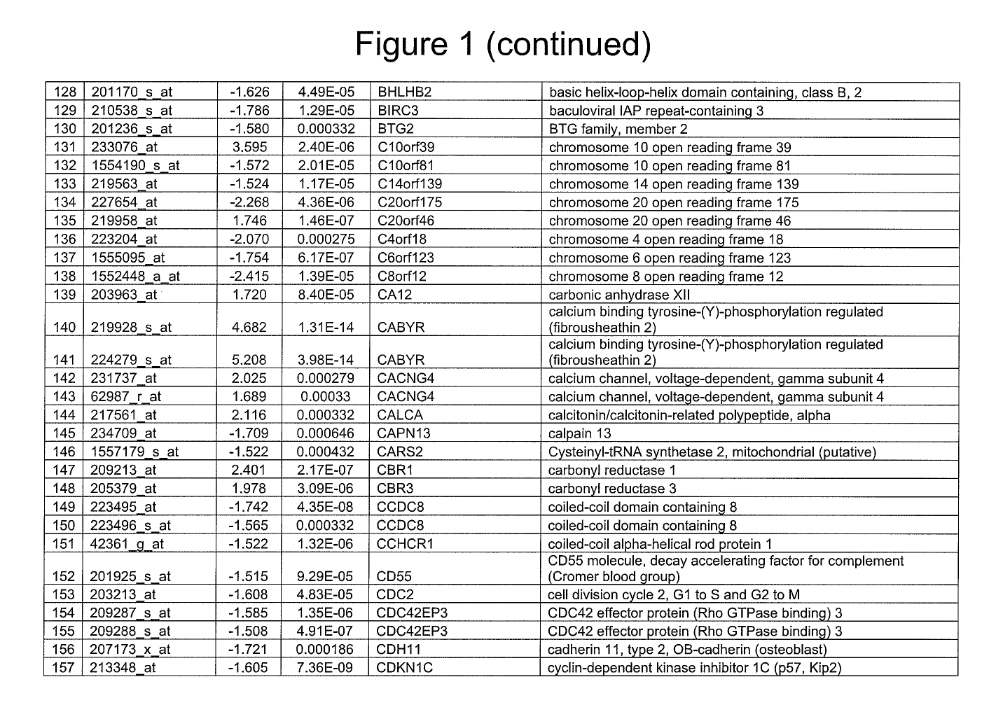

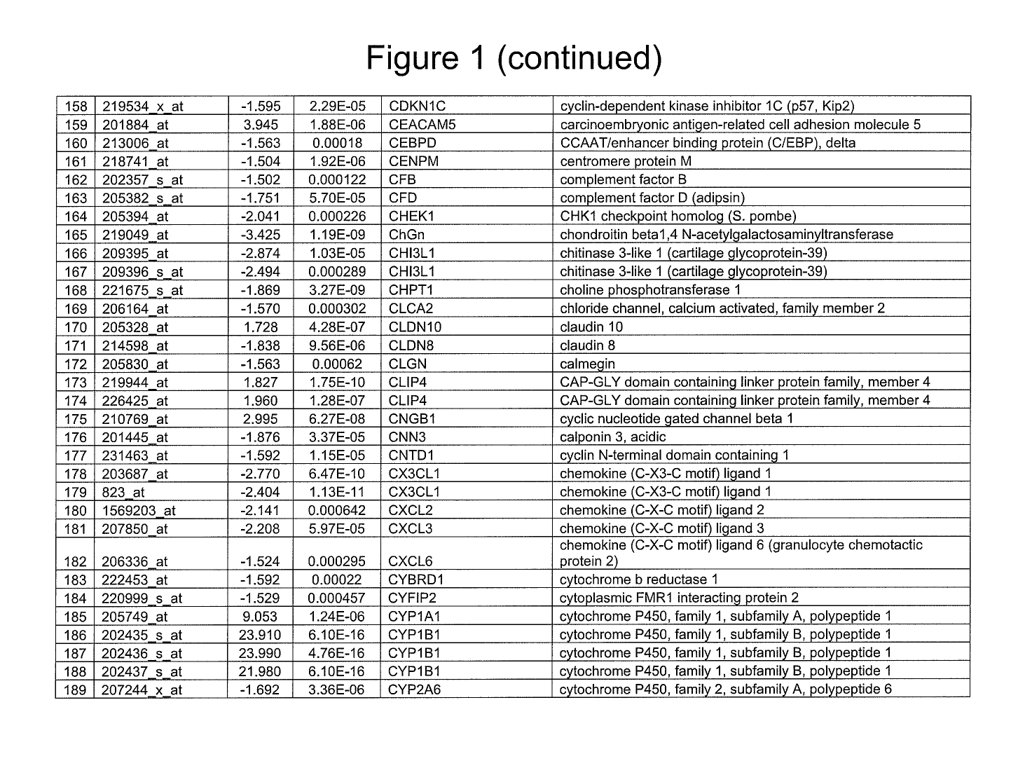

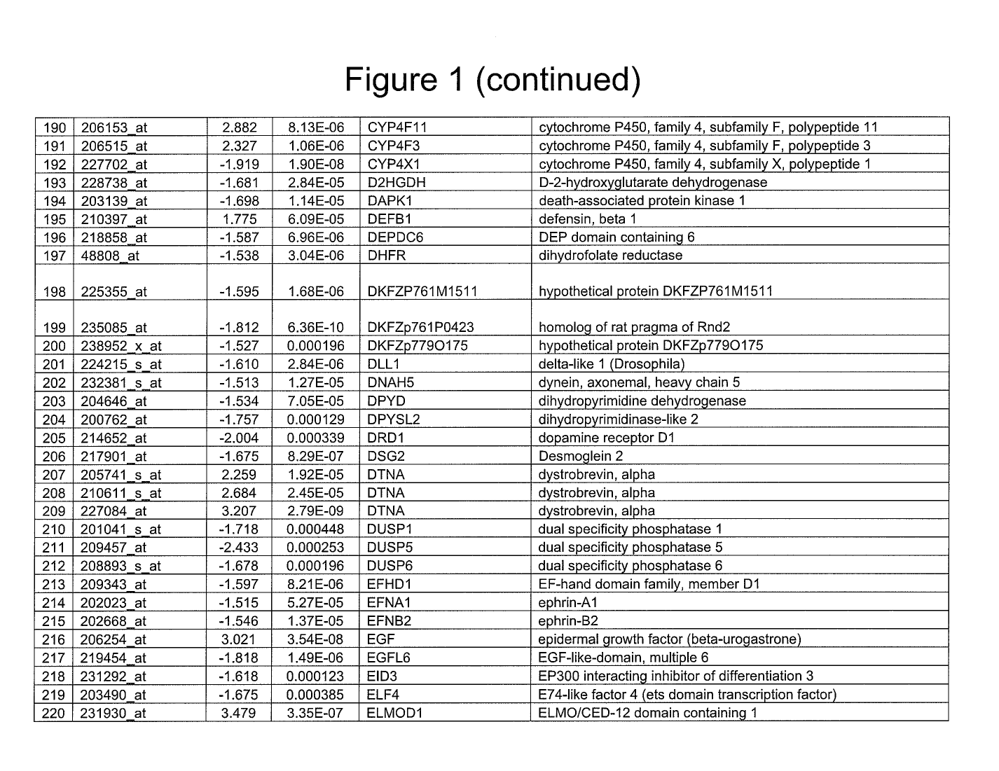

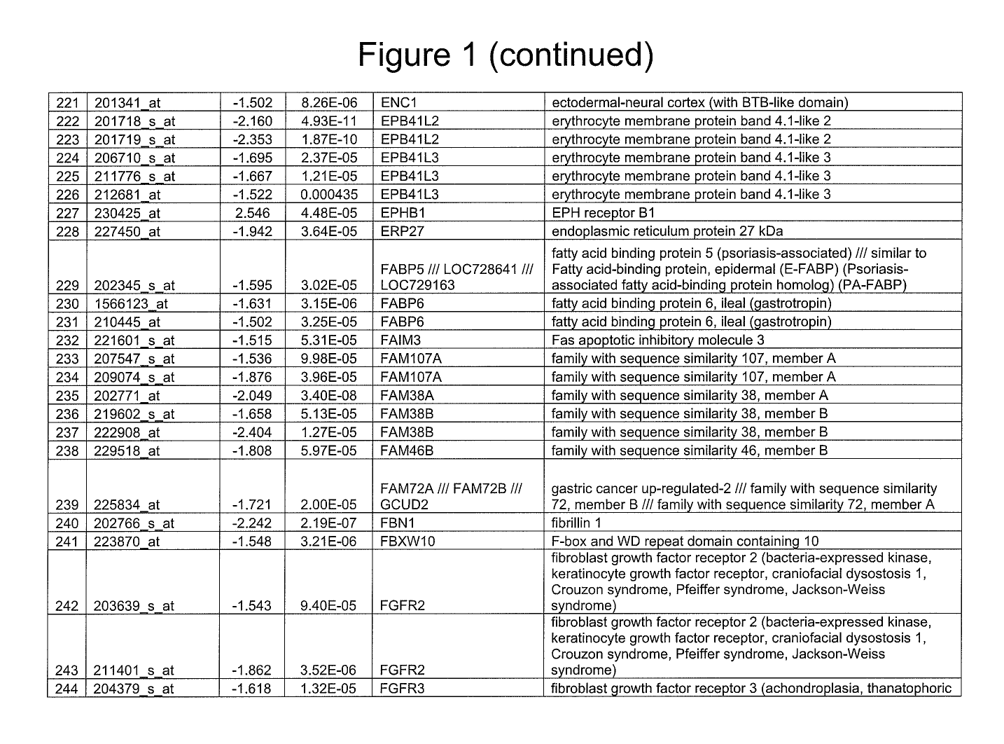

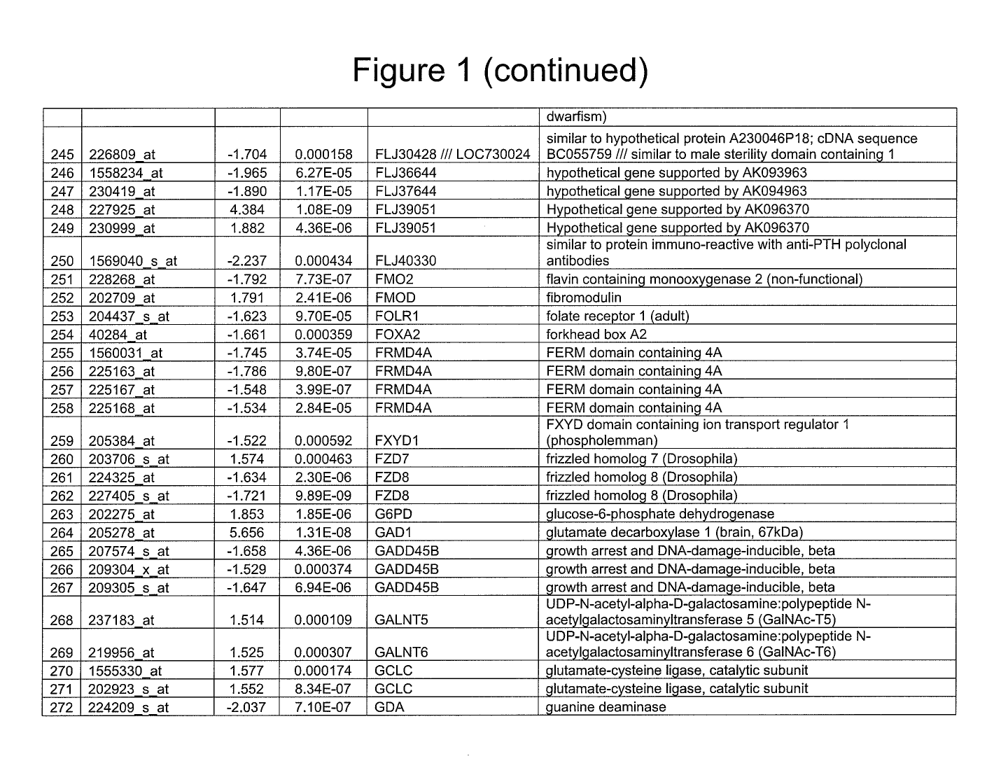

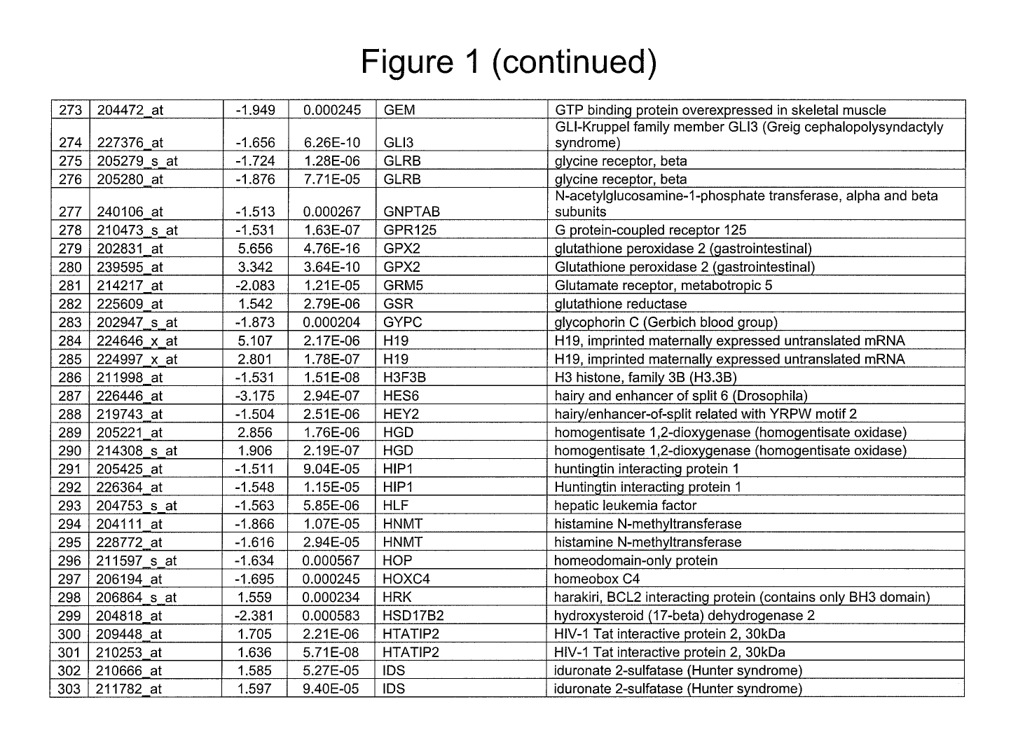

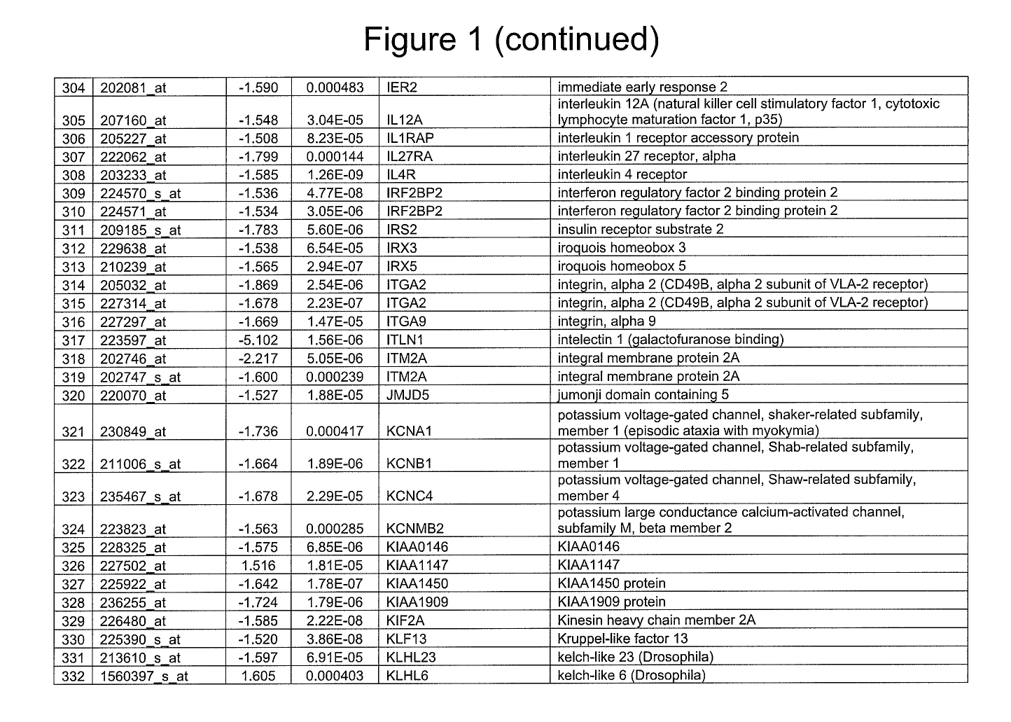

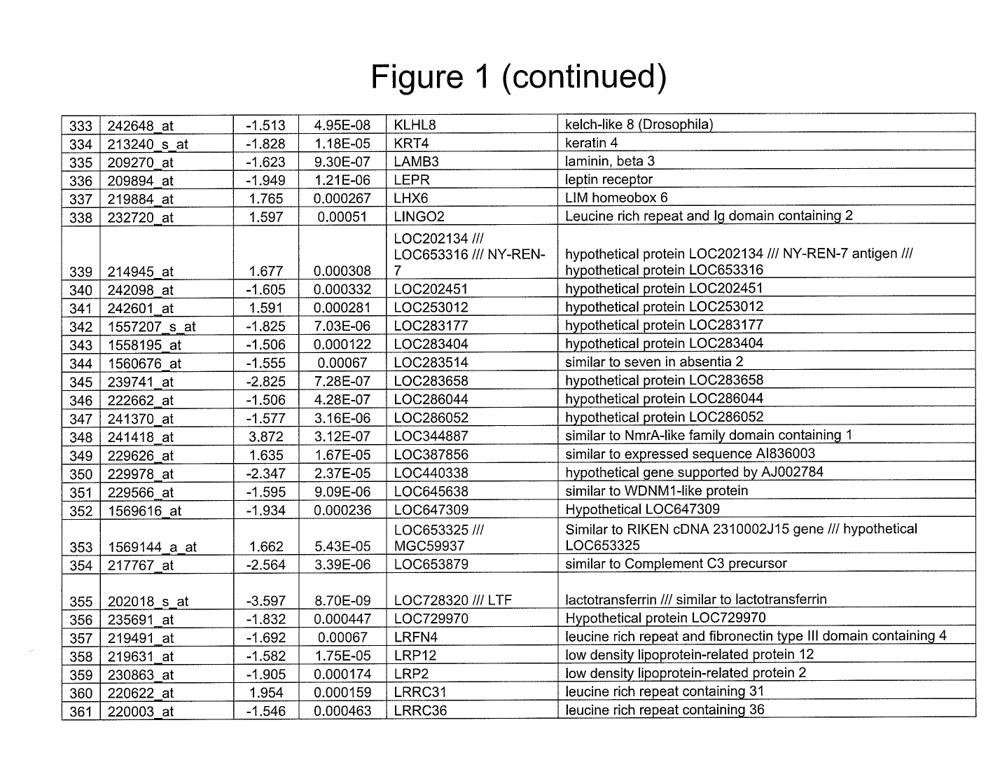

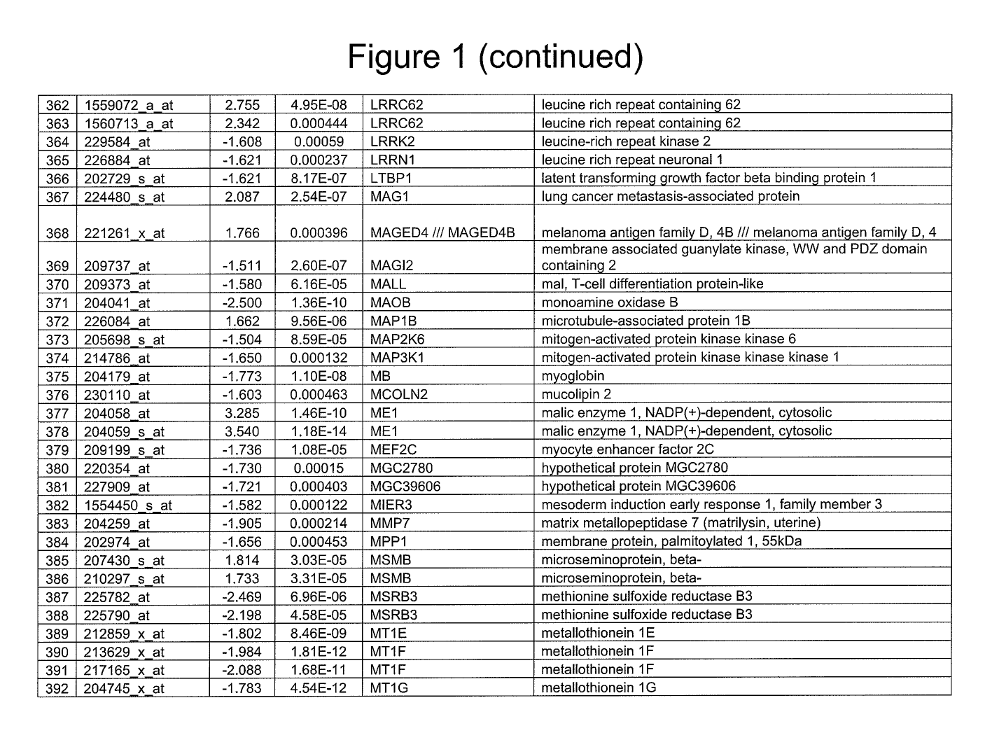

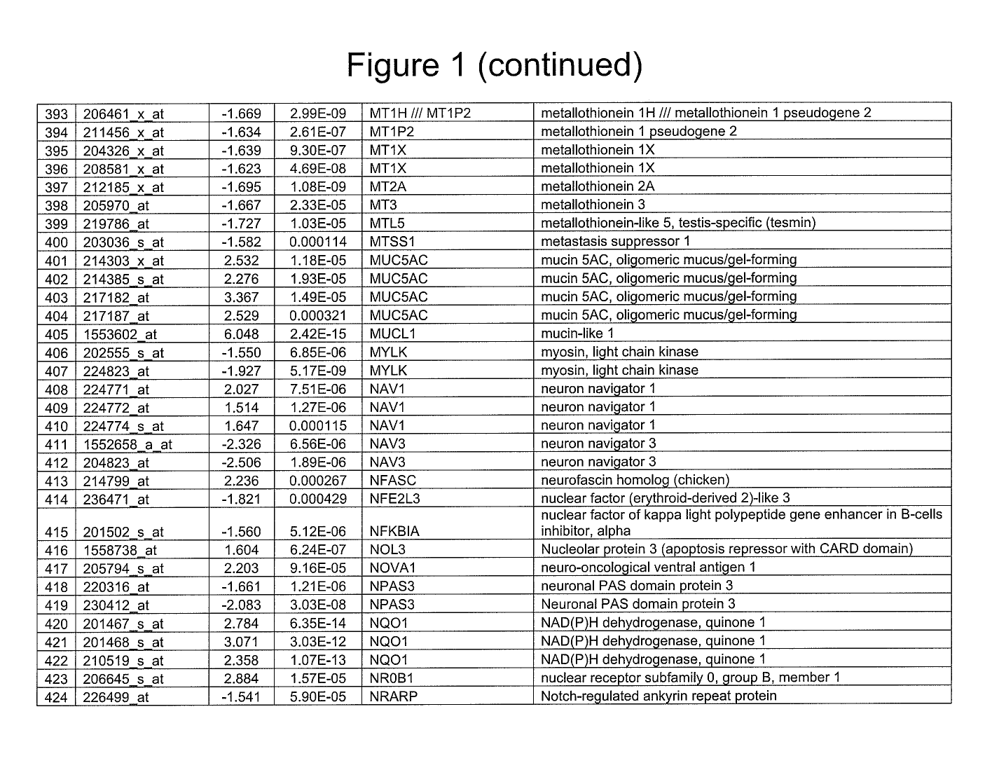

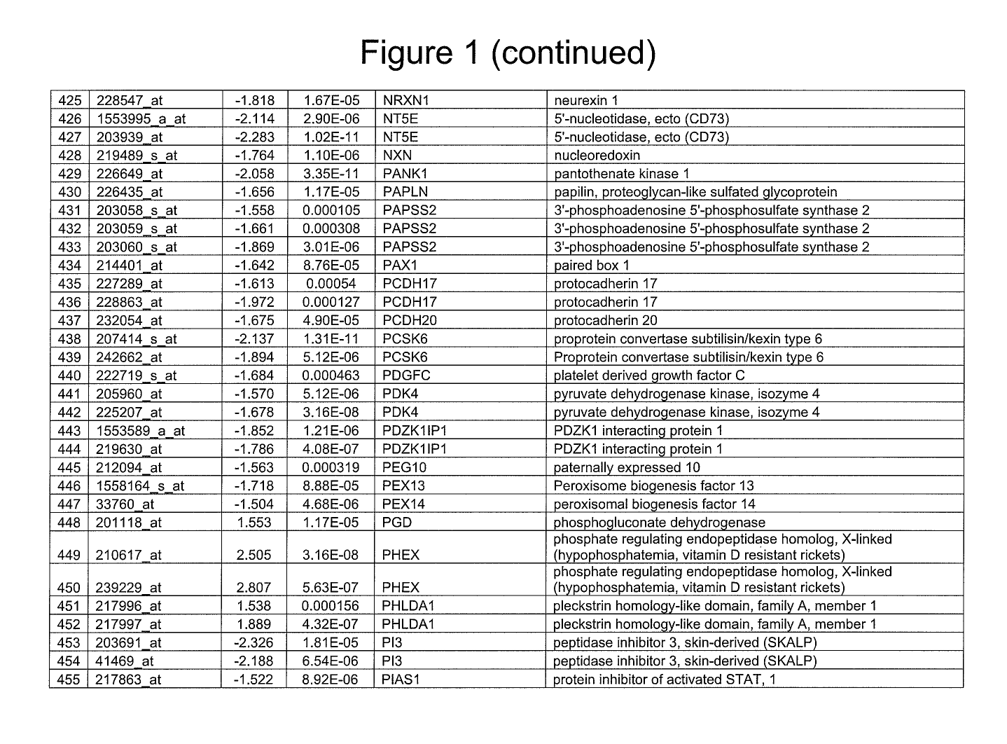

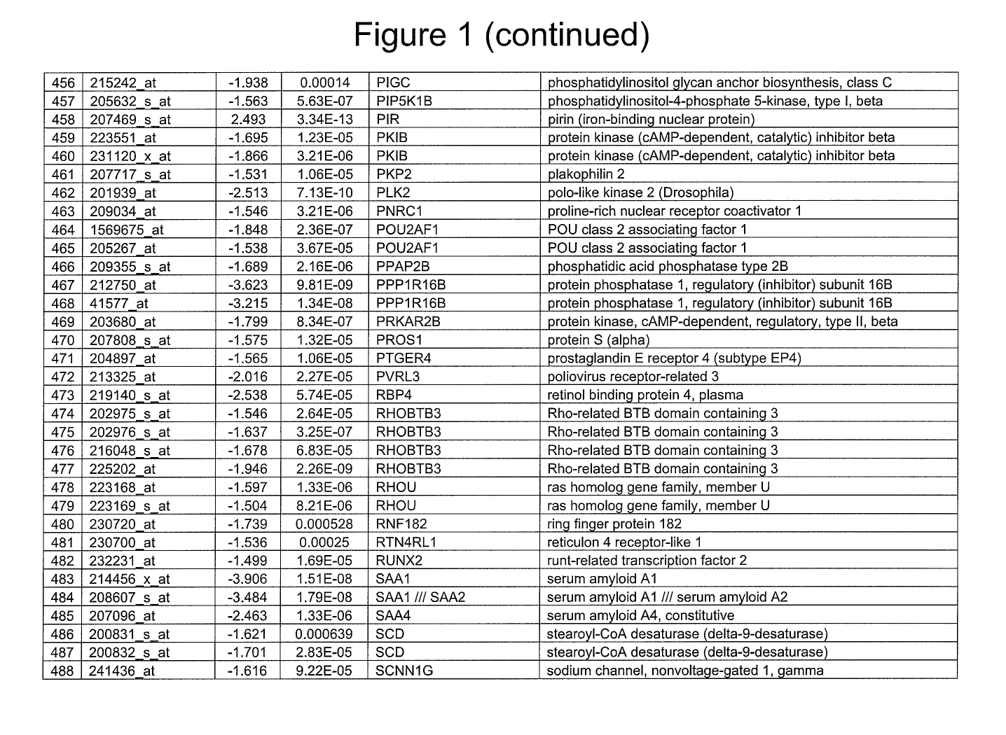

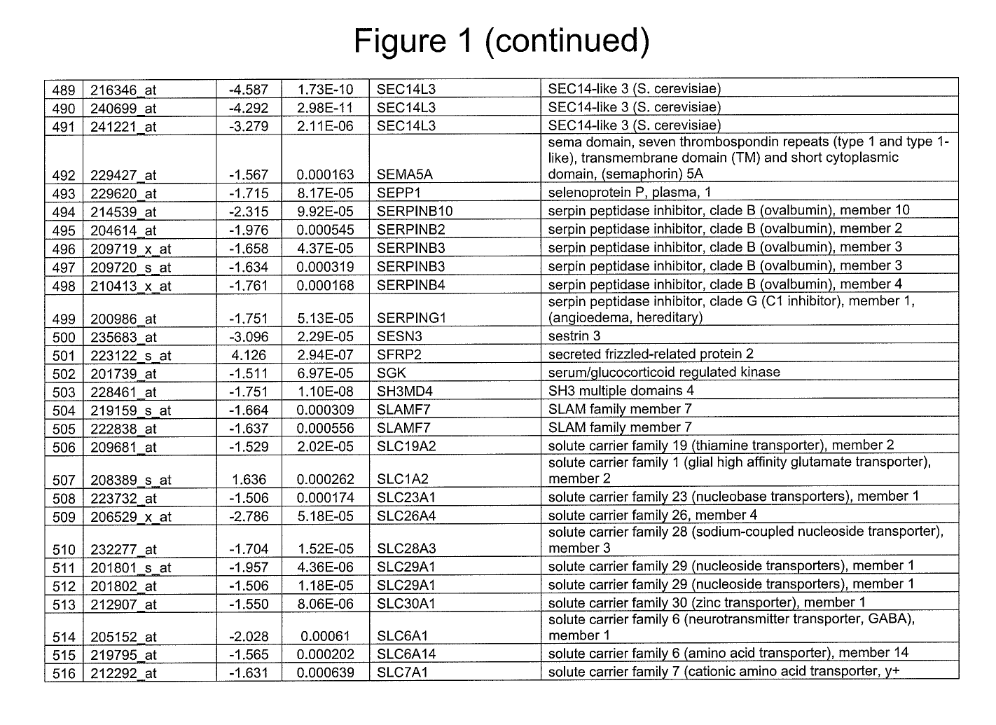

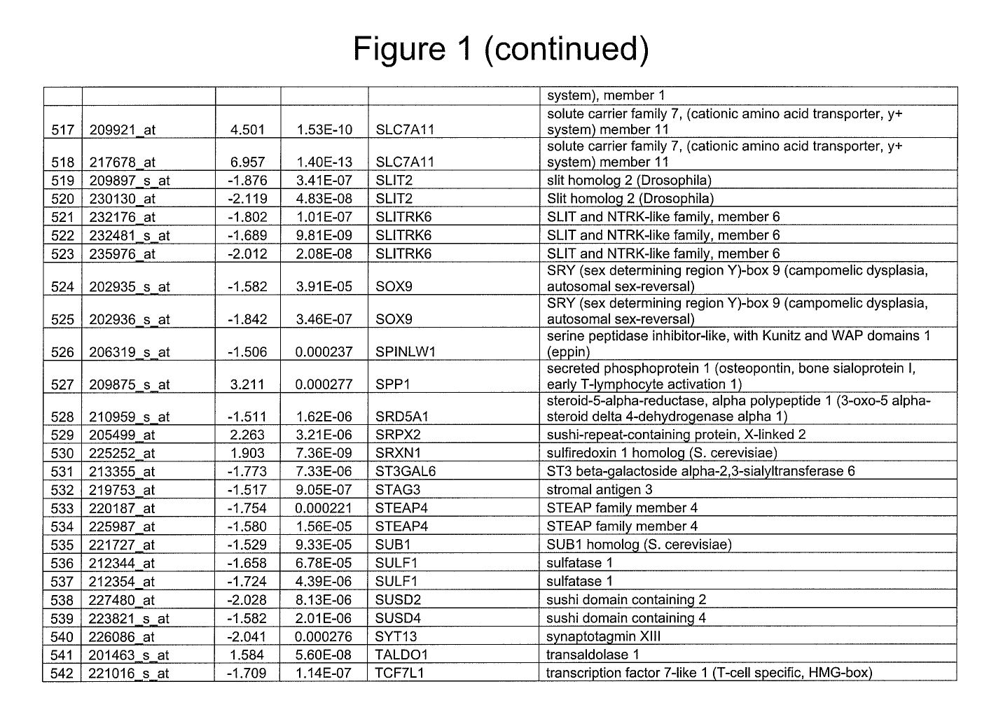

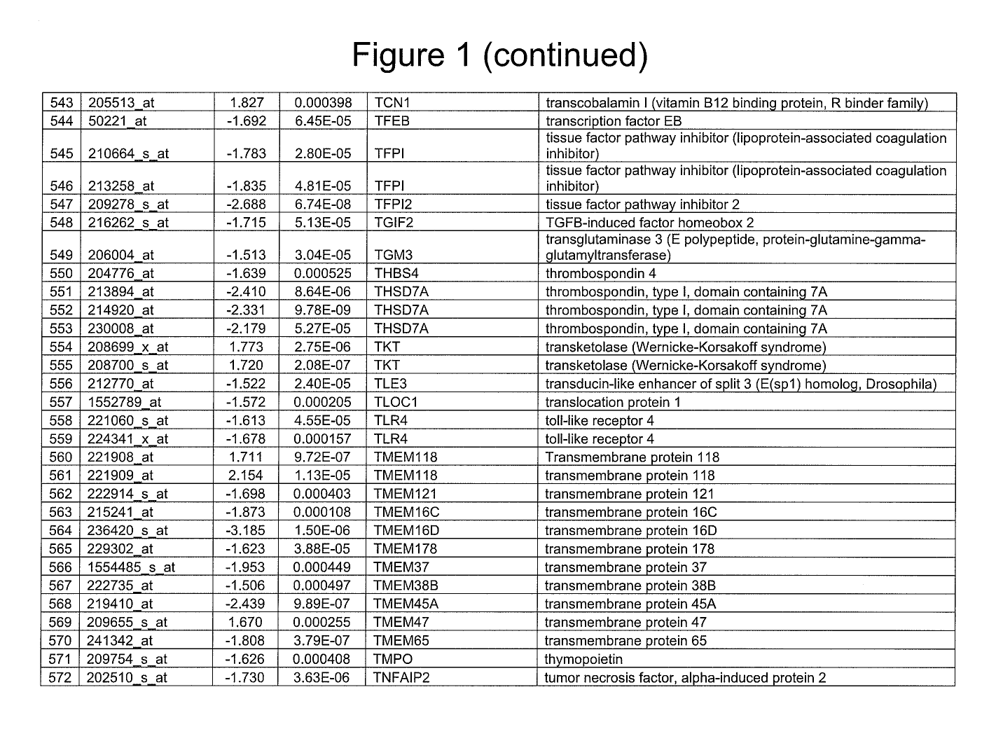

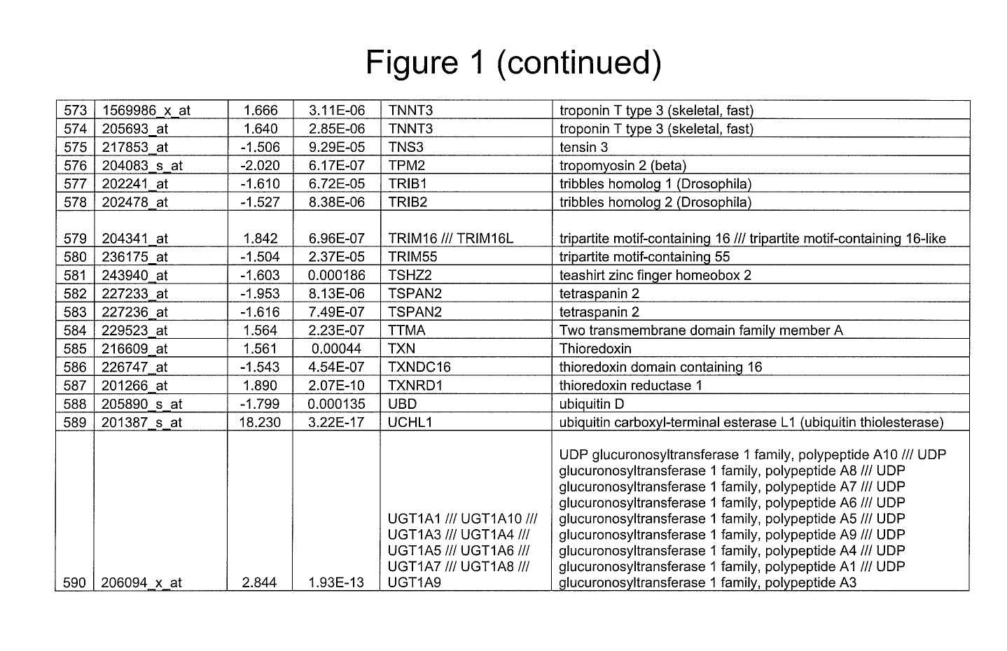

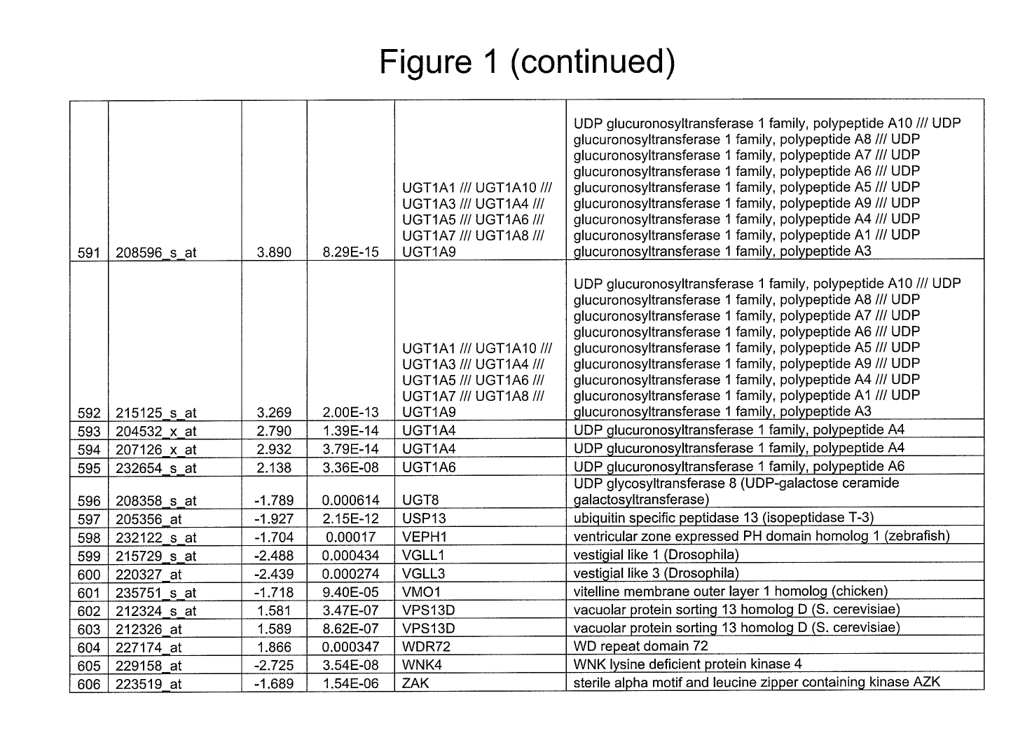

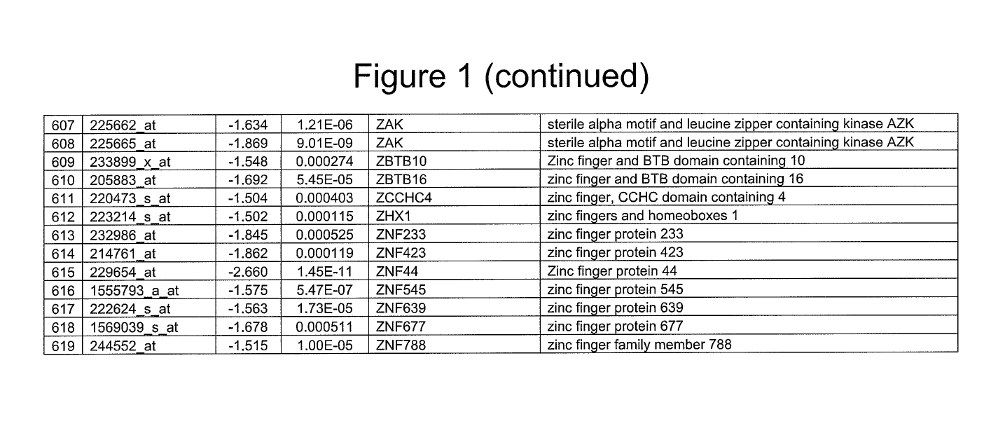

The expression pattern of one or more biomarkers associated with COPD can utilize any suitable biomarkers associated with COPD. Biomarkers associated with COPD are set forth in FIG. 1, by reference to both specific probes and corresponding genes. The expression pattern desirably utilizes one or more biomarkers of FIG. 1, exclusively or in combination with one or more other biomarkers associated with COPD. In one embodiment, the expression pattern utilizes, exclusively or in combination with one or more other biomarkers associated with COPD, one or more biomarkers selected from the group consisting of CCL2, MSRI, CD36, CSF1, LCN2, MMP2, A2M, PDG, RXRB, LAMA2, HSPA2, SSP1, CCR5, FCN1, MHC2TA, IFITM3, HTN1, MX2, IFITM3, C1R, ITGAE, COL6A2, ALCAM, VCL, ICAM3, P2RX7, RAP2A, PDE3B, RPS6KA1, MAPKAP1, RRAD, KIT, PHF16, PTPN3, ADAM10, IDE, SERPINB5, LIPA, LAMP1, FUCA1, TMF1, PBX3, HES1, SNAPC1, ZNF135, IDH1, CDA, PHGD, RNASEL, SNTB1, FABP3, SULT1C1, VAT1, CLCN7, UBE2B, DMD, KRT17, KRT7, PLTP, ASS, KIAA0368, SPAG1, MEIS4, TNNT1, HUMRIRT, NET1, BAMBI, CXADR, HUMGT198A, SORL1, SAH, SLC15A1, EML1, ERBL1, WDRL0, TNF-α, IFN-gamma, MMP-1, -9, -12, CFMCP-1, MIP-1α, CCL3, IL-8, IFN receptor 2, IL-16, MUC1, MUC15, Ser/Thr kinase 17b, Bombesin, IL-4 receptor, spondin2, TIP30, homeodomain protein kinase, CHGA, Pirin, AZGP-1, Mucin 5AC, MERTK, D111, Hes1, Hes2, Hes5, HeyL, D111, Jag1, ABP1, ARG2, C20orf96, C21orf128, C6orf118, CACNB2, CALCA, CCL17, CCL20, CHAC1, CLCA4, COL3A1, CRADD, CYS1, DNAH7, DSCAM, FCGBP, FGFR1OP2, FKBPIA, FLJ33297, FLJ36748, FLJ43663, GBP4, GPC1, GRM1, HOXA1, HS3ST3A1, HSA9761, HTR2B, IFNA4, JUNB, KCNJ1, KIAA0565, KIAA0960, KIAA1904, LDB1, LOC130355, LOC284825, LOC388335, LOC401034, LOC641941, LOC647248, LPAAT-THETA, LRRC43, MALAT1, MARCKSL1, MGC45491, MIPOL1, MT1M, MUC5B, MYL9, NCKAP1, NEB, NPTX2, PAPPA, PCDHB5, PDCD6, PER1, PLEKHA5, PRDM11, PRR12, PRR4, RAPGEFL1, RNPS1, RP11-444E17.2, RRAD, RSNL2, SBEM, SDCBP2, SERPINA3, SERPINH1, SLC13A2, SLC2A4RG, SLC39A8, SLC6A20, STK17B, TACR1, TBX1, TMSB4Y, TP73L, TPRXL, TTLL11, USH1C, USP2, VEGFB, WNT5B, ZFHX1B, ZFP36, ZNF42, ADAM-12, ARTS-1, AAA, CASP8, CTSS, LRAP, selenoprotein T, SERPINA6, SERPINB11, SFTPB, SLC34A2, TMEM1, TMEM37, TPP2, UBE1L2, UBE2, USP7, USP36, IF127, C1S, GSTA3, GSTA4, MMP1, MMP10, MMP14, OXR1, mediator of DNA damage checkpoint1 gene, BCL2-interacting protein, BCL2-associated X protein, BCL2, APAF1 interacting protein, p18, cyclin D1, transducer of ERBB2, RAP1 interacting factor homolog genes, β2 microglobulin, interleukin 6 signal transducer, aldo-keto reductase 1A1, glutamate-cysteine ligase catalytic subunit, glutamate-cysteine ligase regulatory subunit, glutamate-cysteine ligase modifier subunit, chemokine ligand 2, meprin A, tenascin C, bone morphogenetic protein 4, interferon alpha-inducible proteins 27, 6, and “44-like”, glutathione peroxidase 3, NADP+mitochondrial isocitrate dehydrogenase 2, glutathione S-transferase A2, aldo-keto reductase 1C3, aldo-keto reductase 1B1, fructose-bisphosphate aldolase A, cell division cycle 10 (CDC10), and cell division cycle 20 homolog B (CDC20B). In a preferred embodiment, the expression pattern is of one or more biomarkers selected from ABP1, ADH7, AJAP1, AKR1B10, AKR1C1, AKR1C2, AKR1C3, ALDH3A1, ANGPT1, ANPEP, AOC3, ARG2, ATP12A, ATP6VOA4, ATP6V1B1, AVPR1A, AZU1, B3GNT6, C10orf39, C10orf81, C14orf132, C20orf96, C21orf128, LOC653879, C6 orf118, CABYR, CABYR, CACNB2, CALCA, CBR1, CBR3, CCL17, CCL20, CEACAM5, CFB, CFD, CHAC1, CHEK1, ChGn, CHI3L1, CLCA4, CLDN10, CNGB1, CNN3, COL3A1, CRADD, CX3CL1, CX3CL1, CXCL2, CXCL3, CYP1A1, CYP1B1, CYP4F11, CYP4F3, CYP4X1, CYS1, D2HGDH, DEPDC6, DNAH7, DRD1, DSCAM, DTNA, DUSP1, DUSP5, EGF, ELMOD1, EPB41L2, EPHB1, FAM107A, FAM38A, FBN1, FCGBP, FGFR1OP2, FGFR2, FKBP1A, FLJ33297, FLJ36748, FLJ39051, FLJ43663, FOXA2, G6PD, GAD1, GBP4, GEM, GLRB, GPC1, GPX2, GRM1, H19, HES6, HGD, HNMT, HOXA1, HS3ST3A1, HSA9761, HSD17B2, HTR2B, IFNA4, IL27RA, IRS2, ITLN1, ITM2A, JUNB, KCNJ1, KIAA0565, KIAA0960, KIAA1904, LAMB3, LDB1, LMO4, LOC130355, LOC283177, LOC283514, LOC284825, LOC388335, LOC401034, LOC440338, LOC641941, LOC647248, LPAAT-THETA, LRRC43, LTF, MALAT1, MAOB, MARCKSL1, ME1, MEF2C, MGC45491, MIPOL1, MSRB3, MT1F, MT1G, MT1H, MT1M, MUC5AC, MUC5B, MYL9, NAV3, NCKAP1, NEB, NOVA1, NOVA1, NPTX2, NQO1, NT5E, NT5E, PAPPA, PCDHB5, PCSK6, PDCD6, PEG10, PER1, PHEX, PHLDA1, PI3, PIR, PLEKHA5, PLK2, PPAP2B, PPP1R16B, PRDM11, PRR12, PRR4, RAPGEFL1, RHOBTB3, RNPS1, RP11-444E17.2, RRAD, RSNL2, SAA1, SAA4, SBEM, SCNN1G, SDCBP2, SEC14L3, SEMA5A, SERPINA3, SERPINB10, SERPINB3, SERPINB4, SERPING1, SERPINH1, SFRP2, SFRP2, SLAMF7, SLC13A2, SLC26A4, SLC29A1, SLC2A4RG, SLC39A8, SLC6A20, SLC7A11, SLIT2, SLITRK6, SPP1, SRPX2, SRXN1, STK17B, SULF1, SUSD2, TACR1, TBX1, TFEB, TFPI, TFPI2, TMEM118, TMEM121, TMEM16D, TMEM37, TMEM45A, TMSB4Y, TP73L, TPM2, TPRXL, TTLL11, TXN, UCHL1, UGT1A10, UGT1A4, UGT1A6, USH1C, USP2, VEGFB, VEPH1, VGLL1, WDR72, WNK4, WNT5B, ZBTB16, ZFHX1B, ZFP36, ZNF42, ZNF423, and ZNF44. In yet another embodiment, the expression pattern utilizes, exclusively or in combination with one or more other biomarkers associated with COPD, one or more biomarkers selected from the group consisting of ADH7, AKR1B10, AKR1C1, AKR1C2, AKR1C3 ALDH3A1, FOXA2, G6PD, GAD1, H19, HES6, HGD, IFNA4, Intelectin1, LTF, MUC5AC, NQO1, RRAD, RSNL2, SPP1, STK17B, and UCHL1.

The standard expression pattern, to which the expression pattern associated with the sample is compared, reflects the expression pattern of biomarkers, preferably the same biomarkers utilized with the sample, in a subject (smoker and/or nonsmoker) who does not have COPD and who desirably will not acquire COPD. The standard expression pattern can be a compilation of such expression patterns from one or more such subjects.

The biomarkers can be DNA, RNA, mRNA, tRNA, and/or the proteins resulting therefrom, which are associated with COPD.

Methods of determining the expression of biomarkers are well known in the art. Suitable techniques for determining the presence and level of expression of the biomarkers in cells are within the skill in the art. According to one such method, total cellular RNA can be purified from cells by homogenization in the presence of nucleic acid extraction buffer, followed by centrifugation. Nucleic acids are precipitated, and DNA is removed by treatment with DNase and precipitation. The RNA molecules are then separated by gel electrophoresis on agarose gels according to standard techniques, and transferred to nitrocellulose filters by, e.g., the so-called “Northern” blotting technique. The RNA is then immobilized on the filters by heating. Detection and quantification of specific RNA is accomplished using appropriately labeled DNA or RNA probes complementary to the RNA in question. See, for example, Molecular Cloning: A Laboratory Manual, J. Sambrook et al., eds., 2nd edition, Cold Spring Harbor Laboratory Press, 1989, Chapter 7, the entire disclosure of which is incorporated by reference.

Methods for preparation of labeled DNA and RNA probes, and the conditions for hybridization thereof to target nucleotide sequences, are described in Molecular Cloning: A Laboratory Manual, J. Sambrook et al., eds., 2nd edition, Cold Spring Harbor Laboratory Press, 1989, Chapters 10 and 11, the disclosures of which are herein incorporated by reference. For example, the nucleic acid probe can be labeled with, e.g., a radionuclide such as ³H, ³²P, ³³P, ¹⁴C, or ³⁵S; a heavy metal; or a ligand capable of functioning as a specific binding pair member for a labeled ligand (e.g., biotin, avidin, or an antibody), a fluorescent molecule, a chemiluminescent molecule, an enzyme, or the like.

Probes can be labeled to high specific activity by either the nick translation method of Rigby et al, J. Mol. Biol., 113: 237-251 (1977), or by the random priming method of Fienberg, Anal. Biochem., 132: 6-13 (1983), the entire disclosures of which are herein incorporated by reference. The latter can be a method for synthesizing ³²P-labeled probes of high specific activity from RNA templates. For example, by replacing preexisting nucleotides with highly radioactive nucleotides according to the nick translation method, it is possible to prepare ³²P-labeled nucleic acid probes with a specific activity well in excess of 10⁸ cpm/microgram. Autoradiographic detection of hybridization can then be performed by exposing hybridized filters to photographic film. Densitometric scanning of the photographic films exposed by the hybridized filters provides an accurate measurement of biomarker levels. Using another approach, biomarker levels can be quantified by computerized imaging systems, such the Molecular Dynamics 400-B 2D Phosphorimager (Amersham Biosciences, Piscataway, N.J.).

Where radionuclide labeling of DNA or RNA probes is not practical, the random-primer method can be used to incorporate an analogue, for example, the dTTP analogue 5-(N—(N-biotinyl-epsilon-aminocaproyl)-3-aminoallyl)deoxyuridine triphosphate, into the probe molecule. The biotinylated probe oligonucleotide can be detected by reaction with biotin-binding proteins, such as avidin, streptavidin, and antibodies (e.g., anti-biotin antibodies) coupled to fluorescent dyes or enzymes that produce color reactions.

In addition to Northern and other RNA blotting hybridization techniques, determining the levels of RNA transcript can be accomplished using the technique of in situ hybridization. This technique requires fewer cells than the Northern blotting technique, and involves depositing whole cells onto a microscope cover slip and probing the nucleic acid content of the cell with a solution containing radioactive or otherwise labeled nucleic acid (e.g., cDNA or RNA) probes. This technique is particularly well-suited for analyzing tissue biopsy samples from subjects. The practice of the in situ hybridization technique is described in more detail in U.S. Pat. No. 5,427,916, the entire disclosure of which is incorporated herein by reference.

The relative number of RNA transcripts in cells also can be determined by reverse transcription of RNA transcripts, followed by amplification of the reverse-transcribed transcripts by polymerase chain reaction (RT-PCR). The levels of RNA transcripts can be quantified in comparison with an internal standard, for example, the level of mRNA from a standard gene present in the same sample. A suitable gene for use as an internal standard includes, e.g., myosin or glyceraldehyde-3-phosphate dehydrogenase (G3PDH). The methods for quantitative RT-PCR and variations thereof are within the skill in the art.

In some instances, it may be desirable to simultaneously determine the expression level of a plurality of different of biomarker genes in a sample. In certain instances, it may be desirable to determine the expression level of the transcripts of all known biomarker genes correlated with COPD. Assessing the expression patterns for numerous COPD associated biomarker genes is time consuming and requires a large amount of total RNA (at least 20 μg for each Northern blot) and autoradiographic techniques that require radioactive isotopes. To overcome these limitations, an oligolibrary in microchip format can be constructed containing a set of probe oligonucleotides specific for a set of biomarker genes, i.e., a plurality of biomarkers. In one embodiment, the oligolibrary contains probes corresponding to all known biomarkers from the human genome. The microchip oligolibrary can be expanded to include additional RNAs as they are discovered to be biomarkers associated with COPD.

The microchip can be fabricated by techniques known in the art. For example, probe oligonucleotides of an appropriate length, e.g., 40 nucleotides, are 5′-amine modified at position C6 and printed using commercially available microarray systems, e.g., the GENEMACHINE OmniGrid 100 Microarrayer and Amersham CODELINK activated slides. Labeled cDNA oligomer corresponding to the target RNAs is prepared by reverse transcribing the target RNA with labeled primer. Following first strand synthesis, the RNA/DNA hybrids are denatured to degrade the RNA templates. The labeled target cDNAs thus prepared are then hybridized to the microarray chip under hybridizing conditions, e.g., 6 times SSPE/30% formamide at 25 degrees C. for 18 hours, followed by washing in 0.75 times TNT at 37 degrees C., for 40 minutes. At positions on the array where the immobilized probe DNA recognizes a complementary target cDNA in the sample, hybridization occurs. The labeled target cDNA marks the exact position on the array where binding occurs, allowing automatic detection and quantification. The output consists of a list of hybridization events, indicating the relative abundance of specific cDNA sequences, and therefore the relative abundance of the corresponding complementary biomarker, in the subject sample. According to one embodiment, the labeled cDNA oligomer is a biotin-labeled cDNA, prepared from a biotin-labeled primer. The microarray is then processed by direct detection of the biotin-containing transcripts using, e.g., Streptavidin-Alexa647 conjugate, and scanned utilizing conventional scanning methods. Images intensities of each spot on the array are proportional to the abundance of the corresponding biomarker in the subject sample.

The use of an array has one or more advantages for mRNA expression detection. First, the global expression of several hundred genes can be identified in a same sample at one time point. Second, through careful design of the oligonucleotide probes, expression of both mature and precursor molecules can be identified. Third, in comparison with Northern blot analysis, the chip requires a small amount of RNA, and provides reproducible results using 2.5 μg of total RNA.

Protein in a sample can be detected using a variety of methods, such as protein immunostaining, immunoprecipitation, protein microarray, and Western blot, all of which are well known in the art. Immunostaining is a general term in biochemistry that applies to any use of an antibody-based method to detect a specific protein in a sample. Similarly, immunoprecipitation is the technique of precipitating an antigen out of solution using an antibody specific to that antigen. This process can be used to enrich a given protein to some degree of purity. A Western blot is a method by which protein may be detected in a given sample of tissue homogenate or extract. It uses gel electrophoresis to separate denatured proteins by mass. The proteins are then transferred out of the gel and onto a membrane (typically nitrocellulose), where they are “probed” using antibodies specific to the protein. As a result, researchers can examine the amount of protein in a given sample and compare levels between several groups. Detected protein can be quantified utilizing a Bradford Assay, which is a colorimetric protein assay, based on an absorbance shift in the dye Coomassie when bound to arginine and hydrophobic amino acid residues present in protein.

The expression pattern of biomarkers associated with COPD determined from a sample of a subject can be compared to a standard expression pattern in any suitable manner. The up-regulation of some biomarkers associated with COPD is indicative of an increased susceptibility of developing COPD, while the down-regulation of some biomarkers associated with COPD is indicative of an increased susceptibility of developing COPD. Some variation in the expression of biomarkers will be present for subjects with an increased susceptibility of developing COPD, as well as for subjects with no increased susceptibility of developing COPD. This expression of a biomarker associated with COPD in the non-smoker population can be characterized by an average (mean) value coupled with a standard deviation value. The expression of a biomarker associated with COPD that is different than the average expression for non-smokers can be considered to be abnormal and indicative of a susceptibility to develop COPD. The expression of a biomarker associated with COPD that is more than two standard deviations (i.e., +/−2 std. dev.) different than the average expression for non-smokers can be considered, with a reasonable degree of confidence (p≦0.05), to be abnormal and indicative of a susceptibility to develop COPD.

The likelihood that a smoker will develop COPD increases with an increase in the number of identified biomarkers associated with COPD that are expressed at a level that is different, e.g., desirably more than two standard deviations different, than the average expression for the biomarker for non-smokers. While, as indicated earlier, one or more such biomarkers can be examined for purposes of the invention in connection with a sample obtained from a subject, an evaluation of an increased number of biomarkers associated with COPD (e.g., 50 or more, 100 or more, 200 or more, 300 or more, 400 or more, or 500 or more) provides an increased degree of accuracy concerning the evaluation of whether or not the subject will develop COPD, with an increasing percentage (e.g., 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 92% or more, 95% or more, 96% or more, 97% or more, 98% or more, or 100%) of such biomarkers having an expression more than two standard deviations different than the average expression for the biomarkers for non-smokers, providing an increased likelihood of the subject's developing COPD. For example, if 30% or more (e.g., 35% or more, 40% or more, or even 45% or more) of the 384 genes (evaluated by way of 619 probes) identified in FIG. 1 are expressed in a subject at a level more than 2 standard deviations different from the normal expression of those genes, then there is a significant likelihood that the subject will develop COPD.

The use of an index value to characterize the expression pattern determined from a sample obtained from a subject can prove useful with respect to evaluating the susceptibility of the subject to develop COPD. Such an index value can have any suitable form and can simply involve according a value of 1 to any gene with an expression that is more than two standard deviations different from the average expression of that gene. For multiple probes associated with a single gene, the expression determined by each probe for the same gene can be accorded a proportional value adding up to 1 for the gene (e.g., an expression that is more than two standard deviations different than the normal average expression for three of four probes associated with a single gene would be accorded a value of 0.75, which is based on 3 probe-based instances of abnormal expressions×1 gene/4 probes). Alternatively, a value accorded to a gene where the expression is more than two standard deviations different than the normal average expression can be the number reflecting the fold difference between the determined expression and the normal average expression (e.g., an expression level for a biomarker that is three standard deviations different from the normal expression level would be accorded a value of 1.75). The index value can clearly discriminate among smokers and non-smokers. In addition, healthy smokers can be subcategorized based on the index value as having a high response to the stress of smoking, an intermediate response to the stress of smoking, or a low response which is more similar to that of non-smokers.

The invention further provides a method of treating the smoker who is determined to be likely to develop COPD. The method comprises administering an effective amount of a substance to the smoker to either (a) down-regulate one or more biomarkers (as discussed herein) whose up-regulation led to the determination that the smoker likely would develop COPD, or (b) up-regulate one or more biomarkers whose down-regulation led to the determination that the smoker likely would develop COPD. The substance can be any suitable substance that is known in the art to treat smoking-related diseases such as COPD. Examples of such substances include pharmaceutical agents such as antiinflammatories, bronchodilators (e.g., β2 agonists, M₃ muscarinic antagonists, cromones, leukotriene antagonists, and xanthines), corticosteroids (e.g., beclomethasone, mometasone, and fluticasone), monoclonal antibodies (e.g., infliximab, adalimumab), vitamins, antibiotics, mucolytics, and TNF antagonists (e.g., etanercept). The substance also can be a gene therapy composition that specifically targets the mRNAs described herein. The method of treating the smoker can involve other traditional treatments of COPD, including, for example, stem cells, vaccination against influenza, smoking cessation, surgery (e.g., lung transplant, lung volume reduction surgery), home oxygen therapy, pulmonary rehabilitation, vaccination against pneumococcus, and exercise.

As discussed above, gene therapeutic techniques can be used independently to treat the subject with COPD, or in conjunction with any of the above-mentioned treatments. In this regard, the invention provides a composition comprising a therapeutically effective amount of a nucleic acid complementary to at least one of the biomarkers associated with COPD, and a pharmaceutically acceptable carrier. The nucleic acid can be complementary to one or more biomarkers whose up-regulation led to a determination that the smoker likely would develop COPD. In this case, the composition binds and renders ineffective (i.e., inhibits) the biomarkers. Alternatively, the nucleic acid can be complementary to one or more biomarkers whose down-regulation led to a determination that the smoker likely would develop COPD. In each case, the composition alters the expression of the gene coding for the biomarkers, thereby altering, and preferably normalizing, the amounts or levels of biomarkers produced, the technology for which is well known within the art.

In the practice of the present treatment methods, an effective amount of at least one composition which inhibits at least one of the biomarkers also can be administered to the subject. As used herein, “inhibiting” means that one or more biomarker levels and/or the production of one or more biomarker gene products from genes associated with COPD after treatment is less than prior to treatment. In another embodiment, a composition that increases the expression of one or more of the biomarkers is administered. One skilled in the art can readily determine whether biomarker levels or gene expression has been inhibited or increased from sample to sample taken over a period of time using, for example, the techniques for determining biomarker transcript level discussed above.

As used herein, an “effective amount” of a substance that treats COPD or a composition that inhibits biomarkers or biomarker gene expression is an amount sufficient to prevent, delay the onset, or reverse symptoms of a subject with COPD. One skilled in the art can readily determine an effective amount of an inhibiting substance or composition to be administered to a given subject, by taking into account factors such as the size and weight of the subject, the extent of disease penetration, the age, health, and sex of the subject, the route of administration; and whether the administration is regional or systemic.

One skilled in the art also can readily determine an appropriate dosage regimen for administering a composition that alters biomarker levels or gene expression to a given subject. For example, the composition can be administered to the subject once (e.g. as a single injection or deposition). Alternatively, the composition can be administered, for instance, once or twice daily, monthly, bimonthly, or biannually. The administration of the treatment to a subject can be for a period ranging from days, weeks, months, or years. In certain embodiments, the treatment continues throughout the life of the subject. Where a dosage regimen comprises multiple administrations, it is understood that the effective amount of the composition administered to the subject can comprise the total amount of composition administered over the entire dosage regimen.

Suitable compositions for inhibiting biomarker gene expression include double-stranded RNA (such as short- or small-interfering RNA or “siRNA”), antisense nucleic acids, and enzymatic RNA molecules such as ribozymes. Each of these compositions can be targeted to a given biomarker gene product and destroy or induce the destruction of the target biomarker gene product.

For example, expression of a given biomarker gene can be inhibited by inducing RNA interference of the biomarker gene with an isolated double-stranded RNA (“dsRNA”) molecule which has at least 90%, for example, at least 95%, at least 98%, at least 99%, or 100%, sequence homology with at least a portion of the biomarker gene product. In a preferred embodiment, the dsRNA molecule is a “short or small interfering RNA” or “siRNA.”

siRNA useful in the present methods comprise short double-stranded RNA from about 17 nucleotides to about 29 nucleotides in length, preferably from about 19 to about 25 nucleotides in length. The siRNA comprise a sense RNA strand and a complementary antisense RNA strand annealed together by standard Watson-Crick base-pairing interactions (hereinafter “base-paired”). The sense strand comprises a nucleic acid sequence which is substantially identical to a nucleic acid sequence contained within the target biomarker gene product.

As used herein, an siRNA “substantially identical” to a target sequence contained within the target nucleic sequence is a nucleic acid sequence that is identical to the target sequence or differs from the target sequence by one or two nucleotides. The sense and antisense strands of the siRNA can comprise two complementary, single-stranded RNA molecules, or can comprise a single molecule in which two complementary portions are base-paired and are covalently linked by a single-stranded “hairpin” area.

The siRNA also can be altered RNA that differs from naturally-occurring RNA by the addition, deletion, substitution, and/or alteration of one or more nucleotides. Such alterations can include the addition of non-nucleotide material, such as to the end(s) of the siRNA or to one or more internal nucleotides of the siRNA, or modifications that make the siRNA resistant to nuclease digestion, or the substitution of one or more nucleotides in the siRNA with deoxyribonucleotides.

One or both strands of the siRNA also can comprise a 3′ overhang. As used herein, a “3′ overhang” refers to at least one unpaired nucleotide extending from the 3′-end of a duplexed RNA strand. Thus, in one embodiment, the siRNA comprises at least one 3′ overhang of from 1 to about 6 nucleotides (which includes ribonucleotides or deoxyribonucleotides) in length, preferably from 1 to about 5 nucleotides in length, more preferably from 1 to about 4 nucleotides in length, and particularly preferably from about 2 to about 4 nucleotides in length. In a preferred embodiment, the 3′ overhang is present on both strands of the siRNA, and is 2 nucleotides in length. For example, each strand of the siRNA can comprise 3′ overhangs of dithymidylic acid (“TT”) or diuridylic acid (“uu”).

The siRNA can be produced chemically or biologically, or can be expressed from a recombinant plasmid or viral vector, as described above for the isolated biomarker gene products. Exemplary methods for producing and testing dsRNA or siRNA molecules are described in U.S. Patent Application Publication No. 2002/0173478 and U.S. Pat. No. 7,148,342, the entire disclosures of which are herein incorporated by reference.

Expression of a given biomarker gene also can be inhibited by an antisense nucleic acid. As used herein, an “antisense nucleic acid” refers to a nucleic acid molecule that binds to target RNA by means of RNA-RNA or RNA-DNA or RNA-peptide nucleic acid interactions, which alters the activity of the target RNA. Antisense nucleic acids suitable for use in the present methods are single-stranded nucleic acids (e.g., RNA, DNA, RNA-DNA chimeras, PNA) that generally comprise a nucleic acid sequence complementary to a contiguous nucleic acid sequence in a biomarker gene product. Preferably, the antisense nucleic acid comprises a nucleic acid sequence that is 50-100% complementary, more preferably 75-100% complementary, and most preferably 95-100% complementary, to a contiguous nucleic acid sequence in a biomarker gene product.

Antisense nucleic acids can also contain modifications to the nucleic acid backbone or to the sugar and base moieties (or their equivalent) to enhance target specificity, nuclease resistance, delivery, or other properties related to efficacy of the molecule. Such modifications include cholesterol moieties, duplex intercalators such as acridine, or the inclusion of one or more nuclease-resistant groups.

Antisense nucleic acids can be produced chemically or biologically, or can be expressed from a recombinant plasmid or viral vector, as described above for the isolated biomarker gene products. Exemplary methods for producing and testing are within the skill in the art, as disclosed in, for example, Stein, Science, 261:1004 (1993), and U.S. Pat. No. 5,849,902, the entire disclosures of which are herein incorporated by reference.

Expression of a given biomarker gene also can be inhibited by an enzymatic nucleic acid. As used herein, an “enzymatic nucleic acid” refers to a nucleic acid comprising a substrate binding region that has complementarity to a contiguous nucleic acid sequence of a biomarker gene product, and which is able to specifically cleave the biomarker gene product. Preferably, the enzymatic nucleic acid substrate binding region is 50-100% complementary, more preferably 75-100% complementary, and most preferably 95-100% complementary to a contiguous nucleic acid sequence in a biomarker gene product. The enzymatic nucleic acids can also comprise modifications at the base, sugar, and/or phosphate groups. An exemplary enzymatic nucleic acid for use in the present methods is a ribozyme.

The enzymatic nucleic acids can be produced chemically or biologically, or can be expressed from a recombinant plasmid or viral vector, as described above for the isolated biomarker gene products. Exemplary methods for producing and testing dsRNA or siRNA molecules are described in Werner, Nucl. Acids Res., 23: 2092-96 (1995); Hammann, Antisense and Nucleic Acid Drug Dev., 9: 25-31 (1999); and U.S. Pat. No. 4,987,071, the entire disclosures of which are herein incorporated by reference.

The administration of at least one substance that treats COPD or a composition for inhibiting at least one biomarker or expression of a biomarker gene will prevent, delay the onset, or reverse the symptoms of COPD. By preventing COPD it is meant that a smoker identified as likely to develop COPD is treated and does not develop COPD. By delaying the onset of COPD it is meant that a smoker who is identified as likely to develop COPD does develop COPD but does so later than would otherwise have occurred. By reversing the symptoms of COPD, it is meant that a smoker who exhibits symptoms of COPD experiences full or partial relief of those symptoms following treatment.

The inventive substances or compositions can be administered to a subject by any means suitable for delivering these compositions to lungs of the subject. For example, the substances or compositions can be administered by methods suitable to transfect cells of the subject with these substances or compositions. Preferably, the cells are transfected with a plasmid or viral vector comprising sequences encoding at least one biomarker gene product or biomarker gene expression inhibiting composition.

Transfection methods for eukaryotic cells are well known in the art, and include, e.g., direct injection of the nucleic acid into the nucleus or pronucleus of a cell, electroporation, liposome transfer or transfer mediated by lipophilic materials, receptor-mediated nucleic acid delivery, bioballistic or particle acceleration, calcium phosphate precipitation, and transfection mediated by viral vectors.

For example, cells can be transfected with a liposomal transfer composition, e.g., DOTAP (N-[1-(2,3-dioleoyloxy)propyl]-N,N,N-trimethyl-ammonium methylsulfate, Boehringer-Mannheim) or an equivalent, such as LIPOFECTIN. The amount of nucleic acid used is not critical to the practice of the invention; acceptable results may be achieved with 0.1-100 micrograms of nucleic acid/10⁵ cells. For example, a ratio of about 0.5 micrograms of plasmid vector in 3 micrograms of DOTAP per 10⁵ cells can be used.

The substance or composition also can be administered to a subject by any suitable enteral or parenteral administration route. Suitable enteral administration routes for the present methods include, e.g., oral or intranasal delivery. Suitable parenteral administration routes include, e.g., intravascular administration (e.g., intravenous bolus injection, intravenous infusion, intra-arterial bolus injection, intra-arterial infusion, and catheter instillation into the vasculature); subcutaneous injection or deposition, including subcutaneous infusion (such as by osmotic pumps); direct application to the tissue of interest (i.e., lung tissue), for example by a catheter or other placement device (e.g., an implant comprising a porous, non-porous, or gelatinous material); and inhalation.

In the present methods, the composition can be administered to the subject either as naked RNA, in combination with a delivery reagent, or as a nucleic acid (e.g., a recombinant plasmid or viral vector) comprising sequences that express the biomarker gene product or expression inhibiting composition. Suitable delivery reagents include, e.g., the Mirus Transit TKO lipophilic reagent, lipofectin, lipofectamine, cellfectin, polycations (e.g., polylysine), and liposomes.

Recombinant plasmids and viral vectors comprising sequences that express the biomarker or biomarker gene expression inhibiting compositions, and techniques for delivering such plasmids and vectors to a lung, are discussed above.

In a preferred embodiment, liposomes are used to deliver a biomarker or biomarker gene expression-inhibiting composition (or nucleic acids comprising sequences encoding them) to a subject. Liposomes can also increase the blood half-life of the gene products or nucleic acids.

Liposomes suitable for use in the invention can be formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example, as described in Szoka, Ann. Rev. Biophys. Bioeng., 9: 467 (1980); and U.S. Pat. Nos. 4,235,871, 4,501,728, 4,837,028, and 5,019,369, the entire disclosures of which are herein incorporated by reference.

The liposomes for use in the present methods can comprise a ligand molecule that targets the liposome to lungs (i.e., small airways and/or large airways). Ligands which bind to receptors prevalent in the lungs, such as monoclonal antibodies that bind small airway epithelial cells, are preferred.

The substances or compositions of the invention may include a pharmaceutically acceptable carrier. The term “pharmaceutically acceptable carrier” as used herein means one or more compatible solid or liquid fillers, diluents, other excipients, or encapsulating substances which are suitable for administration into a human or veterinary patient. The term “carrier” denotes an organic or inorganic ingredient, natural or synthetic, with which the active ingredient is combined to facilitate the application. The components of the pharmaceutical compositions also are capable of being co-mingled with one or more of active components, and with each other, in a manner so as not to substantially impair the desired pharmaceutical efficacy. “Pharmaceutically acceptable” materials are capable of administration to a patient without the production of undesirable physiological effects such as nausea, dizziness, rash, or gastric upset. It is, for example, desirable for a therapeutic composition comprising pharmaceutically acceptable excipients not to be immunogenic when administered to a human patient for therapeutic purposes.

The pharmaceutical compositions may contain suitable buffering agents, including, for example, acetic acid in a salt, citric acid in a salt, boric acid in a salt, and phosphoric acid in a salt. The pharmaceutical compositions also optionally can contain suitable preservatives, such as benzalkonium chloride, chlorobutanol, parabens, and thimerosal.

The pharmaceutical compositions conveniently can be presented in unit dosage form and can be prepared by any of the methods well known in the art of pharmacy. Such methods include the step of bringing the active agent into association with a carrier that constitutes one or more accessory ingredients. In general, the compositions are prepared by uniformly and intimately bringing the active composition into association with a liquid carrier, a finely divided solid carrier, or both, and then, if necessary, shaping the product.

Compositions suitable for parenteral administration conveniently comprise a sterile aqueous preparation of the inventive composition, which is preferably isotonic with the blood of the recipient. This aqueous preparation can be formulated according to known methods using suitable dispersing or wetting agents and suspending agents. The sterile injectable preparation also can be a sterile injectable solution or suspension in a non-toxic parenterally-acceptable diluent or solvent, for example, as a solution in 1,3-butane diol. Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid can be used in the preparation of injectables. Carrier formulation suitable for oral, subcutaneous, intravenous, intramuscular, etc. administrations can be found in Remington's Pharmaceutical Sciences, Mack Publishing Co., Easton, Pa., which is incorporated herein in its entirety by reference thereto.

The delivery systems of the invention are designed to include time-released, delayed release, or sustained release delivery systems such that the delivering of the inventive composition occurs prior to, and with sufficient time to cause, sensitization of the site to be treated. The inventive composition can be used in conjunction with other therapeutic agents or therapies. Such systems can avoid repeated administrations of the inventive composition, thereby increasing convenience to the subject and the physician, and may be particularly suitable for certain compositions of the invention.

Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are lipids including sterols such as cholesterol, cholesterol esters, and fatty acids or neutral fats such as mono-di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the active composition is contained in a form within a matrix such as those described in U.S. Pat. Nos. 4,452,775, 4,667,014, 4,748,034, and 5,239,660 and (b) diffusional systems in which an active component permeates at a controlled rate from a polymer such as described in U.S. Pat. Nos. 3,832,253 and 3,854,480. In addition, pump-based hardware delivery systems can be used, some of which are adapted for implantation.

The invention further provides a method of determining the efficacy of a treatment for COPD. The method comprises (a) providing a sample obtained from a subject that is undergoing treatment for COPD, (b) analyzing the sample to determine the expression pattern of one or more biomarkers associated with COPD, and (c) comparing the expression pattern determined from the sample with a standard expression pattern to determine whether the treatment for COPD has or has not been effective.

The standard with which the sample is compared can be a normalized standard and/or can be a sample taken at an earlier time from the same subject. In this regard, the subject's sample may be compared to a normalized population of smokers that do not suffer from COPD (i.e., phenotypically normal smokers), nonsmokers who do not suffer from COPD, as well as early stage COPD smokers, and COPD smokers (e.g., late stage COPD smokers). Alternatively, the sample may be compared to a sample taken from the same subject prior to treatment or the subject after treatment has commenced (i.e., the subject at an earlier time).

The following examples further illustrate the invention but, of course, should not be construed as in any way limiting its scope.

### Example 1

This example compares the MERTK expression in normal nonsmokers and phenotypically normal smokers.

Rationale: Mononuclear phagocytes play an important role in the removal of apoptotic cells by expressing cell surface receptors that recognize and remove apoptotic cells. One newly recognized receptor in this class is Mer tyrosine kinase (MERTK), a 110 kDa single chain transmembrane tyrosine kinase receptor, linked to apoptosis. Based on the knowledge that cigarette smoking is associated with increased airway epithelial cell turnover, it was hypothesized that alveolar macrophages (AM) of cigarette smokers exhibit enhanced expression of the MERTK gene.

Methods: AM obtained by bronchoalveolar lavage of normal nonsmokers (n=11) and phenotypic normal smokers (n=13, 36±6 pack yr) were assessed for mRNA expression of all known apoptotic cell removal receptors using Affymetrix HG-U133 Plus 2.0 chips with TAQMAN RT-PCR confirmation.

Results: Comparing nonsmokers and smokers for the expression of the known apoptotic cell-removal receptors CD14, CD36, CD44, vitronectin, complement component 3, low density lipoprotein-related receptor, and MERTK, the most striking smoking-induced changes were with MERTK, with smoker AM having 3.6-fold up-regulation in MERTK mRNA levels (smoker vs. nonsmoker, p<0.001). This observation was confirmed by TAQMAN RT-PCR analysis (smoker vs. nonsmoker 9.5-fold, p<0.02).

Conclusions: MERTK, a cell surface receptor that recognizes apoptotic cells, is expressed on AM, and its expression is up-regulated in AM of cigarette smokers. This result may reflect an increased demand for removal of apoptotic cells in smokers, which has implications for the development of COPD inasmuch as COPD is a disorder associated with dysregulated apoptosis of airway epithelium.

### Example 2

This example compares the expression of Notch3, the ligand D111, and 4 downstream genes, Hes1, Hes2, Hes5, and HeyL, in nonsmokers and healthy smokers.

Rationale: The small airways are the earliest sites of disease in smoking-induced COPD, where the differentiation status of the epithelium changes, leading to abnormal epithelial morphology. In the context that the Notch signaling pathway acts to block epithelial differentiation in lung morphogenesis, it was hypothesized that smoking-induced epithelial injury would lead to down-regulation of Notch signaling and a permissive state toward differentiation.

Methods: Small airway (10^{th}-12^{th} generation) epithelium was obtained via bronchoscopy and brushing of nonsmokers (n=13), healthy smokers (n=18, 34±19 pack-yr), and individuals with COPD GOLD stage I-III (n=11, 47±27 pack-yr). Affymetrix HG U133A Plus 2.0 microarrays were used to assess expression of 4 receptors, 5 ligands, and 6 transcriptionally controlled downstream effector genes in the Notch pathway.

Results: Compared to nonsmokers, normal smokers down-regulated the receptor Notch3, the ligand D111, and 4 downstream genes, Hes1, Hes2, Hes5, and HeyL (all fold changes >1.5, p<0.05). Individuals with COPD down-regulated Notch3 to a greater extent than did normal smokers and down-regulated Notch2 and Notch4 (p<0.01). Similarly, individuals with COPD down-regulated D111 more than normal smokers, and down-regulated the ligand Jag1 (p<0.01). There was no significant difference in expression of the downstream Notch genes among individuals with COPD and normal smokers (p>0.05).

Conclusions: The data shows that smoking leads to down-regulation of Notch receptors, Notch ligands, and downstream genes. Individuals with COPD down-regulate more receptors and ligands, and to a greater degree, than normal smokers, but down-regulate downstream genes to the same extent. The data is consistent with the hypothesis that the Notch pathway plays a role in the small airway epithelial changes linked to smoking and COPD.

### Example 3

This example compares DEFB1 and LTF expression in nonsmokers and phenotypically normal smokers.

Rationale: Among the multiple defenses of the airways to bacterial infection are secreted antibacterial peptides that are toxic to bacteria and/or function to enhance bacterial clearance. Since healthy smokers and smokers with COPD have an increased incidence of bacterial airway infection, it was hypothesized that smoking likely has significant effects on small airway epithelial expression of antibacterial peptides.

Methods: Small (10^{th}-12^{th} order) airway epithelium obtained by fiberoptic bronchoscopy of nonsmokers (n=13), phenotypic normal smokers (n=18) and smokers with COPD (GOLD I-III, n=11) was assessed for mRNA levels of all known secreted antimicrobial peptide-encoding genes using Affymetrix HG-U133 Plus 2.0 arrays. Gene expression was considered significant if expressed genes (present in ≧50% of individuals in any one group) were up- or down-regulated>2-fold (p<0.05) between nonsmokers and smokers (healthy and/or COPD).

Results: Out of a list of expressed defensins, RNAses, S100 calcium binding proteins, lysozyme, and lactotransferrin, the expression of defensin B1 (DEFB 1) and lactotransferrin (LTF) were modulated in normal smokers and COPD smokers. Interestingly, the smoking-induced changes were in the opposite direction. DEFB1 expression was increased in the small airway epithelium in normal smokers and COPD smokers (p<0.02, all comparisons) while LTF expression was decreased in the small airway epithelium in normal smokers and COPD smokers (p<0.001, all comparisons).

Conclusions: While antimicrobial peptides play an important role in protecting the airway epithelium, the stress of cigarette smoking has discordant effects on the small airway epithelial expression of at least 2 antimicrobial peptide genes, likely contributing to the validity of airway defenses induced by smoking.

### Example 4

This example compares the expression of Intelectin 1 in nonsmokers, smokers, and individuals with early and late COPD.

Rationale: Intelectin 1, a recently described 313 amino acid human lectin, participates in the innate immune response by recognizing and binding to galactofuranosyl residues in cell walls of bacteria. Although overexpression of intelectin 1 has been reported in the bronchial epithelium of individuals with asthma, based on the knowledge that cigarette smoking is associated with increased susceptibility to respiratory tract infections, it was hypothesized that cigarette smoking may suppress the gene expression levels of intelectin 1.

Methods: Affymetrix HG U133 Plus 2.0 microarrays were used to survey expression of anti-microbial peptides and evaluate intelectin 1 gene expression in the small (10^{th} to 12^{th} order bronchi) airway epithelium obtained by bronchoscopy from 39 individuals, including 12 normal nonsmokers, 12 phenotypic normal smokers, 9 individuals with early chronic obstructive pulmonary disease (COPD), and 6 individuals with established COPD. TAQMAN RT-PCR was used to confirm changes in gene expression.

Results: Compared to normal nonsmokers, intelectin 1 gene expression was decreased in small airway epithelium of normal smokers (13.3-fold decrease, p<0.01), smokers with early COPD (14.1-fold decrease, p<0.01), and smokers with established COPD (6.8-fold decrease, p<0.01). This down-regulation of expression was confirmed using TAQMAN RT-PCR (14.6-fold decrease, p<0.01).

Conclusions: Intelectin 1 is an epithelial molecule that plays a role in defense against invading pathogens through the specific recognition of components of the cell walls of bacteria. The down-regulation of expression of intelectin 1 in response to cigarette smoking may contribute to the increase in susceptibility to infections observed in smokers.

### Example 5

This example compares the expression of cytochrome P450 1B1, mucin 5AC, maternally imprinted H19, and glutamate decarboxylase 1 in healthy nonsmokers and healthy smokers.

Rationale: Despite overwhelming data that cigarette smoking causes COPD and lung cancer, only 15-25% of chronic smokers develop these disorders. It was hypothesized that there must be variability among smokers in expression levels of protective/susceptibility genes in the small airway epithelium, which is the initial site of these disorders, in response to the stress of smoking.

Methods: Affymetrix HG U133A Plus 2.0 microarrays were used to evaluate gene expression in small airway epithelium obtained by bronchoscopy of 13 healthy nonsmokers and 15 healthy smokers. Genes differentially expressed in the 2 groups were identified by fold-change and Welch t-test with Benjamini-Hochberg correction (p<0.05), and then ranked by coefficient of variation (CV). This gene list was validated with an independent dataset derived from small airways of smokers with COPD (n=16).

Results: Of 21,000 probe sets expressed in small airway epithelium, 125 (0.6%) were differentially expressed in smokers vs. nonsmokers. Among these, 46 (representing 36 unique genes) had a CV>50%, with 19 genes up- and 17 genes down-regulated in smokers. For 4 genes (cytochrome P450 1B1, mucin 5AC, maternally imprinted H19, and glutamate decarboxylase 1), expression levels varied>50-fold among smokers. Of the 46 probe-sets with CV>50% in healthy smokers, 42 (91%) also showed a coefficient of variation>50% in the independent dataset of smokers with COPD.

Conclusions: There are identifiable genes whose expression level is highly variable in two independent samples of airway epithelium from chronic smokers. This variability is likely caused by genetic differences, and these genes with variable expression levels in response to smoking represent putative candidate genes for susceptibility/protection from disease.

### Example 6

This example compares the expression of AZGP-1 in healthy nonsmokers and healthy smokers.

Rationale: Increased body fat and weight gain are observed following smoking cessation and loss of body fat stores is frequently observed in patients with COPD. Based on the knowledge that alpha 2 zinc glycoprotein-1 (AZGP-1), which is a soluble protein found in secretory epithelial cells and adipose tissue, has been associated with loss of adipose body stores, including increased AZGP-1 urine levels in individuals with cancer-induced cachexia, and loss of body fat in mice injected with AZGP-1, it was hypothesized that cigarette smoking up-regulates AZGP-1 gene expression in the bronchial epithelium.

Methods: Large airway epithelium of 8 nonsmokers and 8 phenotypically normal smokers obtained by bronchoscopy and brushing were assessed for AZGP-1 mRNA levels using Affymetrix HG-U133 Plus 2.0 microarrays with TAQMAN RT-PCR confirmation. Large airway biopsies were evaluated using immunohistochemistry to assess AZGP-1 protein expression.

Results: AZGP-1 mRNA levels were significantly up-regulated in the airway epithelium of smokers compared to nonsmokers (2.7-fold change, p<0.02). TAQMAN RT-PCR confirmed the changes observed in microarray data (2.8-fold change, p<0.02). Immunohistochemistry demonstrated expression of AZGP-1 in goblet cells as well as in neuroendocrine cells.

Conclusions: The molecular mechanisms underlying the relationship between weight loss and smoking, and the weight loss associated with COPD are not well understood. The up-regulation of AZGP-1 gene expression in the large airway epithelium of smokers may represent a mechanism underlying the associated weight loss linked to smoking.

### Example 7

This example compares the expression of UCHL1 in nonsmokers, phenotypically normal smokers, and individuals with COPD.

Rationale: Neuroendocrine cells (NEC), present in small numbers (0.4%) throughout the airway epithelium, have been implicated in the pathogenesis of small cell lung cancer (SCLC) and some non-small cell lung cancers (NSCLC). Based on the knowledge that cigarette smoking is associated with hyperplasia of NEC, it was hypothesized that cigarette smoking up-regulates the gene expression profile of NEC.

Methods: Affymetrix microarrays were used to evaluate gene expression in airway epithelium obtained by bronchoscopy from large and small airways including 31 samples from 15 nonsmokers, 53 samples from 27 phenotypically normal smokers, and 6 samples from 6 individuals with COPD, with a focus on genes known to be expressed by neuroendocrine cells, including ubiquitin C-terminal hydrolase-L1 (UCHL 1), bombesin, calcitonin, neuron specific enolase and chromogranin A and C.

Results: Consistent with previous studies showing increased levels of bombesin in lavage samples of smokers, bombesin gene expression was up-regulated in airway epithelium of smokers compared to nonsmokers (2.7-fold, p<0.03). Interestingly, UCHL1, a gene that is overexpressed in SCLC and NSCLC, was significantly up-regulated in smokers compared to non-smokers (9.9-fold, p<0.005). UCHL1 was expressed in 83% of smokers and 100% of COPD samples. Expression of other NEC specific markers was not increased in smokers. There was no significant difference in expression level of any neuroendocrine gene between smokers and individuals with COPD (p>0.1).

Conclusions: UCHL1 hydrolyzes the ubiquitin monomer from proteins that targets them for degradation, and plays a role in cell cycle regulation, proliferation, and apoptosis. In this context, its up-regulation in smokers may represent an early marker of cigarette smoke induced lung injury and failure to up-regulate UCHL1 may serve to indicate a future risk of malignancy.

### Example 8

This example compares the expression of clade B, member 3, and serine/threonine kinase 17b in nonsmokers and healthy smokers.

Rationale: The pathogenesis of the inflammatory response observed in large (LA) and small (SA; 4th to 12th generation airway branching) lung airways in COPD is not well elucidated. It was hypothesized that (1) gene expression profiling of lung airway epithelial cells (AEC) from SA and LA in healthy smokers (SM) and nonsmokers (NS) will identify genes relevant to COPD pathogenesis; (2) healthy SM display a different expression profile in SA and LA, compared to NS; (3) cigarette smoking induces a differential expression profile in AEC from SA vs. LA.

Methods: To test these hypotheses, transcriptional profiling studies were initiated in AEC from SA and LA of healthy SM and NS. Ten subjects were evaluated: 6 NS (4 males, 2 females), 4 SM (3 males, 1 female), 22+3 pack-yr; ages 29 to 48. AEC from the LA and SA were obtained by fiberoptic bronchoscopy with brushings, and RNA was extracted and then analyzed using an Affymetrix HG133A array.

Results: SA had an increased number of ciliated cells in SM [77.9+4.4% (SA) vs. 43+1.9% (LA), p<0.001] and NS [75+5.4% (SA) vs. 52+2.4% (LA), p=0.01]; and a decreased number of basal cells in SM [6.8+0.5% (SA) vs. 24+1.7% (LA), p<0.001] and NS [6.6+1.5% vs. 20.8+2.7%, p<0.002]. Consistent with SA sampling, surfactant genes were up-regulated in AEC of SA in SM and NS (p<001). SM up-regulated 103 genes in SA and LA, in various categories; for example, members of the cytochrome P450 family (xenobiotic/detoxification) were up-regulated>10 fold (p<0.001). Interestingly, SA from SM demonstrated down-regulation of the serine proteinase inhibitor clade B, member 3, and serine/threonine kinase 17b, an inducer of apoptosis, (p=0.002).

Conclusion: Expression profiling studies will play a key role in establishing the role of the SA in COPD pathogenesis.

### Example 9

This example compares the expression of mucin genes in an experimental lung injury model.

Rationale: Mucins, which are protein cores with complex carbohydrate side chains, are produced by the airway epithelium as part of the airway defenses. Depending on their structure, airway mucins are either secreted to become part of the mucociliary escalator, or tethered to epithelial cells to function locally.

Methods: Brush-induced injury of the airway epithelium of phenotypically normal individuals was used to help understand the pattern of expression of the mucin genes expressed by the human airway epithelium in response to injury. Mucin gene expression was evaluated in a total of 10 airway epithelium samples obtained by fiberoptic bronchoscopy and brushing from 5 phenotypically normal subjects, using Affymetrix HG-U133 Plus 2.0 microarrays. The response to injury was evaluated by brushing the same site at day 0, and again at day 7. The relative contribution of each of the 19 mucin genes represented in the HG-U133 Plus 2.0 array to total mucin gene expression was quantified.

Results: In the resting state, the airway epithelium expressed 10 mucin genes in >50% (n=6) samples; MUC1, 2, 3A, 4, 5AC, 5B, 13, 15, 16, and 20, with MUC 2 and 15 showing the highest % expression and MUC16 the lowest % expression. As a group, the secreted mucins (MUC 2, 5AC, 5B) represented 37% of the total mucin gene expression, and the tethered mucins (MUC 1, 3A, 4, 13, 15, 16 and 20) represented 63%. The gene expression levels of 3 mucin genes were significantly changed post-injury; expression of the tethered mucins MUC 1 and 16 increased 1.5-fold at day 7 (both p<0.01), while another tethered mucin, MUC 15, decreased 2.0-fold (p<0.04).

Conclusions: These observations suggest that there are differences in the response to injury of the mucin genes expressed in the human airways, an observation that has implications for understanding of the disarray in mucus production in chronic diseases such as COPD.

### Example 10

This example compares the expression of Notch pathway genes in nonsmokers, healthy smokers, and individuals with COPD.

Rationale: Abnormal airway epithelial differentiation is characteristic of chronic obstructive pulmonary disease (COPD). Given that the Notch signaling pathway plays a central role as a “gatekeeper” to epithelial differential in lung morphogenesis, it was hypothesized that Notch signaling is deranged in COPD, thereby contributing to abnormal epithelial differentiation.

Methods: Pure samples of airway epithelial cells were obtained via bronchoscopy and airway brushing of nonsmokers (n=5), healthy smokers (n=10), and individuals with COPD (n=6), and Affymetrix microarray technology was used to assess the expression of 54 genes in the Notch pathway that control whether differentiation is inhibited (“Notch on”) or permitted (“Notch off”).

Results: 35 genes in the Notch pathway were expressed in >60% of airway epithelial samples. To test whether Notch is central to human airway epithelial differentiation, healthy individuals underwent repeat assessment 7 days after brush denuding of the epithelium as forced models of regeneration/differentiation. As expected, after injury there was evidence of “Notch off,” with a 16-fold increase in expression of Hes6 (p<0.01), an antagonist of Notch signaling, consistent with loss of inhibition of differentiation. Importantly, at rest compared to smokers, individuals with COPD overexpressed (>1.5-fold) Notch1 and Notch3, genes coding for the Notch receptor, as well as Jagged1, a ligand (all p<0.05), which changes promote maintenance of undifferentiated cells.

Conclusions: The Notch pathway is expressed in the human airway epithelium and is turned off, favoring differentiation, during repair following airway epithelial injury. The increased Notch expression in COPD (suppressing differentiation) suggests that this pathway plays a role in the derangements of epithelial differentiation observed in this disease.

### Example 11

This example compares the expression of C-X3-C motif ligand 1, and C-X-C motif ligand 3, IL-16, BCL2-associated transcription factor 1, and MUC15 genes in healthy nonsmokers, healthy smokers, and individuals with early small airway disease.

Rationale: The small airways (SA) are the main site of disease in individuals with smoking-related COPD. Healthy smokers have evidence of SA inflammation despite normal lung function. It was hypothesized that in smokers, the progression from the healthy to the COPD phenotype is preceded by alterations in SA gene expression relevant to the COPD pathogenesis.

Methods: Affymetrix microarray chips to were used to assess gene expression of SA epithelium obtained by fiberoptic bronchoscopy and brushing of 10th-12th order bronchi from 5 healthy nonsmokers, 11 normal smokers, 6 smokers with early evidence of SA disease (FEV1/FVC>70%, but decreased DLCO), and 4 individuals with established COPD.

Results: Normal smokers up- and down-regulated 25 genes in 7 categories relevant to COPD pathogenesis (cytokines/innate immunity, apoptosis, pro-fibrosis, mucin, responses to oxidants and xenobiotics, antiproteases, and general cellular processes). For example, the interferon receptor 2 gene, a type 1 interferon receptor subunit, was up-regulated in smokers vs. non smokers (p<0.02). The genes (C-X3-C motif) ligand 1 and (C-X-C motif) ligand 3, important in T lymphocytes recruitment, were down-regulated in smokers vs. non smokers (p<0.04). Individuals with early SA disease differentially expressed genes important in COPD pathogenesis; for example the IL-16 gene, important in lymphocyte recruitment, the BCL2-associated transcription factor 1 gene, important in apoptosis, and the MUC15 gene, important in mucus production, were up-regulated in individuals with early SA disease vs. normal smokers (p<0.03).

Conclusion: In the context that COPD starts in the SA, assessment of alterations in SA epithelium early in the disease process are important for understanding the pathogenesis of COPD and should reveal new therapeutic targets.

### Example 12

This example compares the expression of TNF-α and IFN-γ inducible genes in normal nonsmokers, normal smokers, and asymptomatic HIV-1+ smokers.

Rationale: HIV-1 positive smokers with no evidence of opportunistic infections have an increased incidence, accelerated progression, and lower pack-yr threshold for the development of emphysema than the general population. It was hypothesized that the alveolar macrophage (AM) plays a central role in the pathogenesis of accelerated emphysema in HIV-1+ smokers, by alteration of gene expression to a pattern that mediates emphysema, and that this pattern reflects influence of tumor necrosis factor-α (TNF-α) and interferon-γ (IFN-γ), mediators up-regulated in HIV-1+ individuals and linked to the pathogenesis of emphysema.

Methods: AM were obtained by bronchoalveolar lavage from 3 groups: normal nonsmokers, normal smokers, and asymptomatic HIV-1+ smokers (all with CT evidence of mild emphysema and DLCO 65%+3% predicted). Relative gene expression was evaluated in purified AM using Affymetrix microarray (HG-U133 Plus 2) normalized per gene across all samples.

Results: Overall, comparison of normals to HIV+ showed that relative expression of individual AM genes normalized per array was similar (r2=0.93), but there were many outliers. Of the 96 genes that met criteria of >2-fold up-regulated and p<0.05 for between-group differences, 25 were TNF-α inducible, and 10 were IFN-γ inducible. When the data were mined for AM genes relevant to the pathogenesis of emphysema, the overall relative expression matrix metalloproteinases-1, -9, and -12 was increased in HIV+ smokers (p<0.05, ANOVA), as was expression of chemotactic factors monocyte chemoattractant protein-1/CCL2, macrophage inflammatory protein-1α/CCL3, and interleukin-8 (p<0.05, ANOVA).

Conclusions: These data suggest that up-regulated expression of AM genes in HIV-1+ smokers may promote accelerated emphysema. This process may be driven in part by TNF-α and INF-γ in the milieu of the alveolus.

### Example 13

This example compares the expression of axonemal genes, EML1, and ERRBL1 in an experimental model of lung injury.

Rationale: Ciliated cells of the airway epithelium (AE) transport mucus to remove inhaled irritants and infectious particles. To identify novel genes in the cilia-related transcriptome, a putative list of genes was identified by combining a screen of genes expressed in the normal human AE with an evolutionary-wide genomic analysis of conserved cilia-related genes. To test that these novel genes were cilia-related, expression levels were compared during ciliogenesis following brush-induced injury to the AE.

Methods: An experimental injury model was performed in healthy volunteers (n=5) in which the AE was sampled at days 0 (baseline), 7, and 14 to evaluate changes in cilia-related gene expression assessed by Affymetrix microarray in a forced model of ciliogenesis.

Results: From the genomic analysis, 64 genes were identified that represent the human airway cilia-related transcriptome. Following injury, there was significant decrease at day 7 in the percentage of ciliated cells recovered (45% vs. 23%, p<0.01) and in cilia length (7.8 μm vs. 5.2 μm, p<0.05) that returned to near baseline levels at day 14. The gene expression levels for several well characterized axonemal genes (dynein heavy chains 5 and 7, and light chain 1) were highly correlated with the % of ciliated cells during regeneration (p<0.01 for all genes). From the cilia-related transcriptome, several novel genes [echinoderm microtubule associated protein 1 (EML1), estrogen-related receptor β-like 1 (ERRBL1), and whole domain repeat 10 (WDR10)] were identified, the expression levels of which also correlated with the percentage of ciliated cells present during regeneration (p<0.05 for all genes).

Conclusions: These studies establish a strategy for identifying several novel genes involved in the ciliogenesis-related transcriptional response of the human AE in vivo.

### Example 14

This example demonstrates the differential expression of genes in healthy nonsmokers versus healthy smokers.

Normal nonsmokers and normal current cigarette smokers were studied. Individuals were determined to be phenotypically normal based on standard history, physical exam, complete blood count, coagulation studies, liver function tests, urine studies, chest X-ray, EKG, and pulmonary function tests. To verify smoking status, a complete smoking history was obtained, urine samples were evaluated for nicotine and cotinine, and venous blood was evaluated for carboxyhemoglobin. A total of 33 individuals participated in the study. All individuals (nonsmokers and smokers) had no prior medical history, and their physical exams were normal. All were HIV negative and had normal blood (hematology, coagulation, biochemistry, and α1-antitrypsin) and urine parameters. All chest X rays and pulmonary function tests (spirometry, lung volumes, and diffusion capacity) were normal. The 16 normal smokers had a 25±7 pack-year smoking history, actively smoking 1.0±0.3 pack per day. Urine nicotine and cotinine and venous carboxyhemoglobin levels verified the smoking status of these individuals. In the group of nonsmokers, negative urine nicotine and cotinine were consistent with a negative smoking history. There were no differences among age (p>0.2), sex (p>0.6), or race (p>0.7) among the smokers and nonsmokers (see Table 1).

TABLE 1

| | Group A¹ | | Group B¹ | |
|---|---|---|---|---|
| Parameter | Non-smokers | Smokers | Non-smokers | Smokers |
| n | 5 | 6 | 12 | 10 |
| Sex (male/female) | 3/2 | 4/2 | 10/2 | 7/3 |
| Age (yr) | 34 ± 5 | 39 ± 5 | 42 ± 8 | 44 ± 4 |
| Race (B/W/H)² | 2/2/1 | 2/3/1 | 6/4/2 | 5/5/0 |
| Smoking history (pack-yr) | 0 | 24 ± 4 | 0 | 25.8 ± 9 |
| Smoking habits (pack per day) | 0 | 1.0 ± 0.2 | 0 | 0.95 ± 0.4 |
| Urine nicotine (ng/ml)³ | 1.8 ± 4.0 | 646 ± 502 | <10 | 514 ± 635 |
| Urine cotinine (ng/ml)³ | 27 ± 6 | 1251 ± 870 | <40 | 1381 ± 619 |
| Venous carboxyhemoglobin (%)⁴ | N.D.⁶ | 5 ± 0.3 | 0.9 ± 0.21 | 3.03 ± 0.6 |
| Pulmonary function parameters⁵ | | | | |
| FVC | 111 ± 10 | 109 ± 18 | 105 ± 9 | 103 ± 13 |
| FEV1 | 108 ± 12 | 100 ± 17 | 105 ± 7 | 101 ± 13 |
| FEV1/FVC | 95 ± 5 | 91 ± 4 | 82 ± 6 | 80 ± 5 |
| TLC | 100 ± 5 | 103 ± 14 | 97 ± 8 | 96 ± 14 |
| DLCO | 93 ± 2 | 91 ± 9 | 95 ± 12 | 94 ± 10 |
| Cough, shortness of breach, sputum⁶ | No | No | No | No |
| Affymetrix chip used | HG-U133A | HG-U133A | HG-U133 Plus 2.0 | HG-U133 Plus 2.0 |

¹Demographic characteristics of 33 individuals in the study. The demographics represents two sets of independent study individuals. Group A includes 11 healthy individuals, 5 healthy non-smokers and 6 healthy smokers in whom small airway epithelial gene expression was assessed with the Affymetrix HG-U133A gene chip. Group B includes 22 healthy individuals (12 non-smokers and 10 smokers) in whom small airway epithelial gene expression was assessed with the Affymetrix HG-U133 Plus 2.0 gene chip. Data are presented as mean ± standard deviation.
²B = Black; W = White; H = Hispanic.
³Urine nicotine and cotinine used as screen to insure current smoking; >200 = active smoker, 50-200 = passive smoker; <50 non-smoker. Data represents the mean of two determinations from the day of the initial screening and day of bronchoscopy. A new assay was used for part B; with the new assay, undetectable levels are considered <10 ng/ml for nicotine and <40 ng/ml for cotinine.
⁴Determined for all smokers (Groups A and B) and in all non-smokers in Group B; venous carboxyhemoglobin was used as a secondary marker of current smoking; non-smokers <1.5%. Data presented as mean ± standard error.
⁵FVC, forced vital capacity; FEV1, forced expiratory volume in 1 sec; TLC, total capacity; DLCO, total diffusion capacity, FVC, FEV1, TLC and DLCO are presented as percent predicted; FEV1/FVC is expressed as percent observed.
⁶Symptoms of cough, shortness of breath and sputum production.
⁷For Group A, the HG-U133A chip was used (including probes representing approximately 22,000 full-length human genes); for Group B of the study, the HG-U133 Plus 2.0 chip was used (probes representing the entire human genome).

Sampling the Airway Epithelium. Fiberoptic bronchoscopy was used to collect airway epithelial cells. After mild sedation was achieved with demerol and versed, and routine anesthesia of the vocal cords and bronchial airways with topical lidocaine, the fiberoptic bronchoscope (Pentax, EB-1530T3) was positioned distal to the opening of the desired lobar bronchus. To obtain small airway epithelial cells, a 2 mm diameter brush was advanced approximately 7 to 10 cm distally from the 3rd order bronchial branching under fluoroscopic guidance. The distal end of the brush was wedged at about the 10th to 12th generation branching of the right lower lobe, and small airway epithelial cells were obtained by gently gliding the brush back and forth on the epithelium 5 to 10 times in 10 different locations in the same general area. The cells were detached from the brush by flicking into 5 ml of ice-cold bronchial epithelial basal cell medium (BEBM, Clonetics, Walkersville, Md.). An aliquot of 0.5 ml was used for differential cell count and to develop slides for immunohistochemistry studies (typically 2×10⁴ cells per slide). The remainder (4.5 ml) was processed immediately for RNA extraction. To compare cell types obtained from sampling the small airways to the cell types obtained from brushing the large airways, samples of the large airway epithelium were obtained in the same individuals using 2.0 mm disposable brushes to sample the epithelium of 2nd and 3rd order bronchi in the right lower lobe as previously described (Hackett, Am. J. Respir. Cell. Mol. Biol., 29: 331-343 (2003); Kaplan, Cancer Res, 63: 1475-1482 (2003)).

Morphology of Airway Epithelial Cells. The total number of cells recovered by bronchial brushing from the airways was determined by counting on a hemocytometer. To quantify the percentage of epithelial and inflammatory cells and the proportions of ciliated, basal, secretory, and undifferentiated epithelial cells, aliquots of 2×10⁴ cells were prepared by centrifugation (Cytospin 11, Shandon Instruments, Pittsburgh, Pa.), and the cells were assessed by staining with Diff-Quik (Dade Behring, Newark, N.J.). Aliquots were also assessed by immunohistochemistry with antibodies directed against surfactant protein A (SPA, Lab Vision Corporation, Fremont, Calif.) and Clara cell protein 10 (CC10; Bio Vendor, Candler, N.C.). Cytospin preparations were fixed with 4% paraformaldehyde in phosphate buffered saline, pH 7.4 (PBS), for 20 min at 23° C. Incubation with anti-SPA and anti-CCIO was carried out overnight at 4° C.; subsequently cytospins were washed with PBS, followed by incubation with a secondary peroxidase-coupled antibody for 30 min at 23° C. The final step included incubation with a 3,3′-diaminobenzidine chromogenic substrate detection system (Dako, Carpentaria, Calif.), which rendered positive cells brown. All cytospins were counterstained with hematoxylin. Species and subtype-matched antibodies were used as negative controls.

To assess the cell populations by transmission electron microscopy, the brushed airway epithelial cells were suspended in BEBM medium and then pelleted at 2000 rpm using a Beckman GH 3.8 rotor in a Beckman GS-6R tabletop centrifuge at 900 g. The medium was gently aspirated, and the pellet was overlaid with fixative containing 2.5% glutaraldehyde (Electron Microscopy Sciences, Hatfield, Pa.), 4% paraformaldehyde (Electron Microscopy Sciences), and 0.02% picric acid (Sigma Chemical Company, St. Louis, Mo.) prepared in 0.1 M sodium cacodylate buffer, pH 7.3, and maintained at 23° C. After fixation, pellets were rinsed 3× with 0.1 M sodium cacodylate buffer and post-fixed in 1% OsO4 and 1.5% potassium ferricyanide for 60 min at 23° C. Cell pellets were washed 3× in 0.1 M sodium cacodylate buffer and stained en bloc with 1.5% uranyl acetate for 30 min at 23° C. Samples were dehydrated using a graded ethanol series. After a final dehydration step in 100% ethanol, samples were infiltrated in a 1:1 mixture of ethanol and Spurr's resin followed by infiltration and embedding in Spurr's resin (Electron Microscopy Sciences). Sections were cut at 60 to 65 nm (silver-gold) using a Diatome diamond knife (Diatome, Hatfield, Pa.) on a Leica Ultracut S (Leica Microsystems, Bannockburn, Ill.). Sections were contrasted with lead citrate and viewed on a JSM 100 CX-II electron microscope (JEOL, Peabody, Mass.) operated at 80 kV. Images were recorded on Kodak 4489 Electron Image film (Electron Microscopy Sciences) and then digitized on an Epson Expression 3200 Pro scanner at 800 dpi (Epson America, Long Beach, Calif.).

RNA and Microarray Processing. The HG-U133A and the HG-U133 Plus 2.0 arrays (Affymetrix, Santa Clara, Calif.), including probes representing ˜22,000 and ˜39,000 full-length human genes respectively, were used to evaluate gene expression. Total RNA was extracted using TRIzol® (Invitrogen, Carlsbad, Calif.), yielding 2 to 4 μg from 106 cells. Quality control includes an A260/A280 ratio of 1.7 to 2.3. First and second strand cDNA were synthesized from 6 v of RNA using the Superscript II kit (Invitrogen). The biotinylated RNA transcript was produced using the BioArray HighYield reagents (Enzo, New York, N.Y.), purified by the RNeasy kit (Qiagen, Valencia, Calif.), and fragmented immediately before use. Hybridization to test chips and microarrays were performed according to Affymetrix protocols. The quality of the RNA labeling was verified by hybridization to a test chip, and only test chips with a 3′ to 5′ ratio of <3 were deemed satisfactory. Samples passing the quality control criteria were then hybridized to the HG-U133A or the HG-U133 Plus 2.0 array, processed by the fluidics station to receive the appropriate reagents/washes, and then transferred to the scanner for duplicate scanning. The captured image data for HG-U133A arrays was processed using the Affymetrix Microarray Suite version 5 (MAS5) algorithm. Image data from the HG-U133 Plus 2.0 arrays was processed using MAS5 and also by the Robust Multi-array Average (RMA) algorithm. Irizarry, Biostatistics 4: 249-264 (2003), using GENESPRING version 6.2 software (Agilent technologies). MAS5 takes into account the perfect match and the mismatch values, while the RMA method utilized only the perfect match values. MAS5-analyzed data was normalized using GENESPRING as follows: (1) per array, by dividing the raw data by the 50th percentile of all measurements, and (2) per gene, by dividing the raw data by the median of the expression level for the gene in all samples. RMA pre-processed data was normalized to the median measurement for the gene across all the arrays in the data set, since the per array normalization step is included in this method.

Gene Expression in the Small Airway Epithelium in Normal Nonsmokers. In order to determine the normal gene expression profile (the normal transcriptome) of the small airway epithelium in healthy nonsmokers, RNA from the small airway epithelium of healthy nonsmokers was assessed for gene expression with the HG-U133 Plus 2.0 microarray. Expressed was defined as having an Affymetrix Detection Call of Present in >50% of the samples. A total of 27,244 probe sets were grouped into functional categories, using the database from the Affymetrix NetAffx Analysis Center by the Gene Ontology (GO) Biological Processes classification. Of these, 10,935 probe set IDs were classified as unknown function, and were not used to generate the data on the distribution of types of genes expressed. The remaining genes were classified in the general biological processes categories.

Genes Up- and Down-regulated in the Small Airway Epithelium of Phenotypically Normal Smokers Compared to Normal Nonsmokers. Initial assessment of differentially expressed genes in small airway epithelium of smokers compared to nonsmokers was carried out in 11 healthy individuals (5 nonsmokers and 6 smokers, for convenience referred to as part A of the study). To identify the categories of small airway epithelial genes up- and down-regulated by smoking in these individuals, and to provide an overview of the relative-fold changes of these genes by gene category relevant to the pathogenesis of COPD, microarray analysis was carried out using the Affymetrix HG-U133A microarray. Genes were considered significant if p<0.05 and the fold-change was >2-fold between the two groups. The fold-change was calculated by dividing the average expression value in all smoker samples by the average expression value in nonsmoker samples. The genes were categorized according to the Gene Ontology annotations (GO), in categories relevant to COPD pathogenesis, as well as additional general categories, such as signal transduction and transcription. The data were expressed as “+” for up-regulated genes and “−” for down-regulated genes.

Based on the assessment of patterns of gene expression in small airways of healthy smokers vs. healthy nonsmokers showing altered gene expression patterns, and from the data in the literature regarding molecular pathways in airway epithelium previously implicated in the pathogenesis of COPD, a list of categories was generated of genes expressed in the small airway epithelium relevant to the pathogenesis of COPD, including cytokines/innate immunity, apoptosis, profibrotic, mucin, response to oxidants, antiproteases, and general cellular processes. From the preliminary data comparing genes up- and down-regulated in the small airway epithelium of smokers to nonsmokers in the first 11 individuals studied, a total of 152 genes with known function were identified and placed into the various categories. From this catalog of genes, 1 to 8 genes were chosen as examples in each category.

To confirm the initial gene list categories relevant to the pathogenesis of COPD generated by assessment of differential gene expression in small airway epithelium in the first 11 healthy individuals studied (group A, 6 smokers vs. 5 nonsmokers), the small airway epithelial gene expression was independently assessed in an entirely new group of healthy individuals (n=22; 10 healthy smokers and 12 healthy nonsmokers; referred to as group B) who shared similar phenotypic characteristics as the initial 11 individuals studied in group A. Assessment of the small airway epithelium gene expression of these new 10 healthy smokers vs. 12 healthy nonsmokers (group B) was carried out with a newest generation Affymetrix chip, the HG-U133 Plus 2.0.

As detailed above, data from the HG-U133 Plus 2.0 arrays from small airway epithelium of individuals in group B were processed using MAS5 and also independently by the RMA algorithms. Genes were considered significant if p<0.05, and the fold-change (up- or down-regulation) was >1.5-fold-between the two groups in both the MAS5 and the RMA-generated datasets. To limit the number of false positives, the Benjamini and Hochberg false discovery rate multiple test correction was applied to both the MAS5 and the RMA-generated datasets (Benjamini, J. R. Stat. Soc., 857: 289-300 (1995)). Fold-change was calculated by dividing the geometric mean expression value in all smoker samples by the geometric mean expression value in nonsmoker samples. Similar to the assessment of gene expression in group A, the genes differentially expressed in group B were classified according to categories relevant to COPD pathogenesis as described above. This list of genes in response to cigarette smoking in healthy individuals, generated from the analysis of groups A and B, are referred to as the “small airway epithelial smoking-induced phenotype” in healthy individuals. This gene list is far from complete but, taking into account all of the available information, provides a representative picture of the modifications of gene expression of the small airway epithelium in smoking relevant to the pathogenesis of COPD.

Cluster Analysis. Unsupervised classification of samples was carried out by hierarchical cluster analysis, by gene and by sample, using the standard correlation, with the GENESPRING software (Agilent Technologies), using the expression levels of the genes (up-regulated and down-regulated) modulated by smoking obtained by assessment of gene expression in group B, using the GENESPRING clustering function (standard correlation) by individual sample and by gene. The goal was to obtain a graphical representation of general variability within this population.

TAQMAN RT-PCR. TAQMAN real-time RT-PCR was carried out for 8 nonsmokers and 8 smokers from group B, using the same RNA samples that had been used for the microarray analysis. First strand cDNA was synthesized from 2 μg of RNA in a 100 μl reaction volume, using the TAQMAN Reverse Transcriptase Reaction Kit (Applied Biosystems, Foster City, Calif.), with random hexamers as primers. The cDNA was diluted 1:100 or 1:50, and each dilution was run in triplicate wells. Five μl were used for each TAQMAN PCR reaction in 25 μl final reaction volume, using pre-made kits from Applied Biosystems. Relative expression levels were calculated using the AACt method (Applied Biosystems), using ribosomal RNA as the internal control (Human Ribosomal RNA Kit, Applied Biosystems), and the average value for nonsmokers as the calibrator. The rRNA probe was labeled with VIC, and the probes for the genes of interest were labeled with FAM. The PCR reactions were run in an Applied Biosystems Sequence Detection System 7500. The relative quantity (ΔΔCt) was determined using the algorithm provided by Applied Biosystems. For comparison purposes, the data for each individual were normalized to the median across all nonsmokers and smoker samples, as was done with the microarray data.

Non-microarray-related Statistical Analyses. Comparison of the percentage cell types and demographic parameters in the nonsmokers and smokers was performed by two-tailed Student's t-test. A two-way ANOVA with smoking status (smokers vs. nonsmokers) and method (microarray vs. TAQMAN) as independent factors was carried out using StatView v 5.0 (SAS Institute) to demonstrate that smoking was significant but methodology was not, thereby confirming the agreement between the two methodologies.

### Results

Study Population. The study individuals were divided into 2 groups (A and B; see Table 1). Airway epithelial samples from individuals in group A (n=1 1; 6 healthy smokers and 5 healthy nonsmokers) were used to establish the morphologic differences between large and small airway epithelium, to determine the presence of Clara cells in samples obtained from small airways, to demonstrate that airway epithelial cells from small airways but not the large airways expressed surfactant apoproteins-related genes, and to carry out preliminary assessment of the differences in gene expression among smokers compared to nonsmokers. The initial assessment of differential gene expression in small airway samples from healthy smokers vs. nonsmokers carried out in healthy individuals from group A was done with the Affymetrix HG-U133A microarray chip.

Small airway epithelium from individuals in group B (n=22; 10 healthy smokers and 12 nonsmokers) was used independently to confirm the differential gene expression in the various gene categories relevant to the pathogenesis of COPD which were initially found following assessment of gene expression in small airway epithelium of individuals from group A. Gene expression in airway samples from individuals in group B was assessed with the newest microarray chip, the Affymetrix HG-U133 Plus 2.0. Small airway epithelium RNA from individuals in group B was also used for TAQMAN RT PCR confirmation of a selected group of differentially expressed genes among smokers vs. nonsmokers.

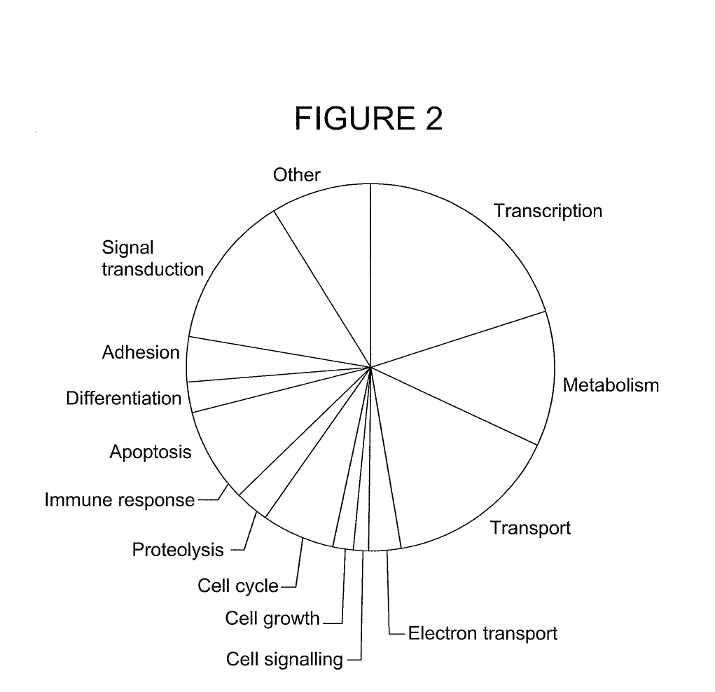

Genes Expressed in the Small Airway Epithelium of Normal Nonsmokers. In order to determine the normal pattern of gene expression of the small airway epithelium in healthy individuals not exposed to the insult of cigarette smoking, and to have a baseline “normal transcriptome” for comparison with the expression in small airway epithelium from smokers, the small airway epithelium RNA of 12 healthy nonsmokers from group B was assessed with the HG-U133 Plus 2.0 microarray. Of the total 54,675 probe sets represented in the HG-U133 Plus 2.0 microarray, 27,244 were “present” or expressed according to the MAS5 algorithm in >50% of the samples. These genes were functionally grouped into 14 different categories. The largest categories were transcription, transport, metabolism, signal transduction, followed by cell cycle, apoptosis, and cell adhesion; other categories included differentiation, immune response, proteolysis, electron transport, cell growth, and cell signaling-related genes (FIG. 2).

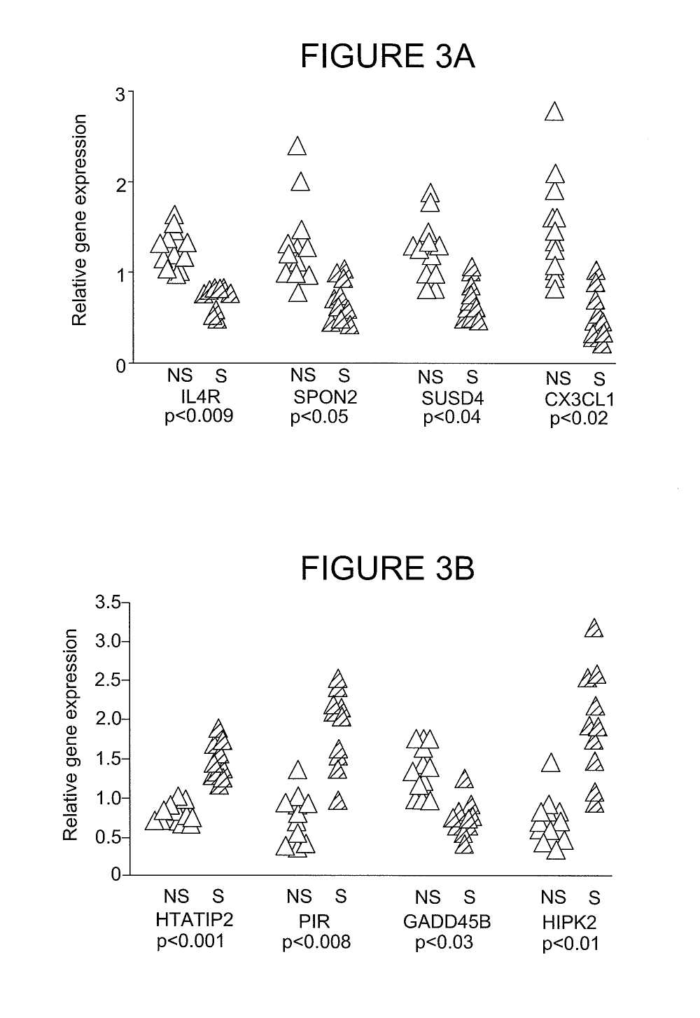

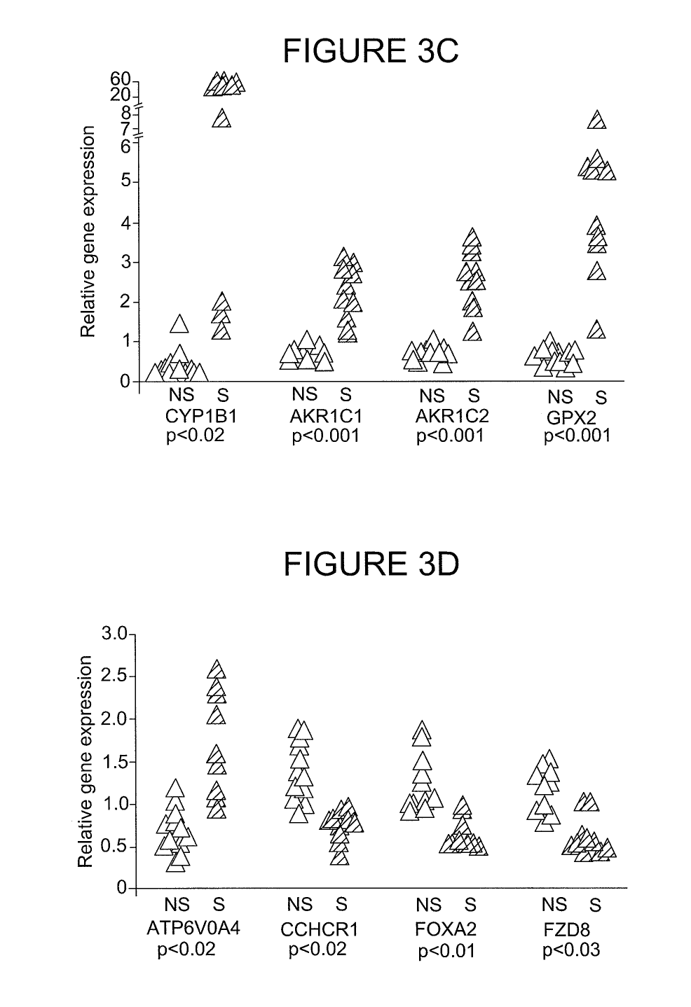

Genes Up- and Down-regulated in the Small Airway Epithelium of Phenotypically Normal Smokers Compared to Normal Nonsmokers. Relevant to the pathogenesis of COPD, assessment of gene expression in the small airway epithelium of smokers compared to nonsmokers showed a significant up- and down-regulation of several genes in various functional categories (Table 2, FIGS. 3A-D and 4). Initial assessment of gene expression in small number of individuals (group A, n=11, 6 smokers vs. 5 nonsmokers) demonstrated a total of 103 genes up-regulated and 49 genes down-regulated in several functional categories in the small airway epithelium of phenotypically healthy smokers compared to nonsmokers. Of these 152 genes, 133 genes were of known function and were grouped into biologically relevant categories. Based on the assessment of the small airway gene expression and a review of the molecular pathways shown in the literature to be related to the pathogenesis of COPD, the most relevant 6 of these categories were chosen to generate a representative “small airway epithelial smoking-induced phenotype.” These categories included cytokine/innate immunity, apoptosis, pro-fibrotic, response to oxidants and xenobiotics, antiproteases, and general cellular processes.

TABLE 2

| | | | | | Group B¹ | | | |
|---|---|---|---|---|---|---|---|---|
| | | | Group A¹ | | S vs NS | | S vs NS | |
| | | | S vs NS- | | fold | p value | fold | P value |
| | | Gene | fold | p value | change | S vs NS | change | S vs NS |
| Category | Gene | symbol | change² | S vs NS³ | RMA² | RMA⁴ | MAS5⁴ | MAS5⁵ |
| Cytokine/ | chemokine (C- | CX3CL1 | −2.97 | <0.040 | 2.89 | <0.016 | −2.96 | <0.004 |
| innate | X3-C motif) | | | | | | | |
| immunity | ligand 1 | | | | | | | |
| Apoptosis | pirin | PIR | 2.78 | <0.001 | 2.64 | <0.007 | 2.22 | <0.028 |
| | growth arrest | GADD45B | −2.25 | <0.043 | −1.83 | <0.024 | −2.26 | <0.014 |
| | and DNA- | | | | | | | |
| | damage- | | | | | | | |
| | inducible, beta | | | | | | | |
| Response | cytochrome | CYP1B1 | 17.69 | <0.001 | 20.73 | <0.039 | 54.7 | <0.004 |
| to oxidants | P450, family 1, | | | | | | | |
| and | subfamily B, | | | | | | | |
| xenobiotics | polypeptide 1 | | | | | | | |
| | aldo-keto | AKR1B10 | 11.73 | <0.001 | 24.84 | <0.003 | 20.76 | <0.002 |
| | reductase | | | | | | | |
| | family 1, | | | | | | | |
| | member B10 | | | | | | | |
| | aldehyde | ALDH3A1 | 6.63 | <0.001 | 75.6 | <0.001 | 4.96 | <0.001 |
| | dehydrogenase | | | | | | | |
| | 3 family, | | | | | | | |
| | member A1 | | | | | | | |
| | alcohol | ADH7 | 6.24 | <0.001 | 7.21 | <0.001 | 6.1 | <0.001 |
| | dehydrogenase 7 | | | | | | | |
| | glutathione | GPX2 | 5.16 | <0.001 | 2.69 | <0.009 | 3.73 | <0.001 |
| | peroxidase 2 | | | | | | | |
| | NAD(P)H | NQO1 | 4.41 | <0.001 | 3.37 | <0.001 | 3.38 | <0.001 |
| | dehydrogenase, | | | | | | | |
| | quinone 1 | | | | | | | |
| | aldo-keto | AKR1C3 | 3.09 | <0.001 | 2.6 | <0.01 | 2.32 | <0.014 |
| | reductase | | | | | | | |
| | family 1, | | | | | | | |
| | member C3 | | | | | | | |
| General | ubiquitin | UCHL1 | 11.75 | <0.001 | 15.85 | <0.002 | 31.07 | <0.001 |
| cellular | carboxyl- | | | | | | | |
| processes | terminal | | | | | | | |
| | esterase L1 | | | | | | | |

¹Group A includes 11 healthy individuals, 5 healthy non-smokers and 6 healthy smokers in whom small airway epithelial gene expression was assessed with the Affymetrix HG-U133A gene chip. Group B includes 22 healthy individuals (12 non-smokers and 10 smokers) in whom small airway epithelial gene expression was assessed with the Affymetrix HG-U133 Plus 20 gene chip; for group B, expression values were independently generated using Robust Multiarray Average (RMA) and Microarray Suite 5 (MAS5). Genes were considered expressed when they had Affymetrix Present “P” calls in >50% of any given group of samples (non-smokers) in both group A and group B study individuals.
²Smokers (S) vs non-smokers (NS) fold change was calculated by dividing the average expression value in the smokers by the average expression value in the non-smokers.
³p values were calculated using the Welch t test (assuming unequal variances) using the Affymetrix HG-U133A gene chip; expression values were generated using MAS5.
⁴p values were calculated using the Welch t test (assuming unequal variances) using the Affymetrix HG-U133A Plus 2.0 gene chip; expression values were generated using RMA with Benjamini-Hochberg correction.
⁵Same as note 4 except expression values were generated using MAS5.

Following the initial assessment of differential gene expression in the 1st group of healthy individuals studied, these changes were verified by studying a larger group of healthy individuals (group B, n=22, 10 smokers vs. nonsmokers). Interestingly, consistent with the initial assessment using the HG-U133A chip (group A), genes in similar categories were differentially expressed in small airway epithelium of healthy smokers compared to nonsmokers assessed with the HG-U133 Plus 2.0 chip (group B; Table 2). The group B assessment, which was subject to a more rigorous analysis, demonstrated a more restricted number of genes up- or down-regulated [118 genes, 48 up-regulated and 70 down-regulated] compared to the initial gene list of 152 observed in the initial analysis of group A. The 118 genes differentially expressed in smokers vs. nonsmokers in group B included genes in the categories: cytokine/innate immunity, apoptosis, response to oxidants and xenobiotics, proteases/anti-proteases, and general cellular processes.

Following assessment of gene expression in small airway epithelium of healthy smokers vs. nonsmokers in groups A and B, a list was generated showing examples of genes differentially expressed in a similar fashion in smokers vs. nonsmokers in both groups A and B (Table 3). Although the list of genes in the different pathways does not represent the entire molecular signature in the pathogenesis of COPD, it represents early gene expression responses in the small airway epithelium of individuals at risk for the development of COPD.

In the context that the assessment of differential gene expression of healthy individuals in group B included a higher number of individuals (n=22; 10 smokers vs. 12 nonsmokers), with assessment of gene expression with the Affymetrix HG-U133 Plus 2.0, and a more rigorous analysis of the gene expression data which included independent assessment by RMA and MAS5 with Benjamini Hochberg correction, the following description of differentially expressed genes in the different categories relevant to the pathogenesis of COPD focuses on the results obtained from small airway epithelial cells from these individuals (Group B, Table 2, FIGS. 2-4).

Assessment of gene expression levels for the 118 genes modulated by smoking in the small airway epithelium of the study individuals in group B, using an unsupervised assessment by hierarchal cluster analysis, showed, as expected, clustering of the samples according to smoking status. This suggests that, taken as a group, similar changes are occurring among all healthy smokers. Likewise, as a group, healthy nonsmokers displayed a similar gene expression profile.

Cytokine/innate Immune Response-related Genes. Independent assessment of gene expression by RMA and MAS5 demonstrated that the small airway epithelium of smokers vs. nonsmokers down-regulated several immune-related genes. Down-regulated genes included the interleukin 4 receptor gene (p<0.002), which mediates many pro-inflammatory functions in human airways (Mueller, Biochem. Biophys. Acta., 1592: 237-250 (2002)), down-regulation of chemokine (C-X3-C motif) ligand 1 (p<0.02), also known as fractalkine, which is involved in cell adhesion and recruitment of monocytes and T lymphocytes cells (D'Ambrosio, Am. J. Respir. Crit. Care Med., 164: 1266-1275 (2001); Fujimoto, Am. J. Respir. Cell. Mol. Biol., 25: 233-238 (2001)), and down-regulation of spondin 2 (p<0.04) an activator of the Wnt/beta catenin signaling pathway, important in cell migration and proliferation and T cell development in the thymus (Kazanskaya, Dev. Cell., 7: 525-534 (2004); see also Table 2, FIG. 3A).

Apoptosis-related Genes. Consistent with prior studies demonstrating up-regulation of pirin, a pro-apoptotic gene, in the large airways of smokers (Kaplan, Cancer. Res., 63: 1475-1482 (2003); Spira, Proc. Natl. Acad. Sci. USA, 101: 10143-10148 (2004); Gelbman, Mol. Ther., 2:A803 (2005); Dechend, Oncogene, 18: 316-3323 (1999); Orzaez, Plant Mol. Biol., 46: 459-468 (2001); Wendler, J. Biol. Chem., 272: 8482-8489 (1997)), up-regulation of pirin was observed in the small airway epithelium of healthy smokers compared to nonsmokers (p<0.03; Table 2, FIG. 3B). Similarly, the pro-apoptosis-related genes HIV-Tat interactive protein 2, 30 kDa gene also known as TIP30, and the homeodomain interacting protein kinase genes (Hofmann, Cancer Res., 1:63:8271-8277 (2003); Shi, World J. Gastroenterol., 11: 221-227 (2005)), were up-regulated in smokers compared to nonsmokers (p<0.03). In contrast, the growth arrest and DNA-damage inducible, p-related gene, another pro-apoptotic gene (Shi, World J. Gastroenterol., 11: 221-227 (2005)), was down-regulated in small airway epithelium of healthy smokers (p<0.03).

Oxidative Stress and Xenobiotic-related Genes. Consistent with prior gene expression studies in large airways of phenotypically normal smokers (Hackett, Am. J. Respir. Cell Mol. Biol., 29: 331-343 (2003); Spira, Proc. Natl. Acad. Sci. USA, 101: 10143-10148 (2004)), assessment of oxidative stress and xenobiotic-related gene expression in the small airway epithelium of smokers compared to nonsmokers showed a significant up- and down-regulation of several genes with various functions (Tables 2, FIG. 3C). For example, the aldo-keto reductase family 1, member C1, and member C2 gene, the aldehyde dehydrogenase 3 family, member A1 gene, and the glutathione peroxidase 2 gene were significantly up-regulated in small airway epithelium of smokers compared to nonsmokers (p<0.002). Similarly, in the category of xenobiotics, the cytochrome P450, family 1, subfamily B, polypeptide 1 gene, was significantly up-regulated in the small airway epithelium of healthy smokers compared to nonsmokers (p<0.04).

General Cellular Processes Genes. Analysis of gene expression in small airway epithelium demonstrated up- and down-regulation of several genes involved in general cellular processes; for example, the ATPase H+ transporting, lysosomal V0 subunit a isoform 4, a gene involved in acidification of intracellular organelles for various intracellular processes such as protein sorting, receptor mediated endocytosis, and synaptic vesicle proton gradient generation, was up-regulated in healthy smokers (p<0.03). In contrast, the coiled-coil alpha-helical rod protein 1 (CCHCR1) gene, which is involved in metabolism and cell differentiation, the forkhead box A2 (FOXA2) gene, important in cell differentiation, and the frizzled homolog 8 (drosophila) FZD8 gene, involved in signal transduction were down-regulated (p<0.03; FIG. 3D, Table 2).

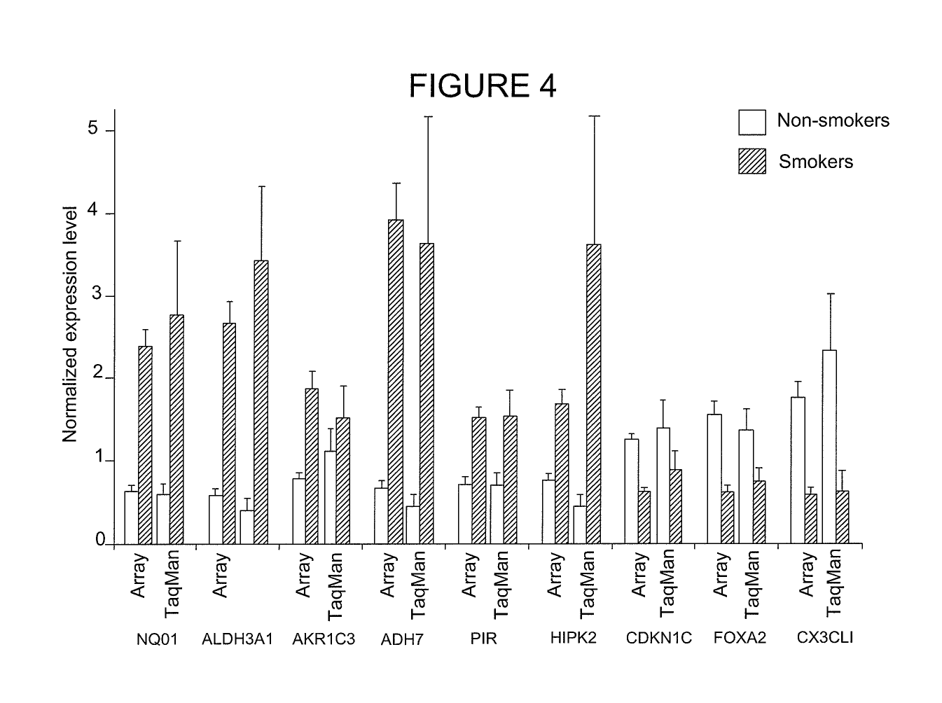

TAQMAN RT-PCR. Independent analysis of differentially expressed genes in small airway epithelium of smokers vs. nonsmokers in group B by real time quantitative TAQMAN RT-PCR confirmed the findings demonstrated by microarray assessment in a selected group of genes. The up-regulation of 4 genes involved in the response to oxidative stress or xenobiotics—namely the NAD(P)H dehydrogenase, quinone 1 (NQO1) gene; the aldehyde dehydrogenase 3 family, memberA1 (ALDH3A1) gene; the aldo-keto reductase family 1, member C3 (AKR1C3) gene, and the alcohol dehydrogenase 7 (ADH7) gene—and the down-regulation of 2 genes involved in apoptosis—namely pirin (PIR) and homeodomain interacting protein kinase 2 (HIPK2)—were confirmed by TAQMAN RT PCR. Similarly, the smoking-induced (a) down-regulation of the cyclin-dependent kinase inhibitor 1C (CDKN1C) gene, also known as p57 or Kip2, a cell cycle arrest protein, (b) down-regulation of the transcription factor forkhead box A2 (FOXA2) gene, involved in transcription of the surfactant genes and cell differentiation, and (c) down-regulation of the chemokine (C-X3-C motif) ligand 1 (CX3CL1) gene, an immune-related gene (FIG. 4) were confirmed. A two-way ANOVA with smoking status (smokers vs. nonsmokers) and method (microarray vs. TAQMAN) as independent factors confirmed that expression levels of these 9 genes were significantly affected by smoking status (p<0.05, all cases) and that method was not a significant factor (p>0.2, all cases).

### Example 15

This example compares the expression of pirin in healthy nonsmokers and healthy smokers.

Methods. Healthy nonsmokers and healthy chronic smokers were studied. The study individuals were part of an ongoing project to assess gene expression in the human airway epithelium in regard to the chronic airway disorders associated with cigarette smoking (Hackett, Am. J. Respir. Cell. Mol. Biol., 29: 331-343 (2003); Kaplan, Cancer Res., 63: 1475-1482 (2003); Heguy, Mol. Med., 9: 200-208 (2003)). The study was approved by the Weill Cornell Medical College Institutional Review Board and written informed consent was obtained from each individual before enrollment in the study. The smokers had an approximate smoking history of 20 pack-yr. and were in otherwise good health, with no evidence of respiratory tract infection, chronic bronchitis, or lung cancer. Each individual had to complete an initial screening evaluation, which included a history of smoking habits, respiratory tract symptoms, and prior illnesses, a complete physical exam, chest radiograph, and pulmonary function tests. Routine screening blood and urine studies were performed, including urinary levels of nicotine and its derivative cotinine, and serum levels of carboxyhemoglobin to verify reported levels of smoking.

Collection of Airway Epithelial Cells. All individuals who met the inclusion and exclusion criteria underwent fiberoptic bronchoscopy with brushing of the 3rd to 4th order bronchi as previously described (Hackett, Am. J. Respir. Cell. Mol. Biol., 29: 331-343 (2003)). Smokers were instructed not to smoke the evening before undergoing bronchoscopy. A 1 mm disposable brush (Wiltek Medical, Winston-Salem, N.C.) advanced through the working channel of the bronchoscope was used to collect the airway epithelial cells by gently gliding the brush back and forth on the airway epithelium 5 to 10 times in 10 different locations in the third branching of the bronchi in the right and left lower lobe of each individual. The cells were detached from the brush by flicking it into 5 ml of ice-cold LHC8 medium (GIBCO, Grand Island, N.Y.). An aliquot of 0.5 ml was kept for differential cell count and for cytology; the remainder (4.5 ml) was processed immediately for RNA extraction. Total cell number wad determined by counting on a hemocytometer. Differential cell count (epithelial vs. inflammatory cells) was assessed on cells prepared by cytocentrifugation (Cytospin 11, Shandon Instruments, Pittsburgh, Pa.) stained with DiffQuik (Baxter Healthcare, Miami, Fla.).

Preparation of cDNA and Hybridization to Microarray. All Analyses were performed using the Affymetrix HuGeneFL chip and associated protocol from Affymetrix (Santa Clara, Calif.). Total RNA was extracted from brushed cells using TRIzol (Life Technologies, Rockville, Md.) followed by RNeasy (Qiagen, Valencia Calif.) to remove residual DNA, which yielded approximately 2 μg RNA from 106 cells. First strand DNA was synthesized using the T7-(dT) (Hihara, FEBS Lett., 574: 101-105 (2004)), primer and converted to double-stranded cDNA using Superscript Choice system (Life Technologies). cDNA was purified by phenol chloroform extraction and precipitation, and the size distribution was examined after agarose gel electrophoresis. The cDNA was then used to synthesize biotinylated RNA transcript using the Bioarray High Yield reagents (Enzo, New York, N.Y.). This was purified by RNeasy (Qiagen) and fragmented immediately before use. The labeled cRNA was hybridized to the HuGeneFL GeneChip for 16 hr., and then processed by the fluidics station under the control of Microarray suite software (Affymetrix). The chip was then manually transferred to the scanner for data acquisition.

TAQMAN RT-PCR. RNA levels for pirin were measured relative to 18s rRNA by real time quantitative PCR (TAQMAN) with fluorescent TAQMAN chemistry using the ΔΔCt method (PE Biosystems, Instruction Manual). TAQMAN reactions for pirin were optimized and validated to show equal amplification efficacy compared to 18s rRNA using adult human lung RNA (Strategene, La Jolla, Calif.). Two sets of primers and probes were used, one to measure endogenous RNA (including 3′ untranslated end), and one to measure both endogenous and adenovirus-produced pirin mRNA (which spans two exons and would not amplify genomic DNA). The endogenous specific pirin primers were: forward AATGGGTTTGAAAGGGCCA [SEQ ID NO: 1] and reverse TCAAGACCTGCTCTTCCGCT [SEQ ID NO: 2], with probe AACCTGGAAATCAAAGATTGGGAACTAGTGGA [SEQ ID NO: 3]. The endogenous and adenovirus-produced pirin primers were: forward CACGCTGAGATGCCTTGCT [SEQ ID NO: 4] and reverse ACCATCTTCTCTGAGCTCCTCAA [SEQ ID NO: 5] with probe CAGCCCATGGCCTACAACTGTGGGTTATA [SEQ ID NO: 6].

Exposure of Primary Human Bronchial Epithelial Cells to Cigarette Smoke Extract. Three separate primary human bronchial epithelial (HBE) cell cultures were isolated from trachea and bronchi of donor lungs and seeded onto collagen-coated semi-permeable membranes (Millipore, Bedford, Mass.) and grown at the air-liquid interface (Karp, Methods Mol. Biol., 188: 115-137 (2002)). The viability of the cells was confirmed before each experiment by measurement of transepithelial resistance (Karp, Methods Mol. Biol., 188: 115-137 (2002)).

Cigarette smoke extract (CSE) was prepared using a modification of the method used by (Wyatt et al., Proc. Soc. Exp. Biol. Med., 225: 91-97 (2000)). Four research grade cigarettes (2R4F, University of Kentucky) were bubbled into 50 ml of 1:1 DMEM:Ham F12 medium using a vacuum pump apparatus. The CSE was filtered through a 0.22 μm filter to remove particles and bacteria before use. Solutions of 10% and 100% CSE were prepared from this stock. The solution of CSE (15 μl) was applied to the apical surface of the HBE cells, and RNA was isolated from the cultures at 2, 24, and 48 hr after CSE exposure using TRIzol (Life Technologies) followed by RNeasy (Qiagen). Samples were obtained in triplicate for each time point. Pirin RNA levels were measured by TAQMAN RT-PCT using the primers and probe described above. Pirin expression levels relative to 18s rRNA were assessed. Each data point was generated from triplicate wells for each of the three separate cell lines.

Assessment of Pirin-Induced Apoptosis. To assess the relationship between up-regulation of pirin and the induction of apoptosis, an adenovirus (Ad) gene transfer vector coding for pirin was used to transfer the human pirin cDNA to human bronchial epithelial cells, and pirin expression and apoptosis were assessed over time. The recombinant Ad vectors AdPirin and AdNull used in this study are E1a-, partial E1b-, and partial E3-, based on the Ad5 genome, with the expression cassette in the E1 position (Hersh, Gene Ther., 2: 124-131 (1995); Rosenfeld, Science, 252: 431-434 (1991); He, Proc. Natl. Acad. Sci. U.S.A., 95: 2509-2514 (1998)). The AdPirin expression cassette includes the cytomegalovirus early/intermediate enhancer/promoter (CMV), an artificial splice signal, the human pirin cDNA (obtained from A549 cells), and an SV40 stop/poly (A) signal. The AdNull vector is identical to the AdPirin vector, except that it lacks a cDNA in expression cassette (Hersh, Gene Ther., 2: 124-131 (1995)). The vectors were propagated, purified, and stored at −70° C. (Rosenfeld, Science, 252: 431-434 (1991)).

AdPirin-induced apoptosis was assessed in the human airway epithelial BEAS-2B cell line (Ke, Differentiation, 38: 60-66 (1998)). BEAS-2B cells (ATCC, Rockville, Md.) were grown on lysine-coated coverslips in LHC-9 medium (Biosource International, Camarillo, Calif.) until they were 50 to 60% confluent. The cells were then infected with AdNull and AdPirin at varying concentrations (103 and 104 particle units (pu), respectively).

Two assays were used to assess apoptosis: TdT-mediated dUTP nick end labeling (TUNEL) assay and cytoplasmic nucleosome ELISA. For the TUNEL assay, cells were fixed to the cover slips using 4% paraformaldehyde and then permeabilized with 0.2% Triton X-100 in PBS. Cells were equilibrated with equilibration buffer, nucleotide mix, and rTdT enzyme (Promega, Madison, Wis.) for 60 min and then washed. DAPI nuclear counterstain was applied before cells were mounted onto slides and evaluated under a fluorescent microscope. The percentage of apoptotic cells per 10× field were manually counted in 10 fields per slide. For the cytoplasmic nucleosome ELISA assay (Cell Death Detection ELISA, Roche, Indianapolis, Ind.), BEAS-2B cells were lysed with lysis buffer, centrifuged 10 min at 200 g to pellet nuclei. The supernatant (20 μl) was added to the immunoreagent containing anti-histone biotin and anti-DNA horseradish peroxidase (HRP). Sample wells were placed on shaker at 300 rpm for 2 hr, 23° C. 2,2-azino-di[3-ethylbenzthiazolin-sulfonate] (ABTS) solution was added, and photometric analysis was measured at 405 nm, subtracted from background 490 nm. For each sample, the fluorescent value was normalized to the internal negative control of the experiment to generate an apoptotic index, which reflects the fold change in the number of apoptotic cells for experimental condition compared to control.

Northern Analysis. A P-labeled pirin specific DNA probe (Spira, Proc. Natl. Acad. Sci. U.S.A., 101: 10143-10148 (2004)) was synthesized using strip EZ labeling kit (Roche, Indianapolis, Ind.) from a pirin cDNA template amplified from human genomic DNA. RNA electrophoresis was performed in 1% agarose gel followed by transfer to nitrocellulose membrane and UV crosslinking. The membrane was then hybridized with DNA probe and exposed to X ray film for 1 hr.

Statistical Analysis. The microarray data was analyzed using the GENESPRING software (Silicon Genetics, Redwood City, Calif.). Normalization was carried out sequentially: per microarray sample (dividing the raw data by the 50th percentile for all measurements) and then per gene (dividing the raw data by the median of the expression levels for the given gene in all samples). Data from probe sets representing genes that failed the Affymetrix detection criteria (labeled “Absent” or “Marginal”) in all 44 microarrays were eliminated from further analysis. The p value for each gene was calculated comparing the nonsmokers with smokers using the Welch t-test with a Benjamini-Hochberg correction for false discovery rate. For the in vitro studies, comparisons between RNA expression levels for pirin and percentage of apoptotic cells were made using the Student's two-tailed t-test.

Microarray Analysis. The microarray analysis was carried out in a data set previously reported using a total of 44 Affymetrix HuGene FL microarrays to assess left and right samples from 22 individuals, including 9 nonsmokers and 13 smokers (Hackett, Am. J. Respir. Cell. Mol. Biol., 29: 31-343 (2003); Kaplan, Cancer Res., 63: 1475-1482 (2003)). These 44 microarrays passed quality control as assessed by the GENESPRING software (Silicon Genetics, Redwood City, Calif.). The smokers and nonsmokers were comparable with respect to yield and percentage of non-epithelial cells. To eliminate genes not expressed in airway epithelium, or expressed at low levels, those genes that were called absent by the Microarray Suite software (Affymetrix) in all of the 44 microarrays were discarded before further analysis. The number of genes remaining (i.e., expressed on at least 1 of the 44 microarrays) was 4,512. Using this subset of genes, non-parametric statistical methods (GENESPRING software) were used to identify genes which were expressed at a higher or lower level in a significant number of smokers vs. nonsmokers. Of the 4,512 genes that were expressed, there were a total of 85 probesets that were significantly (p<0.05) up-regulated and 13 probesets down-regulated in smokers compared to nonsmokers. The 98 probesets were functionally annotated by manual review of public databases (e.g., Medline, Locuslink) into categories that described their cellular processes. Of these, 7 of the up-regulated genes were identified to be associated with apoptosis, including pirin, retinoic acid receptor responder 2, prostrate differentiation factor, insulin-like growth factor binding protein 5, bone morphogenic protein 7, carcinoembryonic antigen-related cell adhesion molecule 6, and 5100 calcium-binding protein A10 (Table 3). Of these, only two (retinoic acid receptor responder 2 and insulin-like growth factor binding protein 5) had any known association with cigarette smoke exposure. All of the genes identified were involved in signal transduction and transcription factors. Pirin, which has been shown to be induced during stress to cause cell death (Orazez, Plant Mol. Biol., 46: 459-468 (2001); Hihara, FEBS Lett., 574: 101-105 (2004)), was selected for further study because it had the highest fold change (3.12 smokers/nonsmokers) amongst genes in this category.

TABLE 3

| | | Smokers/non- | p |
|---|---|---|---|
| Gene ID¹ | Description | smokers (fold up) | value |
| Y07867 | pirin | 3.12 | 0.002 |
| U77594 | retinoic acid receptor responder 2 | 2.59 | 0.007 |
| AB000584 | prostate differentiation factor/ | 2.44 | 0.033 |
| | growth differentiation factor 15 | | |
| L27559 | insulin-like growth factor binding | 1.94 | 0.038 |
| | protein 5 | | |
| X51801 | bone morphogenetic protein 7 | 1.83 | 0.046 |
| | (osteogenic protein 1) | | |
| M18728 | carcinoembryonic antigen-related | 1.76 | 0.008 |
| | cell adhesion molecule 6 | | |
| M38591 | S100 calcium-binding protein A10 | 1.75 | 0.043 |

¹All up-regulated genes were evaluated for an association with apoptosis by reviewing published information about each gene available in public databases. Genes that had experimental evidence linking their expression to apoptosis were selected for further study.

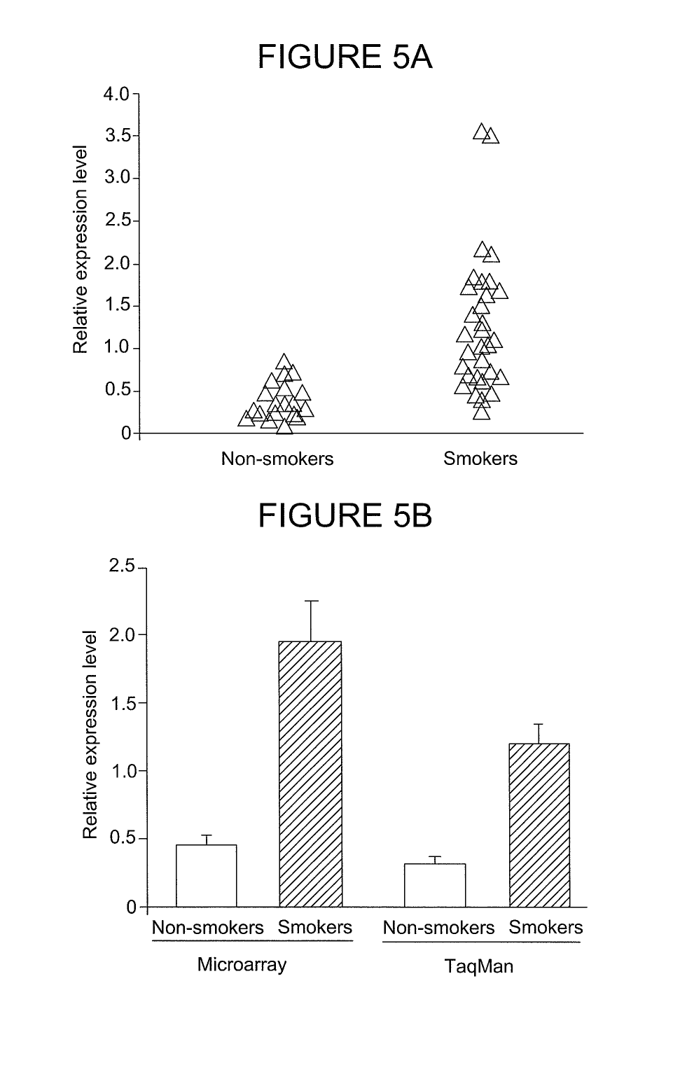

Up-regulation of Pirin in Cigarette Smoker's Bronchial Epithelium In Vivo. The microarray data demonstrated (n=18 samples from n=9 nonsmokers, n=26 samples from n=13 smokers) a significant up-regulation of pirin in the airway epithelium in the smokers (p=0.002; Table 3, FIG. 5A). To confirm the microarray data showing overexpression of pirin in the airway epithelium of smokers, pirin expression levels were assessed by an independent method using a subset (n=6 samples from n=3 nonsmokers, n=18 samples from n=9 smokers) of the RNA samples studied by microarray analysis, for which there was adequate amount of RNA available. TAQMAN confirmed that expression levels were significantly different between the two groups, reinforcing the validity of the observation with the microarray analysis that pirin mRNA levels are markedly elevated in the airway epithelium of smokers compared to nonsmokers (p<0.01; FIG. 5B).

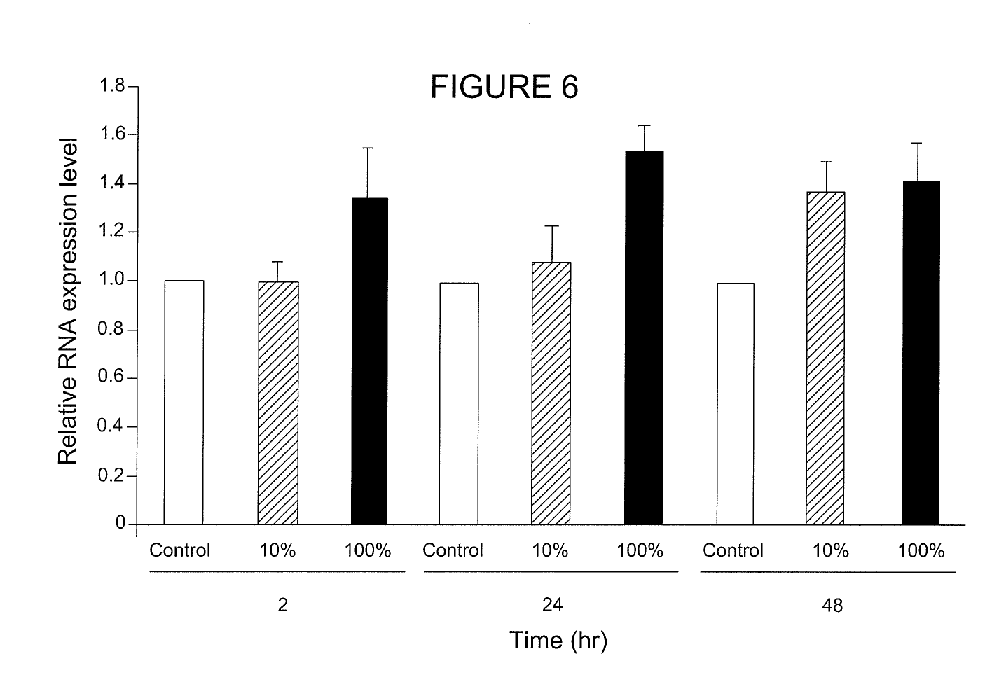

Pirin Gene Expression Following Cigarette Smoke Exposure In Vitro. To further establish that cigarette smoke up-regulates pirin expression in human bronchial epithelium, primary human bronchial epithelial cells were exposed to cigarette smoke extract in vitro. Human bronchial epithelial cells were used, as they most closely mimic airway epithelial cells in their natural environment in vivo (Karp, Methods Mol. Biol., 188: 115-137 (2002)). TAQMAN PCR with pirin RNA specific primers was used to quantify the amount of mRNA produced in the cells. Forty-eight hours after exposure to either 10% or 100% CSE, there was a respective 1.4-fold increase in pirin RNA levels in the cells exposed to cigarette smoke extract compared to the control group cultured in media (p<0.03, 10% and 100% compared to controls; FIG. 6).

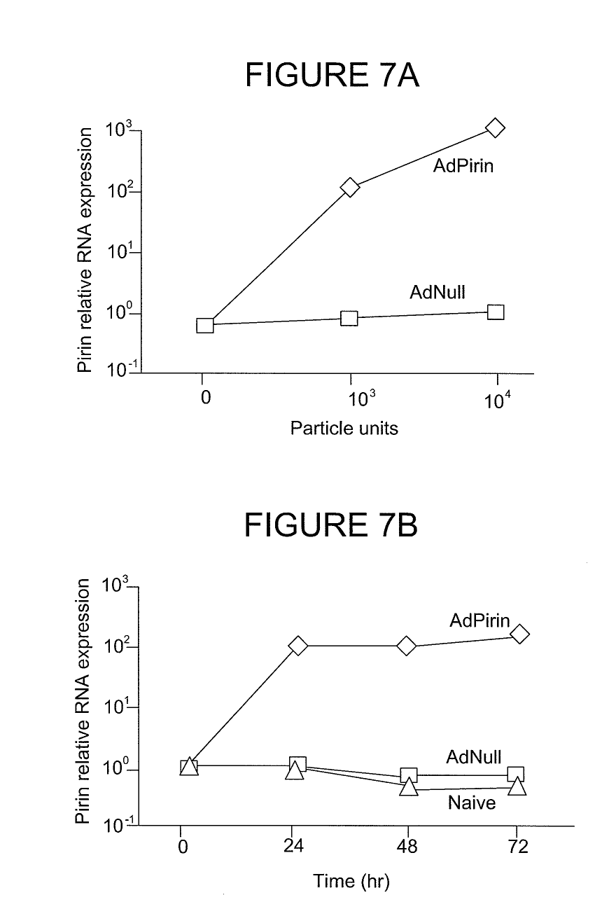

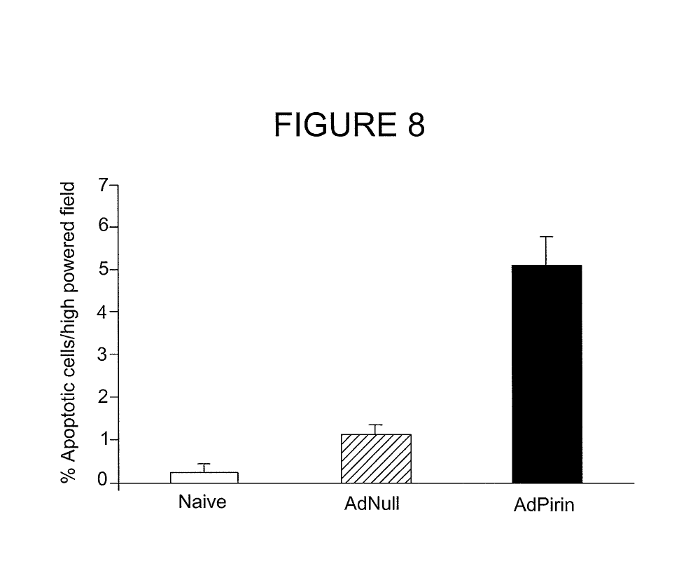

Induction of Bronchial Epithelial Cell Apoptosis in Association with Up-regulation of Pirin Expression. To test the hypothesis that up-regulation of pirin expression is linked to increased apoptosis in epithelial cells, an adenovirus vector expressing human pirin cDNA was used to modify the human bronchial epithelial BEAS-2B cell line to express high levels of pirin RNA. Northern analysis demonstrated AdPirin-specific up-regulation of the 1.1 kb pirin mRNA TAQMAN assessment of the pirin mRNA levels showed 79- and 538-fold change in expression at 103 and 104 particle units of AdPirin per cell, respectively (p<0.01; FIG. 7A). The expression of pirin was time-independent, with expression up-regulated>100-fold for AdPirin at 24 to 72 hr (FIG. 7B). Using Tat-mediated dUTP nick end labeling (TUNEL) to assess apoptosis, there was an approximately 5-fold increase in the number of TUNEL positive BEAS-2B cells exposed to 104 AdPirin compared to cells exposed to AdNull for 24 hr (p<0.01; FIGS. 7A-B, FIG. 8).

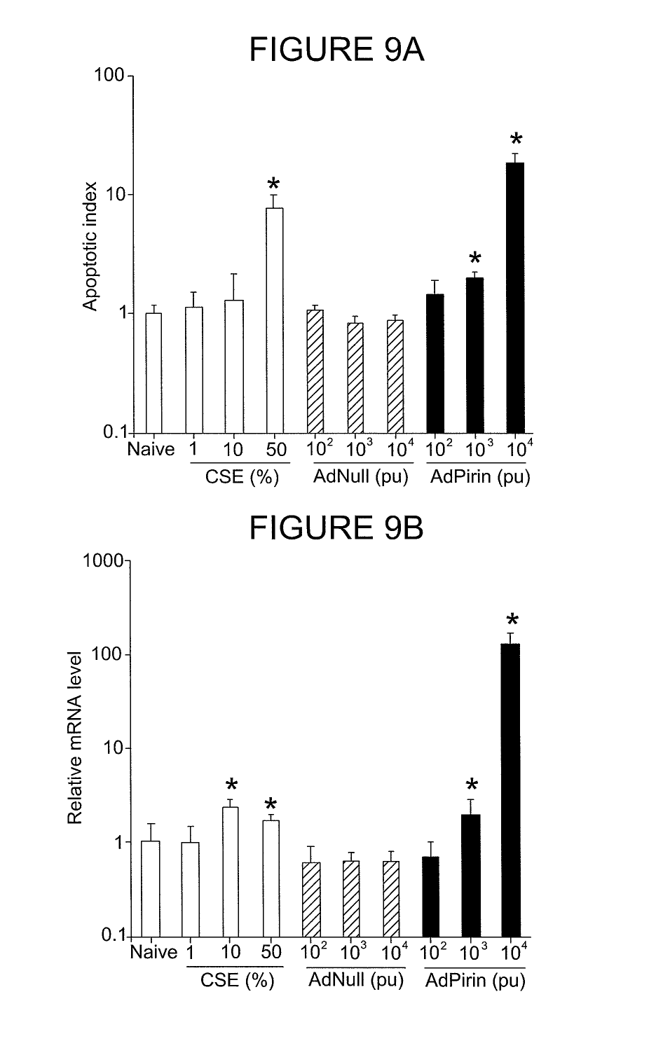

Confirmation of the TUNEL assay results were made using an ELISA against cytoplasmic nucleosomes. BEAS-2B cells were exposed to varying concentrations of AdPirin, AdNull, and cigarette smoke extract (CSE) and evaluated for apoptosis and pirin RNA level. This assay, which uses an increase in fluorescent signal compared to the naïve negative control to generate an apoptotic index, demonstrated a 19.3-fold increase for cells exposed to 104 AdPirin (p<0.01), a 2.1-fold increase for cells exposed to 103 AdPirin (p<0.01) and a 7.9 fold increase for cells exposed to 50% CSE (p<0.01, FIG. 9A). There was no significant increase in apoptotic cells compared to the naïve negative control for all other conditions. In this experiment, pirin RNA levels were increased 2.3-fold (p<0.01) and 1.7-fold (p<0.03) for BEAS-2B cells exposed to 10% and 50% CSE, respectively. BEAS-2B cells exposed to 103 and 104 AdPirin demonstrated a 2.0-fold (p<0.04) and 133.7-fold (p<0.01) increase in pirin RNA expression level, respectively (FIG. 9B).

### Example 16

This example compares the expression of genes in the lungs of smokers versus nonsmokers.

Study subjects. This study was approved by the Weill Cornell Medical College Institutional Review Board. Written informed consent was obtained from each individual prior to enrollment in the study. Individuals underwent an initial screening evaluation including history (detailed smoking habits), complete physical exam, blood studies, urine analysis, chest roentgenogram, lung function tests, and electrocardiogram (EKG). Special screening evaluation relevant to smoking habits included the urinary levels of nicotine and its derivative cotinine, and serum levels of carboxyhemoglobin. Upon completion of the baseline evaluation, those individuals who met the inclusion criteria (five nonsmokers and five smokers) underwent fiber-optic bronchoscopy and bronchoalveolar lavage (BAL) to obtain AM.

Collection of alveolar macrophages. Fiber-optic bronchoscopy was performed to obtain cells present in the BAL fluid (Russi, The Lung: Scientific Foundations, 2nd ed., Lippincott-Raven Publishers, Philadelphia, pp. 371-382 (1997)). The total volume used per site was typically 100 ml. A maximum of three sites were evaluated, with a total volume not exceeding 300 ml. Recovery of the infused volume was typically 45-65%. The right middle lobe, right lower lobe, and lingula were the usual sites for lavage. BAL fluid was filtered with gauze and centrifuged at 1,200 rpm for 5 min at 4° C. Cells were washed twice in RPMI 1640 containing 10% fetal bovine serum, 50 U/ml penicillin, 50 U/ml streptomycin, and 2 mM glutamine (Invitrogen, Carlsbad, Calif.), suspended in 10 ml medium, and an aliquot of 0.5 ml was used for a differential cell count. Cell viability was estimated by Trypan blue exclusion and expressed as a percentage of the total cells recovered. Total cell number was determined by counting on a hemocytometer. Differential cell count was assessed on sedimented cells prepared by cytocentrifugation (Cytospin 3; Shandon Instruments, Pittsburgh, Pa.) stained with DiffQuik (Baxter Healthcare, Miami, Fla.). The remainder was processed for RNA extraction by seeding the cells in six-well plastic culture dishes (2×106 per 2 ml/well) and purifying the AM by 2 hr adherence at 37° C. in a 5% CO₂ humidified incubator, removing the nonadherent cells by washing with RPMI 1640.

RNA extraction and preparation for Affymetrix microarrays. Total RNA was extracted using the TRIzol (Life Technologies, Gaithersburg, Md.) method followed by RNeasy clean-up (Qiagen, Valencia, Calif.) to remove residual DNA, a procedure giving a yield of 2 to 4 μg from 106 cells. Complementary DNA (cDNA) and complementary RNA (cRNA) synthesis was prepared and hybridized to the Affymetrix GeneChip HuGeneFL microarray, which enables the relative monitoring of messenger RNA (mRNA) transcripts of approximately 5,600 full-length human genes (6,800 probes), initially released by Affymetrix in November of 1998. All procedures were carried out as specified by Affymetrix (Santa Clara, Calif.).

Microarray data analysis. The data on each individual microarray chip were scaled to an arbitrary target intensity, as recommended by Affymetrix, using the Microarray Suite version 5.0 software. Normalization was carried out using the GENESPRING software (Agilent Biotechnologies, Palo Alto, Calif.) as follows: (1) per microarray sample, dividing the raw data by the 50th percentile of all measurements, and (2) per gene, by dividing the raw data by the median of the expression level for the gene in all samples. To eliminate those genes not expressed in the AM, only the genes with detectable expression in at least one out of the ten samples (Affymetrix Detection Call of Present in at least one of the ten samples) were chosen for further analysis. The statistical analysis was carried out for these 4,199 genes. Fold-changes were calculated as the ratio of the average expression level in the smokers to the average expression level in the nonsmokers. Clustering and tree building programs were used to compare the overall gene expression patterns among samples from smokers and nonsmokers for global comparisons of all 4,199 genes flagged as Present in at least one sample, as well as evaluations of the genes that were found to be differentially expressed in the smokers compared to the nonsmokers (see Statistics below). Normalized, log-transformed gene expression levels were evaluated using the Cluster program (Eisen, Proc. Natl. Acad. Sci. USA, 95: 14863-14868 (1998)) and subjected to hierarchical complete linkage clustering by both individual and gene. The resulting cluster was visualized with the TreeView program (Eisen, Proc. Natl. Acad. Sci. USA, 95: 14863-14868 (1998)).

TAQMAN mRNA analysis. To confirm the results of the microarray analysis, TAQMAN real-time reverse transcriptase (RT) polymerase chain reaction (PCR) analysis was used as an independent method of measuring gene expression levels. Samples from all five nonsmokers and four of the five smokers were assessed for three genes representative of novel observations [osteopontin, a disintegrin and metalloprotease domain 10 (ADAM 10), and chemokine (C-X-C motif) ligand 6]. First strand cDNA was synthesized from 2 μg of RNA in a 100 μl reaction volume, using the TAQMAN Reverse Transcriptase Reaction Kit (Applied Biosystems, Foster City, Calif.), with random hexamers as primers, and diluted with Universal Master Mix (Applied Biosystems) to 1:100 or 1:10. The probe and primers specific for mRNA were designed for each gene using the PrimerExpress software (Applied Biosystems). Each dilution was assayed in triplicate wells. Relative expression levels were calculated using the ΔΔCt method (Applied Biosystems), with ribosomal RNA (rRNA) as the internal control (Human Ribosomal RNA Kit, Applied Biosystems), and a cocktail consisting of equal parts of mRNA samples from the AM of the nonsmokers in this study, as the calibrator. The rRNA probe was labeled with VIC, and the probe for each of the three specific genes was labeled with FAM. The PCR reactions were run in an Applied Biosystems Sequence Detection System 7700. The relative quantity was calculated using the algorithm provided by Applied Biosystems.

Statistics. Comparison of the age of the subjects, cell yield and viability, and % cell types in the smokers and nonsmokers was performed by a two-tailed Student's t test. The significance of gene expression differences between the two groups was determined by calculating the p value for expression levels between the nonsmoker group and the smoker group using the Student's t test, assuming a two-tailed distribution and equal variances, with the log of the signal to background ratio as the starting value, using the GENESPRING software. To compare the results obtained using microarrays to those obtained using TAQMAN realtime RT-PCR, a two-way analysis of variance (ANOVA) was performed, using method (microarray vs. TAQMAN) and smoking status (smokers vs nonsmokers) as independent factors. For the ANOVA, expression levels were normalized separately for the microarray and TAQMAN analysis by dividing individual values by the average expression level of all nonsmokers and smokers for that method, to allow direct comparisons of values between the two methods.

Study population and alveolar macrophage samples. The study population included ten individuals (all men; five healthy nonsmokers and five phenotypically normal current smokers). The smokers had an average smoking history of 19±3 pack-years. The two groups were similar in regard to age (p>0.1, smokers' average 33±7 years; nonsmokers' average 37±6 years). All individuals were classified as normal based on standard medical history, physical exam, routine blood and urine tests, chest roentgenogram, EKG, and pulmonary function tests. Urine nicotine, urine cotinine, and venous carboxy-hemoglobin levels verified that the individuals who gave a history of current smoking were smokers and that those reporting nonsmoking were nonsmokers. Approximately three times as many macrophages were recovered from the BAL samples of smokers as compared to those recovered from the BAL samples nonsmokers (p<0.003). The purity and viability of the samples obtained from the nonsmokers was comparable to the purity of the samples obtained from smokers (p>0.6), and neither group had significant numbers (1%) of polymor-phonuclear cells contaminating the preparations.

Global gene expression patterns in the alveolar macrophages of healthy smokers and nonsmokers. Global gene expression patterns, i.e., the overall expression of 4,199 genes expressed in these samples, did not distinguish AM from smokers from those of nonsmokers. Clustering and tree building programs (Eisen, Proc. Natl. Acad. Sci. USA, 95: 14863-14868 (1998)) were used to compare the overall gene expression pattern among all samples from smokers and nonsmokers, using these 4,199 genes. The data show no overall clustering of global gene expression patterns in smokers as compared to nonsmokers.

Representative individual-to-individual comparisons (smoker to smoker, nonsmoker to nonsmoker, and smoker to nonsmoker) of expression levels (normalized by array only) of the 1,582 genes present on all ten microarrays indicated overall similarity in global gene expression levels among individuals, because all comparisons (smokers to smokers, nonsmokers to nonsmokers, and smokers to nonsmokers) revealed highly significant correlations with high r2 values (p<0.001 in all cases). Together, these observations indicate that the global changes in AM gene expression due to smoking in healthy individuals are modest relative to the overall biological variability among healthy human individuals.

Smoking alters the expression levels of specific genes in the alveolar macrophages of healthy smokers compared to nonsmokers. Assessment of the effect of smoking on the expression level of the 4,199 genes expressed in AM demonstrated that there were 40 up-regulated genes with p<0.05, and expression levels at least two-fold greater in smokers than in nonsmokers, and 35 down-regulated genes with p<0.05, and expression levels at least two-fold lower in smokers than in nonsmokers (Tables 4 and 5).

TABLE 4

| | | | | Smokers/nonsmokers | | |
|---|---|---|---|---|---|---|
| | Gene | | | Fold up- | Fold down- | |
| Category | ID¹ | Description | Gene symbol | regulated | regulated | p value² |
| Immune | HG4069- | Chemokine (C-C | CCL2⁴ | 4.9 | | 0.002 |
| response and | HT4339 | motif) ligand 2 | | | | |
| inflammation | | (MCP-1)³ | | | | |
| | HG1723- | Macrophase | MSR1⁴ | 3.6 | | 0.025 |
| | HT1729 | scavenger receptor | | | | |
| | | (scavenger receptor | | | | |
| | | type A) | | | | |
| | M98399 | CD36 antigen | CD36⁴ | 2.4 | | 0.043 |
| | HG1155- | Colony-stimulating | CSF1⁴ | 2.3 | | 0.041 |
| | HT4822 | factor 1 | | | | |
| | X99133 | Lipocalin 2 | LCN2⁵ | | 7.2 | 0.042 |
| Proteolysis/ | L23808 | Matrix | MMP12⁴ | 3.5 | | 0.038 |
| antiproteolysis | | metalloproteinase | | | | |
| | | 12 (macrophase | | | | |
| | | elastase | | | | |
| | M11313 | α2-macroglobulin | A2M⁴ | 3.2 | | 0.036 |
| Antioxidant- | X15722 | Glutathione | GSR⁵ | 3.2 | | 0.047 |
| related | | reductase | | | | |
| | X55448 | Glucose-6- | G6PD⁵ | 2.1 | | 0.025 |
| | | phosphate | | | | |
| | | dehydrogenase | | | | |
| | U30255 | Phosphogluconate | PGD⁵ | 2.1 | | 0.011 |
| | | dehydrogenase | | | | |
| Regulation of | M84820 | β-retinoid X | RXRB⁵ | 2.2 | | 0.037 |
| transcription | | receptor | | | | |
| Extracellular | Z26653 | α2-laminin | LAMA2⁵ | | 3.9 | 0.031 |
| matrix | | | | | | |
| Other | L26336 | 70 kDa heat shock | HSPA2⁵ | | 3.8 | 0.045 |
| | | protein 2 | | | | |

¹The Gene ID numbers starting with a letter followed by five numbers are GenBank accession numbers, those starting with the letters HG are The Institute for Genomic Research (TIGR) identifiers.
²Expression levels in alveolar macrophages of five nonsmokers and five smokers were compared using two-tailed Student's t test assuming variances.
³MCP-1 monocyte chemoattractant protein 1.
⁴Observed in prior studies in alveolar macrophages.
⁵Observed in prior studies of lung cells, tissue, or fluids other than alveolar macrophages.

TABLE 5

| | | | | Smokers/nonsmokers | | |
|---|---|---|---|---|---|---|
| | Gene | | | Fold up- | Fold down- | |
| Category | ID¹ | Description | Gene symbol | regulated | regulated | p value² |
| Immune | U20758 | Osteopontin (secreted | SPP1 | 5.5 | | 0.001 |
| response and | | phosphoprotein 1) | | | | |
| inflammation | U95626 | Chemokine (C-C motif) | CCR5 | 4.8 | | 0.006 |
| | | receptor 5 | | | | |
| | D83920 | Ficolin 1 | FCN1 | 2.7 | | 0.032 |
| | U18259 | Major histocompatibility | MHC2TA | 2.1 | | 0.047 |
| | | complex class II transactivator | | | | |
| | U83303 | Chemokine (C-X-C motif) | CXCL6 | | 5.2 | 0.001 |
| | | ligand 6 (granulocyte | | | | |
| | | chemotactic protein 2) | | | | |
| | L05512 | Histatin 1 | HTN1 | | 3.0 | 0.022 |
| | M30818 | Myxovirus resistance 2 | MX2 | | 2.3 | 0.033 |
| | X57351 | Interferon induced | IFITM3 | | 2.0 | 0.025 |
| | | transmembrane protein 3 | | | | |
| | M14058 | Complement component 1, r | C1R | | 2.0 | 0.028 |
| | | subcomponent | | | | |
| Adhesion and | L25851 | αE-integrin | ITGAE | 2.4 | | 0.008 |
| extracellular | X15882 | α2-collagen, type VI | COL6A2 | 2.3 | | 0.015 |
| matrix | L38608 | Activated leukocyte cell | ALCAM | 2.1 | | 0.010 |
| | | adhesion molecule | | | | |
| | M33308 | Vinculin | VCL | 2.0 | | 0.020 |
| | X69819 | Intercellular adhesion molecule | ICAM3 | | 2.4 | 0.045 |
| | | 3 | | | | |
| Signal | Y09561 | Purinergic receptor P2X | P2RX7 | 3.4 | | 0.036 |
| transduction | | (ligand-gated ion channel 7) | | | | |
| | HG1996- | Rap2 | RAP2A | 2.8 | | 0.004 |
| | HT2044 | | | | | |
| | D50640 | cyclic guanosine | PDE3B | 2.7 | | 0.005 |
| | | monophosphate-inhibited | | | | |
| | | phosphodiesterase 3B | | | | |
| | L07597 | 90 kDa polypeptide 1 of | RPS6KA1 | 2.1 | | 0.003 |
| | | ribosomal protein S6 kinase | | | | |
| | HG511- | mitogen-activated protein | MAPKAP1 | 2.1 | | 0.004 |
| | HT511 | kinase associated protein 1 | | | | |
| | L24564 | Ras-related associated with | RRAD | | 22.3 | 0.043 |
| | | diabetes | | | | |
| | X06182 | V-kit Hardy-Zuckerman 4 | KIT | | 3.3 | 0.026 |
| | | feline sarcoma viral oncogene | | | | |
| | | homolog | | | | |
| | D86969 | PHD finger protein 16 | PHF16 | | 2.6 | 0.040 |
| | M64572 | Nonreceptor type 3 protein | PTPN3 | | 2.4 | 0.028 |
| | | tyrosine phosphatase | | | | |
| Proteolysis/ | Z48579 | Disintegrin and | ADAM10 | 2.3 | | 0.029 |
| antiproteolysis | | metalloproteinase domain 10 | | | | |
| | M21188 | Insulin-degrading enzyme | IDE | 2.1 | | 0.018 |
| | U04313 | Serine (or cysteine) proteinase | SERPINB5 | | 18.3 | 0.016 |
| | | inhibitor 5 | | | | |
| Lysomal | Z31690 | Lipase A | LIPA | 3.4 | | 0.015 |
| function | J03263 | Lysosomal-associated | LAMP1 | 3.1 | | 0.036 |
| | | membrane protein 1 | | | | |
| | M29877 | α-L fucosidase 1 | FUCA1 | 2.4 | | 0.046 |
| Regulation of | L01042 | TATA element modulatory | TMF1 | 2.4 | | 0.048 |
| transcription | | factor 1 | | | | |
| | X59841 | Pre-B cell leukemia | PBX3 | 2.0 | | 0.012 |
| | | transcription factor 3 | | | | |
| | L19314 | Hairy and enhancer of split 1 | HES1 | | 10.0 | 0.027 |
| | U44754 | 43 kDa polypeptide 1 of the | SNAPC1 | | 3.0 | 0.049 |
| | | small nuclear RNA activating | | | | |
| | | complex | | | | |
| | U09413 | Zinc finger protein 135 | ZNF135 | | 2.4 | 0.040 |
| Antioxidant- | U62389 | Soluble [nicotinamide adenine | IDH1 | 2.5 | | 0.025 |
| related | | denucleotide phosphate | | | | |
| | | (oxidized form)] isocitrate | | | | |
| | | dehydrogenase 1 | | | | |
| Other | L27943 | Cytidine deaminase | CDA | 4.4 | | 0.001 |
| | L76465 | Hydroxyprostaglandin | HPGD | 3.3 | | 0.038 |
| | | dehydrogenase 15- | | | | |
| | | (nicotinamide adenine | | | | |
| | | dinucleotide) | | | | |
| | L10381 | Ribonuclease L | RNASEL | 3.1 | | 0.021 |
| | L31529 | β1-syntrophin | SNTB1 | 2.5 | | 0.045 |
| | U57623 | Fatty acid binding protein 3 | FABP3 | 2.9 | | 0.019 |
| | U66036 | Sulfotransferase family, 1C1 | SULT1C1 | 2.4 | | 0.020 |
| | U18009 | Vesicle amine transport protein | VAT1 | 2.4 | | 0.027 |
| | | 1 homolog (Torpedo | | | | |
| | | californica) | | | | |
| | Z67743 | Chloride channel 7 | CLCN7 | 2.2 | | 0.019 |
| | M74525 | Ubiquitin-conjugating enzyme | UBE2B | 2.2 | | 0.050 |
| | | E2B (RAD6 homolog) | | | | |
| | M18533 | Dystrophin | DMD | | 8.5 | 0.024 |
| | Z19574 | Keratin 17 | KRT17 | | 8.4 | 0.037 |
| | M13955 | Keratin 7 | KRT7 | | 4.6 | 0.017 |
| | HG3945- | Phospholipid transfer protein | PLTP | | 5.2 | 0.040 |
| | HT4215 | | | | | |
| | X01630 | Argininosuccinate synthetase | ASS | | 5.1 | 0.028 |
| | AB002366 | | KIAA0368 | | 5.0 | 0.026 |
| | S58544 | Sperm associated antigen 1 | SPAG1 | | 4.5 | 0.024 |
| | U68385 | Myeloid ecotropic viral | MEIS4 | | 4.4 | 0.030 |
| | | integration site 1 homolog 4 | | | | |
| | | (mouse) | | | | |
| | M19309 | Slow skeletal troponin T1 | TNNT1 | | 4.4 | 0.044 |
| | M64936 | Retinoic acid-inducible | HUMRIRT | | 3.8 | 0.026 |
| | | endogenous retroviral DNA | | | | |
| | U02081 | Neuroepithelial cell | NET1 | | 3.8 | 0.028 |
| | | transforming gene 1 | | | | |
| | U23070 | Bone morphogenetic protein | BAMBI | | 2.8 | 0.001 |
| | | and activin membrane-bound | | | | |
| | | inhibitor homolog (Xenopus | | | | |
| | | laevis) | | | | |
| | M81883 | Glutamate decarboxylase 1 | GAD1 | | 2.6 | 0.013 |
| | U90716 | Coxsackie virus and adenovirus | CXADR | | 2.6 | 0.043 |
| | | receptor | | | | |
| | L38933 | GT198, complete open reading | HUMGT19A | | 2.4 | 0.017 |
| | | frame | | | | |
| | U60975 | Sortilin-related receptor L | SORL1 | | 2.4 | 0.000 |
| | | (DLR class, A repeats- | | | | |
| | | containing) | | | | |
| | D16350 | SA hypertension-associated | SAH | | 2.2 | 0.047 |
| | | homolog (rat) | | | | |
| | U21936 | Solute carrier family 15 | SLC15A1 | | 2.2 | 0.002 |
| | | (oligopeptide transporter), | | | | |
| | | member 1 | | | | |

¹The Gene ID numbers starting with a letter followed by five numbers are GenBank accession numbers, those starting with the letters HG are TIGR identifiers.
²Expression levels in alveolar macrophages of five nonsmokers and five smokers were compared using two-tailed Student's t test assuming equal variances.

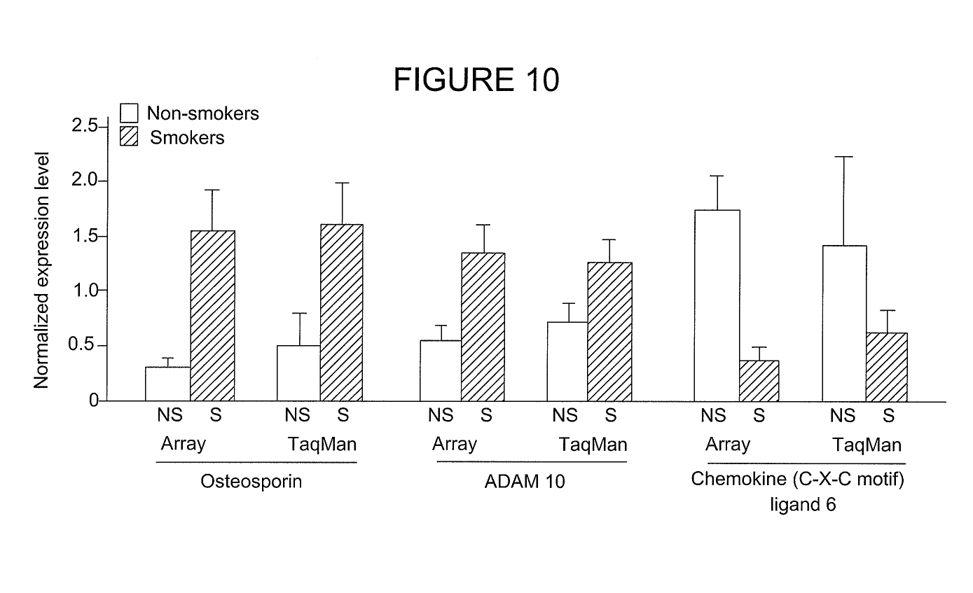

Confirmation of microarray results using TAQMAN real-time reverse transcriptase polymerase chain reaction. To confirm the results obtained with the microarray methodology, the expression levels of three of the differentially expressed genes that represent novel findings with respect to smoking were assessed by an independent method of RNA quantification, TAQMAN real-time RT-PCR, using the same RNA samples that were used for microarray analysis. Comparisons of relative expression levels for the two genes that were up-regulated in smokers (osteopontin and ADAM 10) and one gene that was down-regulated in smokers (chemokine (C-X-C motif) ligand 6) confirmed the validity of the microarray results for these genes (FIG. 10). These genes were selected for confirmation because they represented novel findings, but also because of their potential role in alveolar macrophage function. A two-way ANOVA confirmed that for each of the three genes there was a statistically significant effect of smoking (p<0.01 in all cases), but not methodology (p>0.7 in all cases), on expression levels. There was no significant effect of the interaction between smoking and methodology (microarray vs. TAQMAN) on expression levels of any of these three genes (p>0.4 in all cases).

Inter-individual variability in gene expression levels. Analysis of the pattern of expression by hierarchical clustering analysis of the 75 genes that were differentially expressed in smokers compared to nonsmokers suggested interindividual variability in expression levels within the two groups. For example, the serine protease member five of clade B of the serpin family (serpin B5; also known as maspin), and the gene encoding the ras-related gene associated with diabetes (RRAD) were markedly down-regulated in four of the five smokers, but expression levels of these genes in smoker one (S1) was similar to those found in nonsmokers. While S1 clustered with the other smokers, this individual's pattern of expression was also the most different from the other smokers, as attested by its assignment to its own branch by the clustering program. These data suggest that the levels of up- and down-regulation of gene expression in the AM of healthy smokers are variable among individual smokers, with subgroups of individuals showing similar patterns for specific groups of genes.

### Example 17

This example compares the expression of biomarkers in the lung in normal nonsmokers, normal smokers, smokers with early COPD, and smokers with established COPD.

Study population. Normal nonsmokers, healthy chronic smokers, smokers with early COPD, and smokers with established COPD were evaluated at the Weill Cornell NIH General Clinical Research Center under protocols approved by the Weill Cornell Medical College Institutional Review Board. Different arrays were used to assess the total of 114 samples from 81 individuals; the demographic data for each group and for each site (large and small airway epithelium) are presented in Table 6. Written informed consent was obtained from each individual before enrollment in the study. No individual in any study group had any variable that suggested evidence of a lung malignancy. Normal nonsmokers and normal smokers were determined to be phenotypically normal on the basis of clinical history and physical examination, routine blood screening tests, urinalysis, chest X-ray, electrocardiogram, and pulmonary function testing. Current smoking status was confirmed on history, venous carboxyhemoglobin levels, and urinalysis for nicotine levels and its derivative cotinine. Smokers were defined as having early COPD if they had a diffusion capacity for carbon monoxide (DLCO) of <80% predicted with no evidence of airflow obstruction on pulmonary function testing and/or high-resolution computed tomography scanning of the chest revealed evidence of emphysema. Smokers with established COPD were defined according to Global Initiative for Chronic Obstructive Lung Disease criteria. (Pauwels, Am. J Respir. Crit. Care Med, 163: 1256-76 (2001)).

TABLE 6

| | HuGeneFL array | | HG-U133A array | | | |
|---|---|---|---|---|---|---|
| | Large airways | | Large airways | | Small airways | |
| | Normal | Normal | Normal | Normal | Normal | Normal |
| Variable | nonsmokers | smokers | nonsmokers | smokers | nonsmokers | smokers |
| n | 9 | 13 | 5 | 6 | 5 | 6 |
| Sex (male/female) | 7/2 | 9/4 | 3/2 | 4/2 | 3/2 | 4/2 |
| Age (y) | 39 ± 13 | 38 ± 8 | 34 ± 5 | 39 ± 5 | 34 ± 5 | 39 ± 5 |
| Race (B/W/H) | 5/4/0 | 6/1/1 | 2/2/1 | 2/3/1 | 2/2/1 | 2/3/1 |
| Smoking history (pack- | 0 | 21 ± 9 | 0 | 24 ± 3 | 0 | 24 ± 3 |
| years) | | | | | | |
| Urine nicotine (ng/mL) | 8 ± 7 | 3,493 ± 1,110 | 2 ± 5 | 585 ± 609 | 2 ± 5 | 585 ± 609 |
| Urine cotinine (ng/mL) | Negative | 1,118 ± 457 | 34 ± 15 | 1,129 ± 460 | 34 ± 15 | 1,129 ± 460 |
| Venous CO-Hb | ND | 4.0 ± 2.0 | 0.5 ± 0.2 | 4.6 ± 1.8 | 0.5 ± 0.2 | 4.6 ± 1.8 |
| Pulmonary function | | | | | | |
| variables | | | | | | |
| FVC | 102 ± 3 | 102 ± 3 | 111 ± 10 | 109 ± 18 | 111 ± 10 | 109 ± 18 |
| FEV1 | 102 ± 2 | 102 ± 3 | 107 ± 12 | 100 ± 16 | 107 ± 12 | 100 ± 16 |
| FEV1/FVC | 101 ± 3 | 99 ± 2 | 95 ± 5 | 91 ± 4 | 95 ± 5 | 91 ± 4 |
| TLC | 103 ± 3 | 99 ± 3 | 100 ± 15 | 103 ± 14 | 100 ± 15 | 103 ± 14 |
| DLCO | 88 ± 2 | 85 ± 3 | 92 ± 11 | 91 ± 9 | 92 ± 11 | 91 ± 9 |
| Epithelial cells | | | | | | |
| Total number | 94. ± 3.5 | 8.5 ± 3.9 | 6.9 ± 1.6 | 8.6 ± 3.2 | 9.8 ± 5.8 | 7.0 ± 3.8 |
| recovered × 10⁶ | | | | | | |
| Percent | 99 ± 1 | 99 ± 1 | 98 ± 1 | 98 ± 1 | 96 ± 4 | 96 ± 4 |
| epithelial cells | | | | | | |
| Percent | 1 ± 1 | 1 ± 1 | 1 ± 1 | 1 ± 1 | 4 ± 3 | 4 ± 4 |
| inflammatory | | | | | | |
| Differential cell count | | | | | | |
| Ciliated | 44 ± 3 | 46 ± 4 | 50 ± 2 | 43 ± 3 | 80 ± 5 | 75 ± 6 |
| Secretory | 9 ± 2 | 8 ± 2 | 9 ± 4 | 10 ± 2 | 4 ± 1 | 4 ± 3 |
| Basal | 25 ± 4 | 22 ± 4 | 20 ± 3 | 27 ± 4 | 5 ± 3 | 8 ± 2 |
| Undifferentiated | 21 ± 6 | 24 ± 3 | 21 ± 4 | 20 ± 2 | 8 ± 2 | 9 ± 3 |

| | HG-U133 Plus 2.0 array | | | | | |
|---|---|---|---|---|---|---|
| | | | Small airways | | | |
| | | | | | | Estab- |
| | Large airways | | | | Early | lished |
| | Normal | Normal | Normal | Normal | COPD | COPD |
| Variable | nonsmokers | smokers | nonsmokers | smokers | smokers | smokers |
| n | 4 | 5 | 12 | 12 | 9 | 6 |
| Sex (male/female) | 4/0 | 2/3 | 10/2 | 7/3 | 6/3 | 5/1 |
| Age (y) | 36 ± 2 | 43 ± 2 | 42 ± 8 | 45 ± 4 | 51 ± 7 | 49 ± 6 |
| Race (B/W/H) | 2/2/0 | 3/2/0 | 6/4/2 | 5/5/0 | 5/3/1 | 2/4/0 |
| Smoking history (pack- | 0 | 24 ± 4 | 0 | 26 ± 9 | 35 ± 25 | 26 ± 13 |
| years) | | | | | | |
| Urine nicotine (ng/mL) | Negative | 254 ± 266 | Negative | 648 ± 265 | 485 ± 118 | 347 ± 155 |
| Urine cotinine (ng/mL) | Negative | 1,310 ± 716 | Negative | 1,263 ± 212 | 1,230 ± 435 | 835 ± 374 |
| Venous CO-Hb | 1.7 ± 0.9 | 3.9 ± 2.7 | 1.7 ± 0.7 | 3.3 ± 0.9 | ND | 1.4 ± 0.6 |
| Pulmonary function | | | | | | |
| variables | | | | | | |
| FVC | 107 ± 14 | 103 ± 11 | 105 ± 9 | 103 ± 12 | 98 ± 11 | 106 ± 11 |
| FEV1 | 104 ± 10 | 98 ± 10 | 105 ± 7 | 100 ± 14 | 89 ± 13 | 87 ± 25 |
| FEV1/FVC | 96 ± 5 | 94 ± 8 | 99 ± 7 | 97 ± 7 | 78 ± 4 | 66 ± 14 |
| TLC | 95 ± 9 | 94 ± 17 | 97 ± 8 | 96 ± 15 | 95 ± 13 | 107 ± 20 |
| DLCO | 111 ± 6 | 90 ± 7 | 95 ± 12 | 94 ± 10 | 60 ± 11 | 82 ± 19 |
| Epithelial cells | | | | | | |
| Total number | 6.5 ± 0.5 | 4.9 ± 1.0 | 5.3 ± 1.7 | 6.8 ± 2.2 | 7.3 ± 1.6 | 5.7 ± 0.5 |
| recovered × 10⁶ | | | | | | |
| Percent | 99 ± 1 | 99 ± 1 | 99 ± 1 | 99 ± 1 | 99 ± 1 | 96 ± 1 |
| epithelial cells | | | | | | |
| Percent | 1 ± 1 | 1 ± 1 | 1 ± 1 | 1 ± 1 | 1 ± 1 | 4 ± 1 |
| inflammatory | | | | | | |
| Differential cell count | | | | | | |
| Ciliated | 51 ± 4 | 42 ± 3 | 78 ± 7 | 75 ± 10 | 81 ± 3 | 71 ± 4 |
| Secretory | 8 ± 1 | 10 ± 2 | 7 ± 3 | 7 ± 3 | 4 ± 1 | 9 ± 4 |
| Basal | 23 ± 1 | 27 ± 2 | 7 ± 2 | 9 ± 4 | 7 ± 3 | 2 ± 1 |
| Undifferentiated | 17 ± 3 | 20 ± 3 | 8 ± 4 | 9 ± 4 | 7 ± 1 | 2 ± 1 |

Abbreviation: ND, not determined.

Sampling of airway epithelial cells. Epithelial cells from the large and small airways were sampled using fiberoptic bronchoscopy as previously described (Hackett, Am. J. Respir. Cell. Mol. Biol., 29:331-43 (2003); Harvey, Proc. Am. Thorac. Soc., (2006)). Smokers were asked not to smoke the evening before the procedure. After achieving mild sedation and anesthesia of the vocal cords, a fiberoptic bronchoscope (Pentax, EB-1530T3) was advanced to the desired bronchus. Large airway epithelial samples were collected by gentle brushing of the third- to fourth-order bronchi and small airway samples were collected from 10th- to 12th-order bronchi. These cells were subsequently collected in 5 mL of bronchial epithelium basal cell medium (Clonetics, Walkersville, Md.). An aliquot was used for cytology and differential cell count, and the remainder was processed immediately for RNA extraction. Total cell counts were obtained using a hemocytometer, whereas differential cell counts (epithelial versus inflammatory) were determined on sedimented cells prepared by centrifugation (Cytospin 11, Shandon Instruments, Pittsburgh, Pa.) and stained with DiffQuik (Baxter Healthcare, Miami, Fla.).

RNA extraction and microarray processing. Analyses were done using three different Affymetrix (Santa Clara, Calif.) microarrays, including the HuGeneFL array (7,000 probe sets), HG-U133A array (22,000 probe sets), and HG-U133 Plus 2.0 array (54,000 probe sets). The protocols used were as described by the manufacturer. Total RNA was extracted from epithelial cells using TRIzol (Invitrogen, Carlsbad, Calif.) followed by RNeasy (Qiagen, Valencia, Calif.) to remove residual DNA. This process yielded 2 to 4 μg RNA per 106 cells. Samples analyzed using the HuGeneFL and HG-133A microarrays were processed as previously described by Hackett (Am. J. Respir. Cell. Mol. Biol., 29: 331-43 (2003); Harvey, Proc. Am. Thorac. Soc., (2006)), using 6 μg RNA. For samples analyzed using the HG-U133 Plus 2.0 array, an aliquot of each RNA sample was run on an Agilent Bioanalyzer (Agilent Technologies, Palo Alto, Calif.) to visualize and quantify the degree of RNA integrity. The concentration was determined using a NanoDrop ND-1000 spectrophotometer (NanoDrop Technologies, Wilmington, Del.). Three quality control criteria were used for an RNA sample to be accepted for further processing: (a) A260/A28O ratio between 1.7 and 2.3, (b) concentration within the range of 0.2 to 6 μg/mL, and (c) Agilent electropherogram displaying two distinct peaks corresponding to the 28S and 18S rRNA bands at a ratio of 28S/18S of >0.5 with minimal or no degradation. Double-stranded cDNA was synthesized from of 3 μg total RNA using the GeneChip One-Cycle cDNA Synthesis kit, followed by cleanup with GeneChip Sample Cleanup Module, in vitro transcription (IVT) reaction using the GeneChip IVT Labeling kit, and clean-up and quantification of the biotin-labeled cRNA yield by spectrophotometric analysis. All kits were from Affymetrix. Hybridizations to test chips and to the microarrays were done according to Affymetrix protocols, and microarrays were processed by the Affymetrix fluidics station and scanned with an Affymetrix GeneArray 2500 (HuGeneFL) and the Affymetrix GeneChip Scanner 3000 7G (HG-U133A and HG-U133 Plus 2.0). To maintain quality, only samples hybridized to test chips with a 3′ to 5′ ratio of <3 were deemed satisfactory.

Microarray data analysis. Captured images were analyzed using Microarray Suite version 5.0 algorithm (Affymetrix). These data were normalized using GENESPRING version 6.2 software (Agilent Technologies) as follows: (a) per array, by dividing raw data by the 50th percentile of all measurements, and (b) per gene, by dividing the raw data by the median expression level for all the genes across all arrays in a data set. All HG-U133A data and HG-U133 Plus 2.0 large airway data was log transformed before statistical analysis. To evaluate neuroendocrine cell-specific gene expression in the large and small airway samples of nonsmokers, healthy smokers, smokers with early COPD, and smokers with established COPD, a list of known neuroendocrine cell-specific genes was established from the literature (Adriaensen, Anat. Rec., 236: 70-85 (2003); Gosney, Microsc. Res. Technol., 37: 107-13 (1997); Scheuermann, LippinCott-Raven Publishers, pp. 603-13 (1997); Cutz, Experientia, 37:765-7 (1981); Jiang, Mod. Pathol., 17: 222-9 (2004); Mbikay, Biochem. J., 357: 329-42 (2001)). This signature transcriptome of neuroendocrine cells was used to assess the effects of smoking on the genome of these cells. Expression was defined as having an Affymetrix Detection Call of Present (P call) in >50% of samples assessed by each type of microarray.

TAQMAN reverse transcription-PCR confirmation of microarray expression levels. TAQMAN real-time reverse transcription-PCR(RT-PCR) was done on available RNA samples from the small airways of 12 normal nonsmokers and 10 normal smokers that had been assessed with the HG-U133 Plus 2.0 array. cDNA was synthesized from 2 μg RNA in a 100 μL reaction volume using the TAQMAN Reverse Transcriptase Reaction kit (Applied Biosystems, Foster City, Calif.), with random hexamers as primers. Two dilutions of 1:50 and 1:100 were made from each sample, and triplicate wells were run for each dilution. TAQMAN PCR reactions were carried out using premade gene expression assays for neuroendocrine genes from Applied Biosystems, and 2 μL cDNA were used in each 25 μL reaction volume. The endogenous control was 18S rRNA, and relative expression levels were determined using the ΔΔCt method (Applied Biosystems) with the average value for the nonsmokers as the calibrator. The PCR reactions were run in an Applied Biosystems Sequence Detection System 7500.

Localization of UCHL 1 in the airway epithelium. To determine which airway epithelial cells express UCHL1, bronchial biopsies were obtained from the large airway epithelium of six nonsmokers and six normal smokers using conventional methods. Immunohistochemistry was subsequently done on paraffin-embedded endobronchial biopsies. Sections were depar-affinized and rehydrated through a series of xylenes and alcohol. To enhance staining, an antigen retrieval step was carried out by microwave treatment of the sections at 100° C. for 15 minutes in citrate buffer solution (Labvision, Fremont, Calif.) followed by cooling at 23° C. for 20 minutes. Endogenous peroxidase activity was quenched using 0.3% H₂0₂ and blocking with normal goat serum to reduce background staining. Samples were incubated with the primary antibody at 23° C. for 1 hour. For chromogranin A (CHGA), the primary antibody was mouse monoclonal (LK2H10+PHE5) anti-human antibody (Labvision) diluted 1:5,000, and mouse IgG1 was the isotype control. For UCHL1 detection, the primary antibody was rabbit polyclonal anti-human UCHL1 (Labvision) diluted 1:2,500, and rabbit IgG (DakoCytomation, Carpinteria Calif.) was the isotype control. To block UCHL1 antibody binding, the UCHL1 antibody was incubated with the full-length recombinant UCHL1 protein (Labvision) at 23° C. for 30 minutes to saturate binding sites before being applied to sample tissues. Vectastain Elite ABC kit (Vector Laboratories, Burlingame, Calif.) and 3,3′-diaminobenzidine substrate kit (Vector Laboratories) were used to visualize antibody binding. The sections were counterstained with hematoxylin (Sigma Aldrich, St. Louis, Mo.) and mounted using GVA mounting medium (Zymed, San Francisco, Calif.). Brightfield microscopy was done using a Nikon Microphot microscope equipped with a Plan X40 numerical aperture (NA) 0.70 objective lens. Images were captured with an Olympus DP70 CCD camera.

Immunofluorescent staining was carried out on airway epithelial biopsies using primary antibodies for UCHL1 and CHGA as described above, mouse monoclonal (ONS1A6) anti-human β IV tubulin (1/500 dilution; Biogenex, San Ramon, Calif.) as a marker for ciliated cells, (Caballero, Oncogene, 21: 3003-10 (2002)); mouse monoclonal (45M1) mucin 5AC (1/200; Labvision) as a marker for secretory cells (Zuhdi, Am. J. Respir. Cell. Biol., 22: 253-60 (2000)); and mouse monoclonal (SH-L1) S100 A2 (1/50 dilution; GeneTex, Inc. San Antonio, Tex.) as a marker for basal cells (Smith, Br. J. Cancer, 91: 1515-24 (2004)). Following incubation with the primary antibodies at 23° C. for 1 hour in a humidified chamber, goat anti-rabbit Cy5 conjugated AffiniPure F(ab′)2 (Jackson Immunoresearch, West Grove, Pa.) at 1/100 dilution was used as a secondary antibody for UCHL1, and goat anti-mouse Cy3-conjugated AffiniPure F(ab′)₂ (Jackson Immunoresearch) at 1/100 dilution was used as a secondary antibody for all other antibodies. Fluorescence microscopy was done using a Zeiss LSM 510 Laser Scanning Confocal microscope equipped with a Plan Neofluor X40 NA 0.75 objective lens. Illumination was provided by an argon laser (488 nm line) and two helium/neon lasers (543 and 633 nm lines) with matched dichroic mirrors and emission filters. Images were analyzed using Zeiss LSM Image Browser version 3.1.099. Pseudocolor images were formed by encoding Cy5 fluorescence in the green channel, Cy3 fluorescence in the red channel, and autofluorescence in gray scale. The images were composed by integrating five independent images collected at a step size of 1.7 μm.

Statistical analysis. For all HuGeneFL data and small airway data analyzed on the HG-U133 Plus 2.0, P values for all comparisons were calculated using a two-tailed t test, assuming unequal variance (Welch t test) with the Benjamini-Hochberg multiple test correction for false-discovery rate (Benjamini, J. R. Statist. Soc. B, 57: 289-300 (1994)), using the GENESPRING software. Genes were considered significant if the Benjamini-Hochberg corrected P value was <0.05. For HG-U133A large and small airway data and for large airway data analyzed on the HG-U133 Plus 2.0 microarray, P values were calculated as described above, but in the absence of the Benjamini-Hochberg correction. Average expression values for neuroendocrine cell-specific genes in large and small airway samples were calculated from normalized expression levels for nonsmokers, normal smokers, smokers with early COPD, and smokers with established COPD. TAQMAN data was normalized per gene by dividing by the median expression of each gene in all samples, and subsequently the mean and SE were calculated for normalized values of expression. P values for TAQMAN data were calculated using the Welch t test.

Study population. A total of 114 samples were assessed from 81 study individuals (Table 6). Results were obtained using three different microarrays including: (a) HuGeneFL microarray—18 large airway samples from 9 nonsmokers and 26 large airway samples from 13 normal smokers; (b) HG-U133A array—large and small airway samples from 5 nonsmokers and 6 normal smokers; and (3) HG-U133 Plus 2.0 array—large airway samples from 4 normal nonsmokers and 5 normal smokers, and small airway samples from 12 normal nonsmokers, 12 normal smokers, 9 smokers with early COPD, and 6 smokers with established COPD. All individuals had no significant prior medical history and normal physical examinations. There were no differences between groups with regard to sex, race, or age (P>0.05 for all comparisons). There was a statistically significant difference in age in the nonsmoker group versus early COPD group (P<0.01) analyzed with the HG-U133 Plus 2.0 microarray. All individuals were HIV negative with blood and urine variables within reference ranges (P>0.05 all comparisons). Smokers had an average smoking history of 27±2 pack-years. The number of cells recovered by brushing ranged from 4.9×10⁶ to 9.8×10⁶ (Table 6). In all cases, >95% of cells recovered were epithelial cells. The subtypes of airway epithelial cells were as expected from the large and small airways (Table 6; Pauwels, Am. J. Respir. Crit. Care Med., 163: 1256-76 (2001)). Neuroendocrine cells were not observed in brushed airway samples.

Detection of neuroendocrine gene expression in the large and small airway epithelium. With the criteria of P call of ≧50%, most neuroendocrine genes were not detected in the large airway epithelium of nonsmokers [secretory granule neuroendocrine peptide 1 (SGNE1), pro-enkephalin (PENK), tachykinin 1 (TAC1), achaete scute homalogue 1 (ASCL1), neuronal cell adhesion molecule 1 (NCAM1), calcitonin gene-related polypeptide β (3 (CALCB), CHGA, gastrin releasing peptide (GRP and UCHL1 (Table 7)]. Of the 11 neuorendocrine genes evaluated, only expression of enolase 2 (ENO₂) was universally detected in the large airways of nonsmokers. In the small airways of normal nonsmokers, 5 of the 11 neuroendocrine genes were not expressed (SGNE1, PENK, TAC1, ASCL1, CALCB, and UCHL1), one gene was equivocal (NCAM1 was detected in only the HG-U133 Plus 2.0 array), and four genes were clearly detected [secretogranin 2 (SCG2), CHGA, ENO₂, and GRP].

TABLE 7

| | % P call | | | | | | | | | |
|---|---|---|---|---|---|---|---|---|---|---|
| | HUGene FL chip | | Hu 133A chip | | | | HG-U133 Plus 2.0 chip | | | |
| | Large airways | | Large airways | | Small airways | | Large airways | | Small airways | |
| | Normal | Normal | Normal | Normal | Normal | Normal | Normal | Normal | Normal | Normal |
| Gene Symbol | nonsmokers | smokers | nonsmokers | smokers | nonsmokers | smokers | nonsmokers | smokers | nonsmokers | smokers |
| SGNEI | NA | NA | 20 | 0 | 17 | 50 | 0 | 0 | 17 | 58 |
| PENK | NA | NA | 40 | 17 | 20 | 17 | 25 | 40 | 42 | 25 |
| TAC1 | NA | NA | 0 | 17 | 20 | 0 | 0 | 0 | 8 | 0 |
| ASCL1 | 0 | 8 | NA | NA | NA | NA | 0 | 20 | 33 | 60 |
| NCAM1 | NA | NA | 0 | 33 | 20 | 0 | 25 | 40 | 67 | 33 |
| CALCB | 11 | 23 | 40 | 0 | 0 | 33 | 40 | 0 | 0 | 8 |
| SCG2 | 0 | 4 | 20 | 66 | 20 | 83 | 75 | 100 | 92 | 83 |
| CHGA | NA | NA | 0 | 0 | 0 | 17 | 25 | 80 | 58 | 75 |
| ENO2 | 67 | 62 | 80 | 66 | 100 | 100 | 100 | 60 | 100 | 92 |
| GRP | 22 | 85 | 0 | 17 | 20 | 33 | 0 | 60 | 67 | 92 |
| UCHL1 | 0 | 69 | 0 | 100 | 0 | 100 | 0 | 80 | 8 | 100 |

| | % P call | | | | | | | |
|---|---|---|---|---|---|---|---|---|
| | HG-U133 Plus 2.0 chip | | Overall assessment of expression* | | | | | |
| | Small airways | | | | Small airways | | | |
| | Early | Established | Large airways | | | | Early | Established |
| | COPD | COPD | Normal | Normal | Normal | Normal | COPD | COPD |
| Gene Symbol | smokers^{†} | smokers^{‡} | nonsmokers^{§} | smokers | nonsmokers | smokers | smokers | smokers |
| SGNEI | 56 | 17 | No | No | No | ±* | Yes | No |
| PENK | 33 | 17 | No | No | No | No | No | No |
| TAC1 | 0 | 0 | No | No | No | No | No | No |
| ASCL1 | 44 | 50 | No | No | No | ± | ± | Yes |
| NCAM1 | 56 | 33 | No | No | ± | Yes | No | No |
| CALCB | 0 | 0 | No | No | No | No | No | No |
| SCG2 | 89 | 83 | ± | Yes | Yes | Yes | Yes | Yes |
| CHGA | 100 | 100 | No | ± | Yes | Yes | Yes | Yes |
| ENO2 | 100 | 83 | Yes | Yes | Yes | Yes | Yes | Yes |
| GRP | 100 | 83 | No | Yes | Yes | Yes | Yes | Yes |
| UCHL1 | 89 | 100 | No | Yes | No | Yes | Yes | Yes |

Abbreviation: NA, probe not on array, not applicable.
*Overall assessment of expression was based on P call ≧50% in at least two of the three arrays used. ± means probably expressed, but observed in only one type of array; this may be dependent on different probes on the different arrays.
^{†}Early COPD smokers - smokers with normal lung function except for abnormal DLCO.
^{‡}Established COPD smokers - smokers with COPD as defined by the GOLD criteria (29).
^{§}“No” indicates not expressed with a P call <50% and “Yes” indicates expression with a P call ≧50% (bold type).

In the current smokers (phenotypically normal, early COPD, and established COPD), expression of the neuroendocrine-specific genes in the large and small airways was mostly consistent with that observed in the large and small airway epithelium in nonsmokers (Table 7). However, in marked contrast to the other neuroendocrine genes, whereas UCHL1 was not detected in any of the large and small airway epithelial samples of the nonsmokers, UCHL1 was detected in the large and small airway epithelium of smokers in almost every microarray (large airway epithelium samples—69% of normal smokers assessed with the HuGeneFL chip; 100% of normal smokers with HG-U133A, and 80% of normal smokers with HG-U133 Plus 2.0; small airway epithelium samples—100% of normal smokers assessed with HG-U133A, 100% of normal smokers with HG-U133 Plus 2.0, 89% early COPD smokers with HG-U133 Plus 2.0, and 100% of established COPD smokers with HG-U133 Plus 2.0).

UCHL1 expression was 18.3-fold higher in normal smokers compared with nonsmokers in the large airways analyzed with the HuGeneFL array (p<0.01), 9.0-fold higher in large airway analyzed with the HG-U133A array (p<0.01), and 42.2-fold higher in large airways analyzed with the HG-U133 Plus 2.0 array (p<0.01). In the small airways, UCHL1 was 11.4-fold higher in normal smokers than nonsmokers in the HG-U133A array (p<0.01). In the HG-U133 Plus 2.0 data set, UCHL1 expression was 39.3-fold higher in normal smokers (p<0.01), 60.8-fold higher in smokers with early COPD (p<0.01), and 38.6-fold higher in smokers with established COPD (p<0.01). There was no significant difference in the level of expression of UCHL1 between normal smokers and smokers with early COPD (p>0.8) or smokers with established COPD (p>0.9).

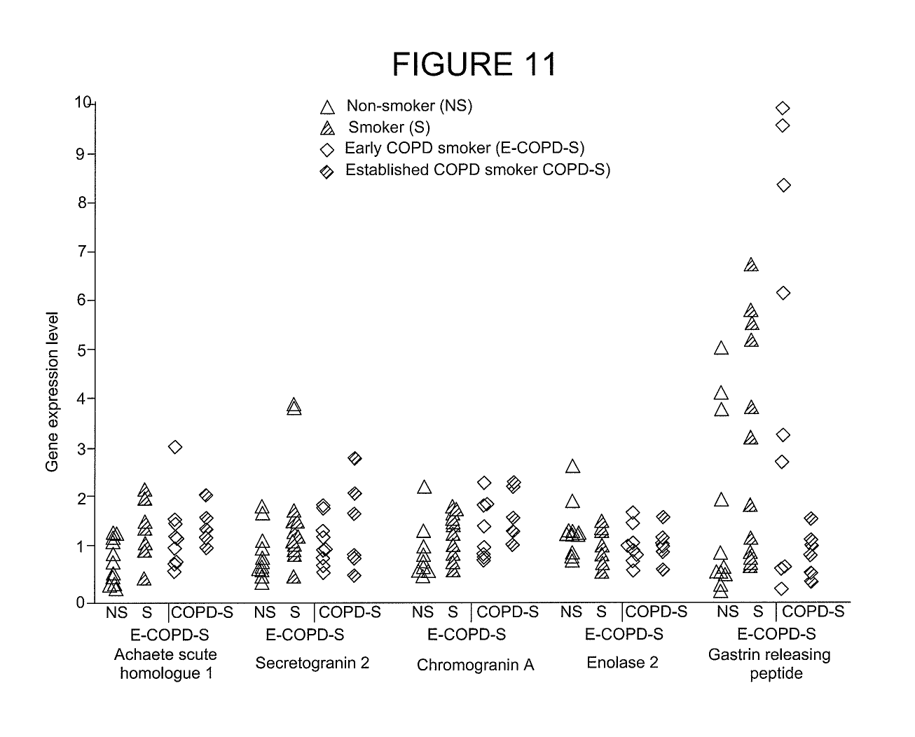

Quantitative expression of the neuroendocrine cell-specific genes in the small airway epithelium. Of the neuroendocrine-specific genes expressed in the airway epithelium, quantitative assessment of the relative gene expression levels showed no difference among nonsmokers and smokers for GRP, ENO₂, or SCG2 (FIG. 11; p>0.1 for all comparisons of nonsmokers to each of the current smoker groups including phenotypically normal smokers, smokers with early COPD, and smokers with established COPD). There was a significant difference in expression levels of CHGA in smokers with established COPD compared with normal nonsmokers (p<0.04).

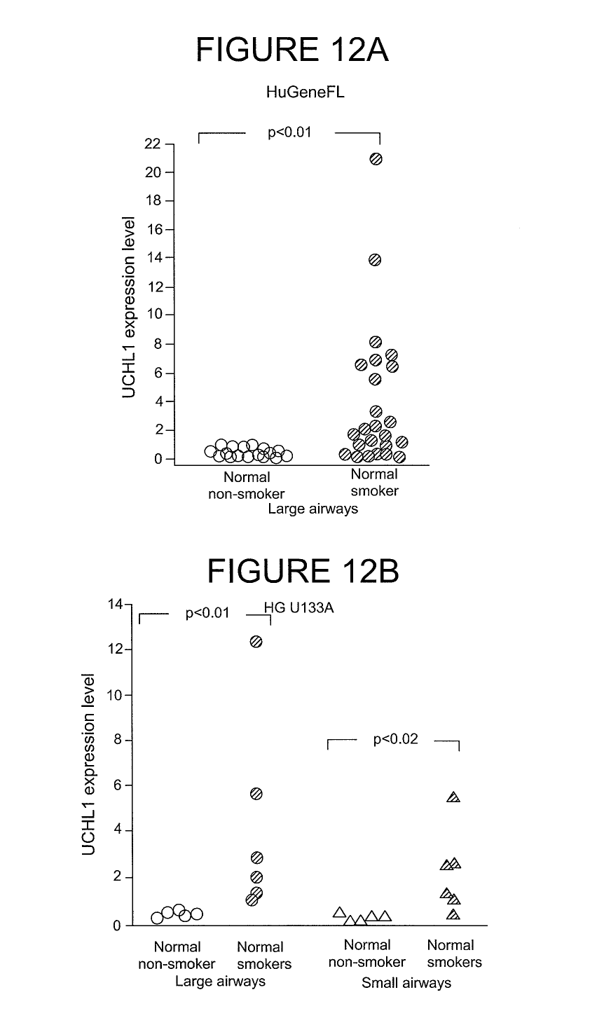

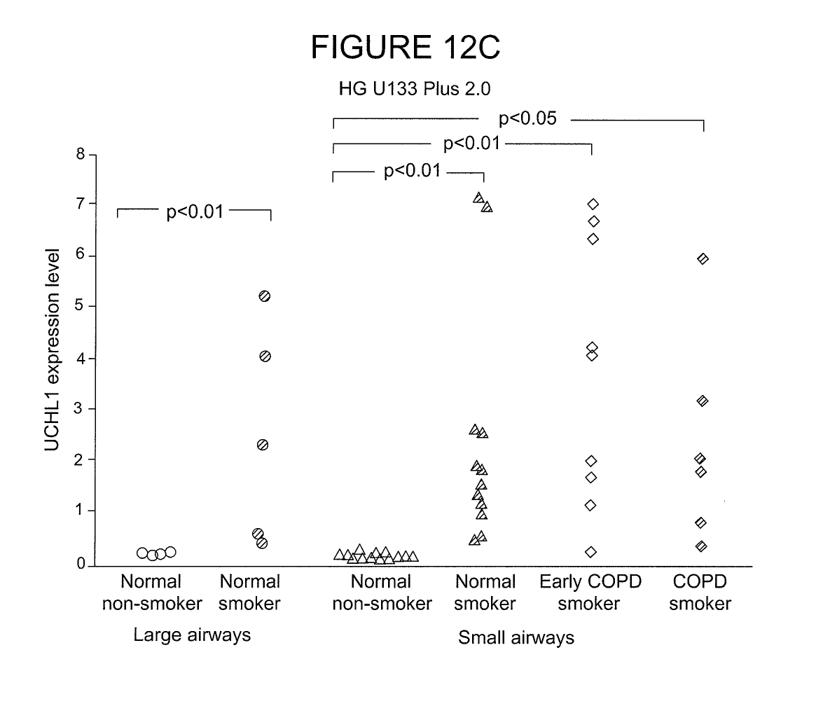

In marked contrast, the expression of UCHL1 was up-regulated in smokers compared with nonsmokers in the small and large airway epithelium in all the data sets (FIGS. 12A-C). This was true for normal smokers compared with normal nonsmokers in the large airway epithelial samples assessed with the HuGeneFL microarray (A; p<0.01), normal smokers compared with normal nonsmokers of the large airway epithelium assessed with the HG-U133A array (B; p<0.01); normal smokers compared with normal nonsmokers of the small airway epithelium assessed with the HG-U133A array (B; p<0.01), normal smokers compared with normal nonsmokers of the large airway epithelium assessed with the HG-U 133 Plus 2.0 array (C; p<0.01), and the normal smokers (p<0.01), early COPD smokers (p<0.01), and established COPD smokers (p<0.05) compared with normal nonsmokers of the small airway epithelium with the HG-U133 Plus 2.0 array (C).

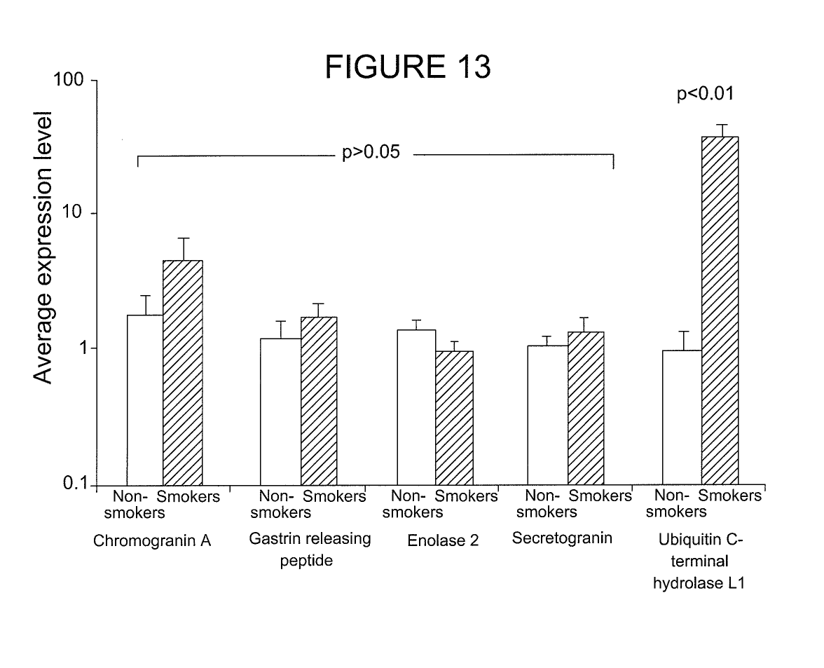

TAQMAN RT-PCR confirmation of microarray results. To confirm the results obtained from microarray studies, TAQMAN RT-PCR was carried out on RNA samples from the small airways of 12 normal nonsmokers and 10 normal smokers (FIG. 13). The TAQMAN data confirmed that there was no difference in expression levels of other neuroendocrine-specific genes, including CHGA, GRP, ENO2, and SCG2. The TAQMAN analysis also confirmed the up-regulation of UCHL1 mRNA expression in normal smokers compared with nonsmokers (p<0.01).

Localization of UCHL1 in the airway epithelium of smokers. Immunohistochemistry was used to assess expression of CHGA and UCHL1 in endobronchial biopsies obtained from large airways at bronchoscopy from six nonsmokers and six normal smokers. This analysis showed protein expression of CHGA and UCHL1 in airway epithelial cells with the typical morphology and localization of neuroendocrine cells. Surprisingly, the smokers not only had UCHL1 expression in typical neuroendocrine cells, but there was also positive staining for UCHL1 in other epithelial cells more apically in the airway epithelium that was not present in nonsmokers. To confirm the specificity of the polyclonal rabbit anti-UCHL1 antibody, a blocking step was done with full-length recombinant UCHL1 protein; this completely blocked all antibody binding on biopsy samples from smokers, thereby demonstrating the specificity of this polyclonal antibody for the UCHL1 epitope. Overall, although there were a greater number of cells with a neuroendocrine morphology observed in the airway epithelium of normal smokers compared with nonsmokers, there also were a greater number of UCHL1-positive cells within the airway epithelium of smokers compared with nonsmokers that were not positive for the neuroendocrine marker CHGA. These additional UCHL1-positive cells had the appearance and morphology of ciliated epithelial cells. UCHL1 was confirmed to be present in ciliated airway epithelial cells in the smokers as evidenced by colocalization with the ciliated cell-specific marker β IV tubulin but not with the secretory cell marker MUC5AC. The colocalization of UCHL1 and β IV tubulin was almost universal throughout the cilia with some cilia being more intensely positive for UCHL1, whereas, as expected, all cilia stained positive for β IV tubulin. UCHL1 was not present in basal cells as evidenced by lack of colocalization with S100 A2, which is a marker of these cells.

### Example 18

This example compares the gene expression profiles of human small airway epithelium and alveolar macrophages in response to cigarette smoking.

Rationale: Both the small airway epithelium (SAE) and alveolar macrophages are exposed to the oxidant stress of cigarette smoking, with the epithelium becoming dysfunctional, while alveolar macrophages become activated, but not diseased. In this context, the expression level of oxidant-related genes in paired samples of SAE and alveolar macrophages in healthy nonsmokers and smokers was analyzed. It was hypothesized that, in smokers, gene expression differs substantially between the two cell populations.

Methods: Affymetrix HG-U133 Plus 2.0 microarrays were used to assess expression of 154 known oxidant-related genes in both small (10th-12th order bronchi) airway epithelium and alveolar macrophages obtained by bronchoscopy from the same 15 normal nonsmokers and 29 normal smokers. Expression was defined as being present in >50% samples.

Results: Of the 154 oxidant-related genes surveyed, 113 (73%) genes were expressed in SAE and 93 (60%) genes in alveolar macrophages. However, in nonsmokers, the majority (69%) were expressed at higher levels (>1.5-fold) in alveolar macrophages than small airway epithelium (p<0.05). When assessing smoking responsiveness (>1.5-fold change, p<0.05), many (n=27) genes were identified in the epithelium, but less (n=20) in alveolar macrophages, especially in xenobiotic and redox balance categories.

Conclusions: While most oxidant-related genes are expressed at higher levels in alveolar macrophages than SAE, the epithelium is more responsive to smoking than alveolar macrophages from the same individuals. Thus, cells with an identical genome exposed to the same oxidant stress react differentially, with the alveolar macrophages more subdued than the SAE, consistent with the observation that SAE is vulnerable and alveolar macrophages are not diseased in chronic cigarette smokers.

### Example 19

This example examines the effect of gender on the response of small airway epithelium to cigarette smoking.

Rationale: Increasing data suggests women are more susceptible to cigarette smoke than men. Studies comparing female and male smokers with COPD suggest that females have a faster decline in FEV1, greater airway responsiveness, more respiratory symptoms, and increased rate of hospitalizations. In 2000, for the first time, more women died from COPD than men. In this context, it was hypothesized that females have a different small airway epithelial gene expression response to smoking than males.

Methods: Small airway epithelium (10th-12th generation) was obtained via bronchoscopic brushing in healthy smokers [n=26; 13 males (M) and 13 females (F), matched for age and pack-yr] and in control healthy non-smokers (n=20; 10 M and 10 F, matched for age). Gene expression was assessed with Affymetrix HG-U133 Plus 2.0 microarrays and 2-way ANOVA analysis was performed to evaluate the interaction between sex and smoking.

Results: 452 probe sets had an interactive p value of <0.01, indicating a differential response to smoking in men and women, including 13 with p<0.001 representing 10 genes. The most highly represented functional categories were transcription (15.7%), signal transduction (9%), and metabolism (7%). The list also included genes relevant to oxidant response to cigarette smoke (e.g., selenoprotein T, oxidation resistance 1) and immune response (e.g., interleukin 6 signal transducer), all of which were down-regulated in female smokers and unchanged in male smokers.

Conclusions: These observations suggest female smokers have significantly different biologic responses than males of the small airway epithelium to the stress of smoking. This has implications for the growing body of clinical data suggesting that smoking has a greater impact in females than males.

### Example 20

This example describes the of modulation of matrix metalloproteinase 1 gene expression in small airway epithelium of healthy smokers and nonsmokers.

Rationale: Variable expression of matrix metalloproteinase 1 (MMP1) in airway epithelium and literature implicating MMP1 in the etiology of COPD led to the hypothesis that genetic variation may influence gene expression levels of MMP1 in the small airway epithelium (SAE), the earliest site of involvement in COPD.

Methods: Affymetrix HG-U133 Plus 2.0 microarrays were used to assess MMP1 gene expression in the small (10^{th} to 12th order bronchi) airway epithelium obtained by bronchoscopy from 26 healthy nonsmokers and 36 healthy normal smokers. The Affymetrix Human SNP array 5.0 assessed single nucleotide polymorphisms (SNPs) within 100 kbp of the MMP1 gene and the correlation of SAE MMP1 gene expression with genotype was examined using PLINK software. For the high and low expressors, the MMP1 promoter was sequenced.

Results: There was a significant correlation of the levels of small airway MMP1 epithelial expression with SNP rs470215. The CC genotype was associated with a mean relative expression level of 0.27±0.07, compared to an expression level of 1.12±0.13 with the TT genotype (p<6×10−7). The MMP1 expression level was not influenced by smoking status (p>0.5) or genetic ancestry (p>0.8 by ANOVA). SNP rs470215 was not in linkage disequilibrium with the known COPD-associated promoter SNP rs1799750, nor did the two SNPs genotypes correlate with each other. Thus, rs470215 represents a new linkage that modulates levels of small airway epithelial MMP1.

Conclusions: MMP1 gene expression in the SAE is genetically determined by SNP rs470215 or by a linked SNP. This genetic variation in the modulation of MMP1 expression may contribute to the genetic variation underlying the risk for COPD.

### Example 21

This example determines the expression of genes encoding proteases an antiproteases in airway epithelium.

Rationale: Because only a sub-group of chronic smokers develop COPD, it was hypothesized that there are genetically determined differences in the gene expression profile in airway epithelium that may impact epithelial protective functions. For example, genes encoding proteases and antiproteases are a group of genes critical for protection against smoking-related lung disease.

Methods: Gene expression levels in small airway epithelium, obtained by fiberoptic bronchoscopy and brushing, were assessed with Affymetrix HG-U133 Plus 2.0 arrays in 90 subjects. Blood was obtained on the same subjects and the genotype for 441,000 SNPs was determined with the Affymetrix 5.0 SNP chip. The correlation of cis genotype (all SNPs within 25 kb of the gene) was determined for all genes expressed in small airway epithelium.

Results: A total of 512 probesets representing 151 named genes showed significant (Bionferroni corrected p value<0.05) correlation of expression level with at least one SNP within 25 kb of the gene. For 60 of these genes (40%), there were >3 adjacent SNPs that correlated with expression level. Functional categorization of the significant genes was notable for 12 genes from protease/anti-protease group including: three SERPINS (SERPINA6, SERPINB5 and SERPINB11), four ubiquitin-associated genes (UBE1L2, UBE2, USP36, USP7), tripeptidyl peptidase II (TPP2), leukocyte-derived arginine aminopeptidase (LRAP), cathepsin S (CTSS), caspase 8 (CASP8), and type 1 TNFR shedding aminopeptidase regulator (ARTS-1).

Conclusion: There are strong effects of local genotype on the expression level of multiple protease/antiprotease-related genes in small airway epithelium. These variations may contribute to the effectiveness of the protection provided by the small airway epithelium against smoking-related COPD.

### Example 22

This example compares gene expression in upper versus lower lobe small airway epithelium in patients with early emphysema and predominant upper lobe emphysema.

Rationale: Emphysema associated with cigarette smoking has a prominent upper lobe distribution. Based on the knowledge that early emphysema is associated with abnormalities of the small airways, it was hypothesized that in smokers with early emphysema, the upper lobe small airway epithelium (SAE) gene expression differs from that of the lower lobes, representing a more advanced transcriptional signature in the progression of COPD.

Methods: SAE was obtained from paired samples from the right upper (RUL) and right lower (RLL) lobes by bronchoscopy with brushings from 11 individuals (7 males, 4 females, age 51±7, all smokers, 29±14 pack-yr) with an early COPD phenotype (normal FEV1/FVC, reduced DLCO, emphysema on CT scan). Gene expression was analyzed with Affymetrix HG-U133 Plus 2.0 microarrays; differentially expressed genes (RUL vs RLL) were selected as up- or down-regulated>1.5-fold, p<0.05.

Results: Comparing the SAE of RUL to RLL, 226 genes were differentially expressed in categories linked to COPD pathogenesis, including xenobiotics-antioxidants, cell growth, cell adhesion, immune response and apoptosis. For example, the theoredoxin reductase 2 gene, involved in intracellular redox, the mediator of DNA damage checkpoint 1 gene, the pro-apoptotic genes BCL2-interacting protein and BCL2-associated X protein, and the anti-apoptotic genes BCL2 and APAF1 interacting protein were all down-regulated in RUL compared to the RLL, whereas the pro-apoptotic gene p18 was up-regulated in the RUL (all p<0.05).

Conclusion: Smokers with early COPD and predominant upper lobe emphysema have a different transcriptional signature of SAE in the upper versus lower lobes. These genes may represent markers of the pathways involved in the early progression of COPD.

### Example 23

This example examines the effect of age on small airway epithelium gene expression.

Rationale: Lung function and airway particle clearance decrease with aging, in contrast to the increase prevalence of respiratory symptoms. Since the SAE is often a target of lung diseases, it was hypothesized that there are SAE gene expression differences in young versus older individuals.

Methods: Small airway (10th-12th generation) epithelium was obtained via bronchoscopy and brushing in 29 healthy nonsmokers (ages 22 to 73 yr; younger group<40 yr, n=14; older group>40 yr, n=15), matched for gender and ancestry. Affymetrix HG-U133 Plus 2.0 microarrays were used to assess global gene expression, with differentially expressed genes defined by a 1.5-fold up- or down-regulation with p<0.01.

Results: 177 gene probe sets were differentially expressed in the SAE of older versus younger healthy nonsmokers. These probe sets comprised different categories including genes related to cell cycle regulation, signal transduction, antigen processing and presentation, and immune-related genes. For example the cyclin D1, the transducer of ERBB2, and the RAP1 interacting factor homolog genes, all involved in cell cycle progression, were up-regulated in older healthy individuals (p<0.001). Similarly, the β2 microglobulin gene, important in antigen processing and presentation, was up-regulated in older individuals (p<0.0001).

Conclusion: The data suggests that in healthy nonsmokers, the effect of aging on the SAE is reflected by changes in gene expression. These findings are relevant to understanding the molecular changes linked to the normal physiologic decline in lung function during aging in the absence of lung disease, and may help in the identification of potential pathways linked to the accelerated decline in lung function in the presence of lung disease in the older population.

### Example 24

This example compares gene expression in the small airway epithelium of symptomatic versus asymptomatic smokers with normal lung function.

Rationale: The presence of respiratory symptoms in smokers with normal lung function is associated with a more rapid decline in FEV1, development of COPD, and a higher risk of mortality compared with healthy asymptomatic smokers. On this basis, it was hypothesized that the small airway epithelium of symptomatic smokers with normal lung function has different gene expression profiles compared to that of asymptomatic smokers.

Methods: Small airway (10th-12th generation) epithelium was obtained via fiberoptic bronchoscopy and brushing of active smokers (10 symptomatic and 21 asymptomatic; normal lung function) matched by age, gender, ancestry and smoking history. Symptoms were defined by the presence of cough and/or sputum most days of the week. Affymetrix HG-U133 Plus 2.0 microarrays were used to assess global gene expression, and differentially expressed genes defined by p call>20% and 1.5-fold up- or down-regulation with p<0.01.

Results: Comparison of symptomatic versus asymptomatic smokers revealed 127 probe sets differentially expressed in small airway epithelium in different functional categories, including genes related to apoptosis, cell cycle regulation, signal transduction, proteolysis and immune response. For example, the protease inhibitor SERPINB4 is downregulated 1.7-fold and the oxidant-related aldo-keto reductase 1A1 is up-regulated 1.5 fold in symptomatic smokers relative to healthy smokers.

Conclusions: The small airway epithelium of symptomatic smokers demonstrates differential gene expression compared with asymptomatic smokers in categories that may be linked to the pathogenesis of respiratory symptoms and eventual development of COPD. These results are relevant to the biologic mechanisms involved in the variable response of the human airway epithelium to smoking.

### Example 25

This example demonstrates the in vivo transcription response of alveolar macrophages among healthy cigarette smokers.

Rationale: Alveolar macrophages (AM) are central in the defense and maintenance of normal lung structure and function in response to the stress of cigarette smoke. Since only a minority of smokers develop lung disease, it was hypothesized that AM of healthy smokers would show disparate patterns of gene expression in response to smoking.

Methods: AM from bronchoalveolar lavage of nonsmokers (n=18) and normal smokers (n=33, 26±17 pk-yr) were assessed with Affymetrix HG-U133 Plus 2.0 microarrays to identify differentially expressed genes (fold-change>1.5, p<0.01, Benjamini-Hochberg correction). Log 2 transformed data was used to define normal for each probe set as within two standard deviations of the nonsmokers mean in the direction of the smoking-induced change. An AM gene expression index (I_{AM}) was calculated as % probe sets abnormally expressed.

Results: A large number of probe sets (121 representing 95 genes) were differentially expressed in smokers versus nonsmokers. The majority (80%) were down-regulated, including genes in the categories of immune response, signal transduction and transport. The I_{AM} clearly separated nonsmokers from smokers (p<0.001), but there was no correlation with age, sex, ethnicity, or pk-yr (p>0.2). Compared to low index smokers, high index smokers showed greater down-regulation in immune response genes TNFAIP6, IF127, C1S (p<0.01) but no significant difference in chemokine ligands, CCL5, CXCL11, and CXCL9 (p>0.1).

Conclusions: The AM response to the stress of cigarette smoking is variable among the population of healthy smokers, with a 5-fold disparity between the high I_{AM} vs low I_{AM} smokers. This index highlights the extreme variability of gene expression within different categories among smokers and is consistent with the evidence that only a subset of smokers develop lung disease.

### Example 26

This example compares the expression of oxidant-related genes in the small versus large airway epithelium.

Rationale: Cigarette smoke provides a significant oxidant stress to the airway epithelium, resulting in airway disorder and dysfunction. Although the epithelium of both large and small airways are exposed to the same stress, it is the small airways that are the initial site of abnormalities in smokers. On this basis, it was hypothesize that there are differences in gene expression in oxidant-related genes in the small airways of smokers compared to the large airways of the same individual.

Methods: Paired samples of bronchial brushings were obtained from the large and small airways of nonsmokers (n=19) and healthy smokers (n=33). RNA was assessed by Affymetrix HG-U133 Plus 2.0 microarrays, focusing on 154 oxidant-related genes compiled from the literature. Differentially expressed genes were defined as those with a fold-change>1.5 and p<0.05 (paired t test).

Results: In nonsmokers, 85% of the 154 oxidant-related genes were expressed in both sites, but 12% were expressed at significantly different levels, with 8% downregulated in the small compared to the large airway epithelium. In smokers, 83% of the 154 oxidant-related genes were expressed in both sites, but 18% were expressed at different levels, with 13% downregulated in the small versus large airway epithelium. Examples of differentially expressed genes included glutathione S transferase A4 (GSTA4) which was expressed at 1.9-fold higher levels in large versus small airway. In contrast, glutathione S transferase A3 was expressed at a 1.5-fold lower level in the large airways.

Conclusions: Oxidant gene expression in the small versus the large airway of the same individual is different and these differences are more pronounced in smokers, with the small airways demonstrating lower levels of expression. This observation has relevance to the clinical observation that the small airways are more vulnerable to the effects of cigarette smoke.

### Example 27

This example describes the establishment of quality control criteria to minimize experimental variability in microarray assessment of human airway epithelial gene expression.

Rationale: Microarray technology provides a powerful tool for identifying gene expression profiles of airway epithelium that lend insight into the pathogenesis of human airway disorders. The focus of this example was to establish rigorous quality control (QC) parameters that ensure the microarray data reflects genuine biological changes and is not confounded by experimental artifact.

Methods: Pre- and post-chip QC criteria were established including: (1) RNA quality, assessed by RNA Integrity Number (RIN)>7.0, Agilent Bioanalyzer software; (2) cRNA transcript integrity, assessed by signal intensity ratio<3.0 of GAPDH 3 to 5 probe sets; and (3) the multi-chip normalization Scaling Factor, with all samples within mean 2 standard deviations. As a test of these criteria, small airway epithelium was collected via fiberoptic bronchoscopy of 43 healthy smokers and 28 healthy nonsmokers. Data quality was confirmed by Affymetrix HGU133 2.0 Plus microarray derived expression levels for 100 housekeeping genes.

Results: Of the smokers, 93% passed the RIN criterion, compared to 96% nonsmokers. No samples failed the GAPDH 3/5 ratio criterion. For the Scaling Factor criterion, 95% smokers and 100% nonsmokers passed. Across the data set of all samples passing the tripartite QC criteria (n=67), the coefficients of variation of housekeeping gene expression values were 25.6 3.7%, all below the recommended 40% level.

Conclusions: Using the QC criteria, smokers exhibit a trend towards a greater number of failures than non-smokers, but retain an acceptable 93% success rate. For samples passing this rigorous, pass/fail criteria, expression levels for housekeeping genes exhibit sample-independent stability and an acceptable level of variance across smoking status and technical factors related to sample processing.

### Example 28

This example compares oxidant-related gene expression in tracheal and bronchial epithelium in healthy smokers.

Rationale: Smoking is the major risk factor for COPD with an estimated 1014 free radicals per puff placing the airway epithelium under an enormous oxidant stress. Based on the knowledge that there are significant changes to oxidant-related gene expression in the bronchial epithelium, it was hypothesized that the tracheal epithelium can serve as a canary for early smoking-induced changes that accurately reflects the profile of oxidant-responsive genes expressed in the large airway epithelium.

Methods: Using fiberoptic bronchoscopy and brushing to obtain airway epithelium, Affymetrix HG-U133 Plus 2.0 microarrays were used to assess 335 oxidant-related probesets in tracheal epithelium (26 healthy smokers, 22 healthy non-smokers) and large airway epithelium (2nd-3rd order; 39 healthy smokers, 23 healthy non-smokers). The differentially expressed oxidant-related probesets in each location were defined as present 20%, fold change>1.5 smokers versus non-smokers, p<0.01. Common differentially expressed probesets were evaluated by analysis of covariance (ANCOVA) to assess for the effect of smoking and location.

Results: Of the 335 oxidant-related probe sets, 43 (13%) were significantly modified by smoking in the trachea and 39 (12%) in the large airway epithelium. Of these, 33 probe sets were overlapping (p<0.001), i.e., the trachea serves as a valid canary that identifies the majority of genes responsive to smoking in the large airways.

Conclusion: The changes in oxidant-related gene expression in tracheal epithelium modified by smoking are representative of the changes that occur in the large airway epithelium, supporting the concept of using tracheal epithelium as a source of biomarkers for early diagnosis and intervention.

### Example 29

This example demonstrates a method for determining the index of SAE gene expression (I_{SAE}), and correlating the I_{SAE} to an individual's risk of developing COPD.

Affymetrix HG-U133 Plus 2.0 microarrays were used to assess gene expression patterns of 34 healthy non-smokers, 41 healthy smokers, and 20 smokers with COPD according to the following method.

### Sampling Airway Epithelium

Fiberoptic bronchoscopy was used to collect small airway epithelial cells by brushing the epithelium. After mild sedation with meperidine and midazolam, and routine anesthesia of the vocal cords and bronchial airways with topical lidocaine, a fiberoptic bronchoscope (Pentax, EB-1530T3) was positioned proximal to the opening of a desired lobar bronchus. A 2.0 mm diameter brush was advanced approximately 7 to 10 cm distally from the 3rd order bronchial branching and the distal end of the brush was wedged at about the 10th to 12th generation branching of the right lower lobe. Small airway epithelial cells were collected by gently gliding the brush back and forth on the epithelium 5 to 10 times in 8 to 10 different locations in the same general area. Airway epithelial cells were detached from the brush by flicking into 5 ml of ice-cold LHC8 medium (GIBCO, Grand Island, N.Y.). An aliquot of 0.5 ml was used for differential cell count (typically 2×10⁴ cells per slide). The remainder (4.5 ml) was processed immediately for RNA extraction. The total number of cells recovered by brushing was determined by counting on a hemocytometer. To quantify the percentage of epithelial and inflammatory cells and the proportions of basal, ciliated, secretory, and undifferentiated cells recovered, cells were prepared by centrifugation (Cytospin 11, Shandon Instruments, Pittsburgh, Pa.) and stained with Diff-Quik (Baxter Healthcare, Miami, Fla.), and differential cell counts were performed by experienced observers.

### RNA and Microarray Processing

The HG-U133 Plus 2.0 array (Affymetrix, Santa Clara, Calif.), which includes probes representing approximately 47,000 full-length human genes, was used to evaluate gene expression. Total RNA was extracted using a modified version of the TRIzol® method (InVitrogen, Carlsbad, Calif.), in which RNA is purified directly from the aqueous phase by Rneasy purification (Rneasy MinElute RNA purification kit, Qiagen, Valencia, Calif.). RNA samples were stored in RNA Secure™ (Ambion, Austin, Tex.) at −80° C. RNA integrity was determined by running an aliquot of each RNA sample on an Agilent Bioanalyzer (Agilent Technologies, Palo Alto, Calif.). The concentration was determined using a NanoDrop ND-1000 spectrophotometer (NanoDrop Technologies, Wilmington, Del.). RNA samples accepted for further processing met three quality control criteria: (1) A260/A280 ratio between 1.7 and 2.3; (2) RNA concentration>0.2 μg/ml; and (3) Agilent electropherogram displaying two distinct peaks corresponding to the 28S and 18S ribosomal RNA bands at a ratio of >0.5 with minimal or no degradation. Double stranded cDNA was synthesized from 3 μg total RNA using the GeneChip® One-Cycle cDNA Synthesis Kit, followed by cleanup with GeneChip® Sample Cleanup Module, in vitro transcription (IVT) reaction using the GeneChip® IVT Labeling Kit, and cleanup and quantification of the biotin-labeled cDNA yield by spectrophotometry. All kits were from Affymetrix (Santa Clara, Calif.). Hybridizations to test chips and to the HG-U133 Plus 2.0 microarray were performed according to Affymetrix protocols, processed by the Affymetrix fluidics station, and scanned with an Affymetrix Gene Array Scanner 2500. Overall microarray quality was verified by the following criteria: (1) RNA Integrity Number (RIN)>7.0; (2) 3′/5′ ratio for GAPDH<3; (3) Scaling factor range no more than ±2.5 standard deviations from the mean for all microarrays; and (4) Expression level for all 100 housekeeping genes (as defined by Affymetrix) with a coefficient of variation of <40%.

### Microarray Data Analysis

Microarray data were processed using the MAS5 algorithm (Affymetrix Microarray Suite Version 5 software), which takes into account the perfect match and mismatch probes. MAS5-processed data were normalized using GeneSpring by setting measurements<0.01 to 0.01 and by normalizing per chip to the median expression value on the array. To make the index calculation applicable to independent data sets and to subsequently collected samples, data were not normalized per gene to the median expression value across arrays. Genes that were significantly modified by smoking were selected according to the following criteria: (1) P call of “Present” in≧20% of samples; (2) magnitude of fold change in average expression value for healthy smokers versus nonsmokers≧1.5; (3) p<0.01 with a Benjamini-Hochberg correction to limit the false positive rate. Functional annotation was carried out using the NetAffx Analysis Center (Affymetrix, Santa Clara, Calif.) to retrieve the Gene Ontology (GO) annotations from the National Center for Biotechnology (NCBI) databases. For genes without GO annotations, other public databases were searched (e.g., Human Protein Reference Database, Kyoto Encyclopedia of Genes and Genomes, and PubMed). Hierarchical clustering was carried out for the significantly changed genes using the MAS5-analyzed data with the Spearman correlation as similarity measure and the complete linkage clustering algorithm using GeneSpring software.

### Index of Airway Gene Expression

The gene expression index for small airway epithelium (I_{SAE}) was constructed as follows. A list of 619 probe sets significantly differentially expressed in smokers versus nonsmokers was identified. Those probe sets represented 384 known genes, which were represented by 486 probe sets, and only those probe sets were used for subsequent analysis. Expression values for these probe sets were log₂ transformed. For each probe set, a mean and standard deviation were calculated from the values in nonsmokers, and the normal range was defined as within two standard deviations of the mean, in the direction of the smoking-induced change (i.e., for smoking-suppressed genes, the threshold for normal equals the mean minus two standard deviations, and for smoking-induced genes the threshold for normal equals the mean plus two standard deviations). In order to avoid biasing I_{SAE} in favor of genes represented by multiple probe sets, for each gene, the proportionality factor “pf” represented the number of probe sets for that gene. For each probe set, each individual's expression was compared to the normal range and given a score of “1/pf” if the expression value was abnormal and a “0” otherwise. The proportionality factor “pf” served to make the maximum attainable score per gene equal to 1, i.e. if 4 probe sets represented a particular gene, an abnormal value for any one probe set would earn a score of 0.25, and abnormal expression in all four would obtain a total score of 1. These scores were summed for each individual and divided by 384, i.e., the number of genes in the index. For the small airway epithelium, therefore,

$${I}_{S A E} ( \% ) = {\sum }_{n = 1}^{573} cEn$$

where E1 is an index for probe set 1 whose value is 1/pf if the expression level is >2 SD above or below that of healthy smokers or 0 otherwise; E2 is 1/pf or 0 for probe set 2, etc., and the constant (c=100/384) serves to make this index equal to the percent of the 384 genes that are outliers.

To facilitate comparisons between different groups of smokers, the smokers were divided into quartiles based on I_{SAE} values. The smokers with index values falling into the first and second quartiles were deemed “low responders” and those in the third and fourth quartiles were deemed “high responders” to the stress of smoking.

### Validation of ISAE

To ensure that the I_{SAE} was not confounded by parameters other than the inherent responses of the individual smokers, the I_{SAE} was assessed in the context of analysis-related parameters (RNA integrity number (RIN); 5′ to 3′ ratio for glyceraldehyde 3-phosphate dehydrogenase (GAPDH, a housekeeping gene); and chip scaling factor); demographic-related parameters (age, gender, and ancestry), and smoking-related parameters (pack-yr smoked, urine cotinine, urine nicotine, and venous carboxyhemoglobin levels). For continuous variables (e.g., RIN, 5′ to 3′ ratio for GAPDH, scaling factor, pack-yr, age, urine cotinine, urine nicotine, and venous carboxyhemoglobin) correlation coefficients were calculated and expressed as r2. For discrete variables (e.g., gender, ancestry) ANOVA was used to evaluate for significant differences among the groups.

### Results

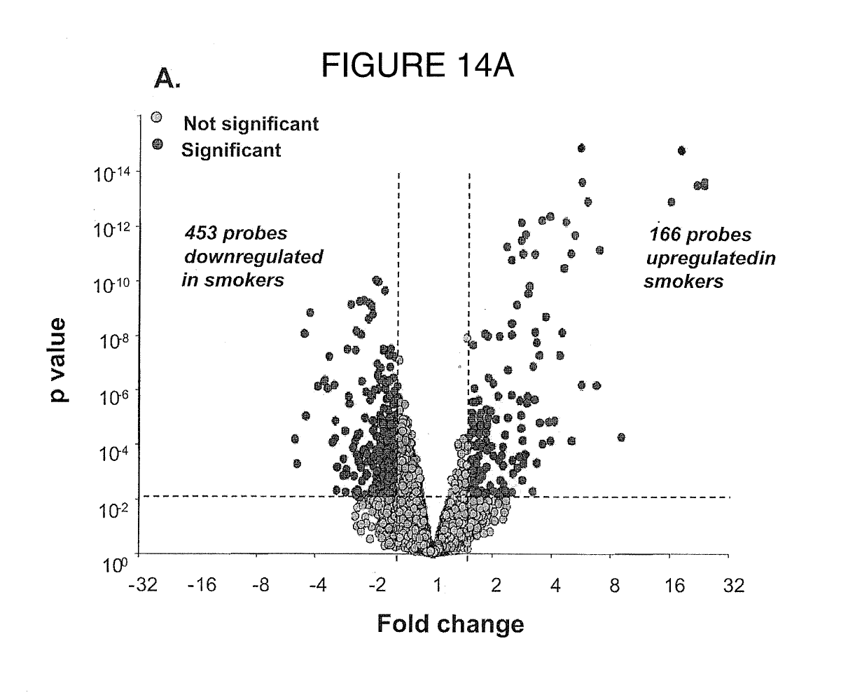

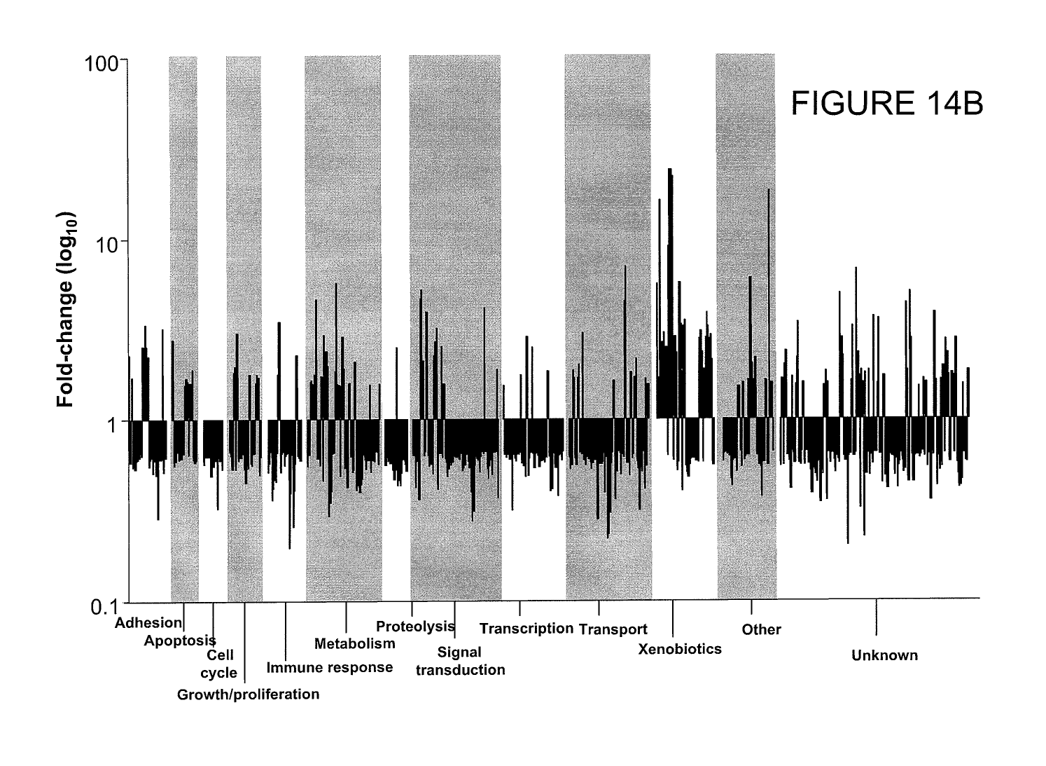

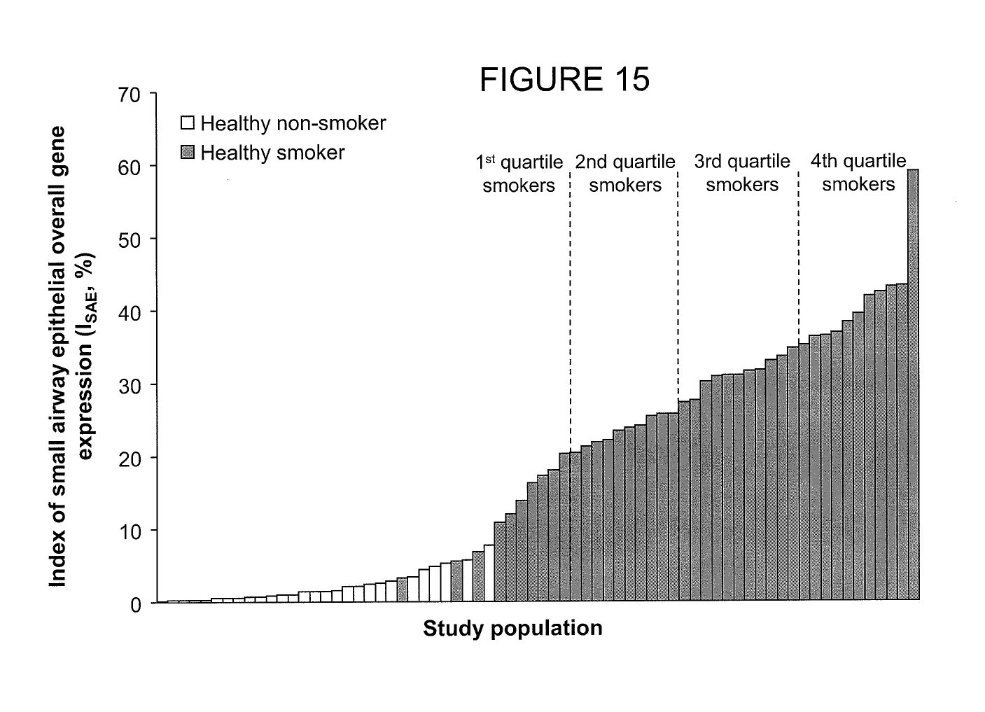

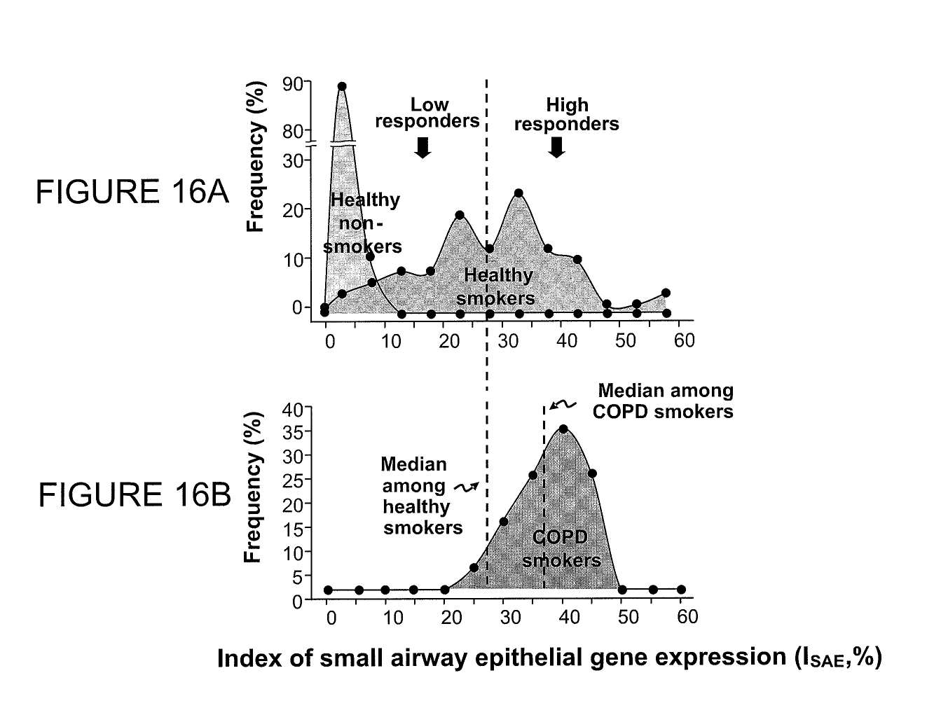

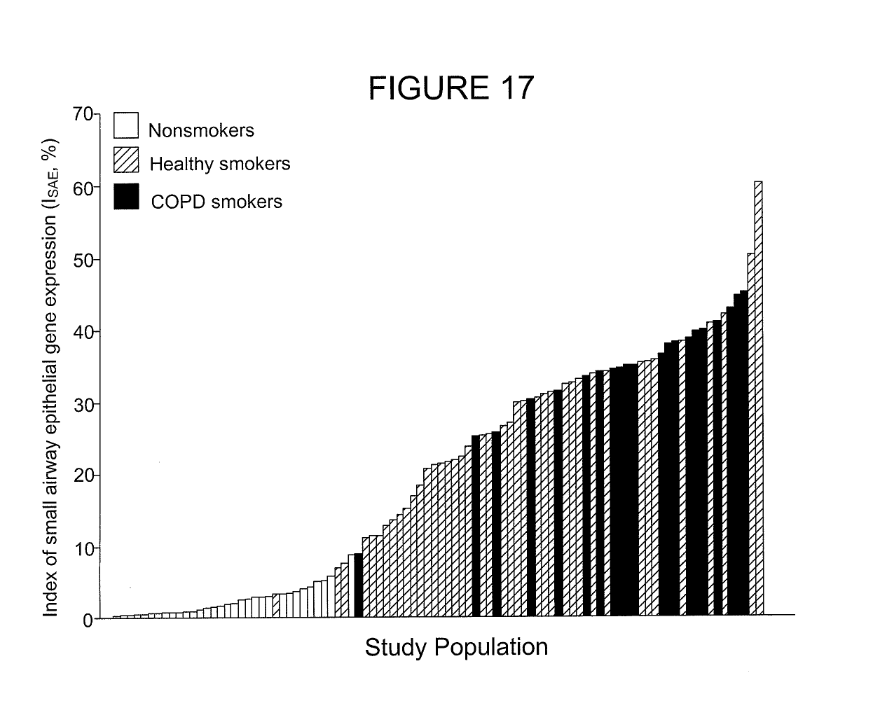

For healthy nonsmokers, the I_{SAE} ranged from 0% to 8.8% (median 1.6%). In contrast, smokers had wide variation in the I_{SAE}, from 3.3% to 60.3% (median 25.5%) (See FIGS. 14A-B, and 15). When the I_{SAE} was assessed in smokers with COPD (n=20, GOLD I-III), the I_{SAE} grouped with the healthy smoker high responders (smokers with COPD median 35.1%) (See FIGS. 16A-B and 17). The finding of higher I_{SAE} in individuals with COPD versus normal smokers suggests that smokers with a greater smoking response may be at higher risk for COPD.

### Example 30

This example demonstrates the variability of small airway epithelium gene expression in response to cigarette smoke among healthy individuals and individuals with COPD.

Rationale: Since only 15-25% of smokers develop COPD, it was hypothesized that the SAE gene expression response of individual smokers varies and quantified that response using an index that captures the changes in SAE mRNA expression (I_{SAE}).

Methods: SAE (10th-12th generation) was obtained via bronchoscopic brushing of nonsmokers (n=28) and healthy smokers (n=42, 27±17 pack-yr). Affymetrix HG-U133A Plus 2.0 microarrays were used to identify differentially expressed genes (fold-change>1.5, p<0.01, Benjamini-Hochberg correction (p(BH)), see FIG. 1). Data was log 2 transformed and for each probe set, the threshold for normal was set at two standard deviations from the nonsmokers mean in the direction of the smoking-induced change. Each individual I_{SAE} was calculated as the % probe sets abnormally expressed.

Results: Assessment of SAE in nonsmokers versus smokers showed 573 probe sets were differentially expressed. For healthy nonsmokers, I_{SAE} ranged from 0.2% to 7.7%. In contrast, smokers had wide variation in I_{SAE}, from 3.3% (19/573) to 59.0% (338/573; p<0.0001 smokers vs nonsmokers). When I_{SAE} was assessed in individuals with COPD (n=21, GOLD I-III), values were significantly higher, with average I_{SAE} for COPD 36.0% versus 27.4% for healthy smokers (p<0.0004).

Conclusions: The response of small airway epithelial gene expression to the insult of smoking is variable, with some smokers showing a much higher response than others. This can be quantified using a simple metric, the I_{SAE}, which discriminates well between nonsmokers and smokers. The finding of higher I_{SAE} in individuals with COPD versus normal smokers suggests the hypothesis that smokers with a greater smoking response may be at higher risk for COPD.

### Example 31

This example demonstrates smoking-induced changes in the biologic phenotype of the human tracheal epithelium that are assessed with a rapid, office-based procedure.

Rationale: Microarray analysis of airway epithelium of healthy smokers shows up- and down-regulation of hundreds of genes. With the goal of developing a gene expression biomarker of environment-induced lung disease, an office procedure was developed to obtain tracheal epithelium without sedation, permitting identification of smoking-responsive genes and creation of an index of tracheal epithelial gene expression (I_{T}) that separates healthy nonsmokers from healthy smokers.

Methods: After topical anesthetic without sedation, tracheal epithelium was obtained by bronchoscopic brushing of healthy nonsmokers (n=15) and healthy smokers (n=10, 27±18 pack-yr) typically in <20 minutes. Affymetrix HG-U133 Plus 2.0 microarrays were used to assess gene expression differences [fold-change (FC)>1.5, p<0.05, Benjamini-Hochberg correction]. All data was log 2 transformed, and for each smoking-responsive probe set the threshold for normal was set at 2SD from the mean in nonsmokers in the direction of the smoking-induced change. For each individual, I_{T} was defined as the % probe sets abnormally expressed.

Results: 208 probe sets were differentially expressed in healthy smokers versus non-smokers, including those in categories relevant to COPD pathogenesis, such as xenobiotic metabolism/oxidant related. Examples include CYP1B1 (FC 40, p<0.03), AKR1B10 (FC 16, p<0.04), and GPX2 (FC 4.6, p<0.005). I_{T} segregated nonsmokers from smokers, with a range of 0-5.8% in nonsmokers and 13.0-77.4% in smokers (p<0.001).

Conclusion: An office procedure without sedation allows sampling of tracheal epithelium. I_{T} differentiates smokers from nonsmokers and defines individual variability to the stress of smoking. This approach may be useful to develop biomarkers to identify smokers at risk for COPD, providing an office opportunity for early diagnosis and intervention.

### Example 32

This example compares cytochrome P450 gene expression in small airway epithelium of healthy smokers and healthy nonsmokers.

Rationale: The pathogenesis of COPD and lung cancer is tightly linked to exposure to environmental chemicals. There are >1,000 chemicals in cigarette smoke and many of these are metabolized and detoxified by the cytochrome P450 (CYP) gene family, in some cases yielding more toxic compounds and carcinogens. Based on the knowledge that the earliest evidence of smoking-induced lung disease is in the small airways, CYP gene expression in the small airway epithelium (SAE) of healthy smokers and healthy non-smokers was evaluated.

Methods: SAE (10th-12th generation) was obtained via bronchoscopy and brushing of nonsmokers (n=28) and healthy smokers (n=42, 27±17 pack-yr). Affymetrix HG-U133 Plus 2.0 microarrays were used to assess expression of CYP genes, with expression defined as present in >20% of samples. Smoking related differential expression was defined as fold-change>1.5 increase/decrease and p<0.01 with Benjamini-Hochberg correction.

Results: Of the 57 CYP enzymes for which there are probesets, 34 (60%) were expressed in the SAE of nonsmokers and 35 (61%) in smokers. Cigarette smoking altered the gene expression pattern of 6 (11%) CYP genes in the SAE. Nine CYP genes were identified that were not previously recognized as being expressed. Of these nine, two were differentially expressed by smoking: CYP4F3 (2.2-fold, p<0.00004) and CYP4F11 (2.8-fold, p<0.0003). CYP4F3 inactivates and degrades leukotriene B4, a potent mediator of inflammation, whereas CYP4F11 is without a known function.

Conclusion: About 60% of known CYP genes are expressed in SAE, with 11% exhibiting modified expression with smoking. Since the SAE is the earliest site of smoking-induced lung disease, an understanding of the genetic control of the CYP enzymes is central to understanding susceptibility to these disorders.

### Example 33

This example demonstrates the impact of copy number variation polymorphisms on gene expression in small airway epithelium.

Rationale: Genome wide surveys have demonstrated the prevalence of copy number variation (CNV) polymorphisms in the human genome, with some deletions encompassing several genes. When heterozygous, deletions result in domains of the genome being haploid. It was hypothesized that CNV results in local differences in gene expression levels in the small airway epithelium (SAE).

Methods: Affymetrix HG-U133 Plus 2.0 microarrays were used to survey expression level of all genes in the SAE obtained by bronchoscopy and brushing of 112 individuals. The genotypes of the same 112 subjects were determined by Affymetrix Human SNP array 5.0 chips. Focusing on large deletions in CNV databases, the expression level of genes within those deletions was assessed.

Results: One subject had a deletion in chromosome 1 near nucleotide 144,000,000. For the 18 expression probe sets within the deletion, the SAE expression level put this subject in the bottom 25th percentile, but for the 23 probe sets representing adjacent genes, this subject ranked on average at the 50th percentile in expression level (p<0.005, signed rank test). Similarly, a subject with deletion in the SERPINB gene cluster on chromosome 18 had a mean SAE expression level in the bottom 34th percentile for genes within the deletion compared to the 62nd percentile for adjacent genes. By contrast, when the MMP gene cluster on chromosome 11 was investigated, 5 subjects were identified with deletions encompassing MMP10, but the SAE expression level in these subjects was no different (p>0.5) than in subjects diploid for this domain.

Conclusions: Some CNV polymorphisms can impact expression levels in small airway epithelium in vivo while others have no effect. In view of the extensive CNV polymorphism in the human population, some of these may impact the expression level of critical genes whose derangement may impact pulmonary health.

### Example 34

This example describes the effect of smoking on airway epithelium gene expression profiles among individuals of European, African, South Asian, and Arabian ancestry.

Rationale: Cigarette smoking is the major cause of airway disorders throughout the world, and it was hypothesized that the environmental stress of smoking induces a predictable airway epithelial gene expression profile, regardless of genomic variability among individuals of different ancestry.

Methods: Expression profiles of airway epithelium obtained by fiberoptic bronchoscopy were assessed in 38 smokers and 25 non-smokers using Affymetrix microarrays. K-nearest neighbor (K=5) class prediction (smoker versus non smoker) was used to obtain a gene expression predictor set from New York individuals of European and African ancestry. The predictor set was assessed by ability to identify smoking status of individuals of South Asian (Nepalese, Indian, Pakistani and Bangladeshi) and Arab (Qatari and Palestinian) ancestry residing in Qatar.

Results: The European/African ancestry predictor set, comprised of 150 probe sets with an average (SE) predictor strength of 1.02±0.02, was validated as 100% of blinded source population samples were correctly identified for smoking status. The same predictor set correctly predicted 90% of the samples from individuals of Southeast Asian and Arab ancestry.

Conclusions: Gene expression changes of the airway epithelium of smokers is so universal (i.e., smoking is such a dominant environmental stress), that an expression predictor set of 150 genes accurately identifies smokers from non-smokers among a diverse world-wide population, despite disparate ancestral origins and concomitant disparate genomes.

### Example 35

This example describes the ancestral differences in small airway epithelium in response to cigarette smoking.

Epidemiologic data suggest that Americans of African ancestry are more susceptible to cigarette smoking than those of European ancestry, with faster rates of lung function decline and increased mortality. In the context that the small airway epithelium (SAE) is the initial site of smoking-associated disease, it was hypothesized that: (1) the SAE gene expression profile of individuals of African ancestry responds differently to cigarette smoke compared to individuals of European ancestry; and (2) genome-wide SNP genotyping will reveal cis-acting single nucleotide polymorphisms (SNPs) correlated with expression of differentially responsive genes. Gene expression levels in SAE were assessed with Affymetrix HG-U133A Plus 2.0 arrays in 24 healthy smokers (14 of African and 10 of European ancestry) and 18 healthy non-smokers (10 of African and 8 of European ancestry). Smoking-responsive genes in each ancestral group were independently identified as significant with a fold-change>2 and a p value<0.01 in smokers compared to non-smokers. Smokers of European ancestry showed a greater number of smoking-responsive genes (n=356 genes) than smokers of African ancestry (n=188 genes) and, in general, a greater magnitude of differential expression between smokers and non-smokers. For example, xenobiotic metabolism genes were up-regulated in smokers of both groups, but cytochrome P450 1A1 and 1B1 were upregulated 16 and 20-fold, respectively in smokers of African ancestry, while the same genes were upregulated 40 and 150-fold, respectively, in smokers of European ancestry. Genome-wide SNP profiles were obtained on genomic DNA from blood samples from a large cohort of individuals using Affymetrix 5.0 SNP arrays. Significant associations of SNPs within 25,000 base pairs of many of the genes that were differentially responsive to smoking in the two ancestral groups were identified using a likelihood ratio test.

### Example 36

This example describes the transcriptional pattern of M2-polarized alveolar macrophages in healthy smokers.

Rationale: Depending on the microenvironment, macrophages undergo distinct activation programs acquiring either M1- or M2-polarized phenotypes associated with inflammation (M1) or tissue remodeling (M2). Given that cigarette smoking leads to development of both lung inflammation and remodeling, it was hypothesized that alveolar macrophages (AM) of smokers exhibit an altered program of M1-M2 polarization.

Methods: Transcriptional profiling of AM obtained by bronchoalveolar lavage from 18 healthy nonsmokers and 33 healthy smokers was carried out using Affymetrix HG-U133 Plus 2.0 microarrays (P call>50%, p value<0.05, Benjamini-Hochberg correction).

Results: Compared to AM of nonsmokers, AM of smokers exhibited suppressed expression of many M1 genes, including those encoding C-X-C chemokine ligands 11 (p<0.01) and 9 (p<0.01), C-C chemokine ligands 4 (p<0.05) and 5 (p<0.005), IL-1 (p<0.02). Smoking was also associated with decreased expression of antimicrobial genes (indoleamine 2,3-dioxygenase, p<0.02; phospholipase A2, p<0.02, and cathelicidin LL-37, p<0.03). While no M1 genes were expressed in AM of smokers higher than in nonsmokers, there were a number of changes in gene expression typical for M2-polarization including increased expression of extracellular matrix regulator stabilin-1 (p<0.03) and CD36 (p<0.03). Other genes with tissue remodeling potential (matrix metallopeptidase 2, p<0.003; serine peptidase HtrA1, p<0.0003) were also upregulated in AM of smokers.

Conclusions: AM of healthy smokers display a skewed expression profile with a substantial depression of M1-typical inflammatory and host defense genes and induction of an unusual pattern of M2-polarization accompanied by increased expression of genes implicated in tissue remodeling. It is possible that early changes in the lung associated with smoking may develop in an inflammation-independent manner due to reprogramming of AM towards M2-polarized macrophages.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of determining the likelihood that a smoker will or will not develop chronic obstructive pulmonary disease (COPD) comprising:
(a) providing a sample obtained from a smoker;
(b) analyzing the sample to determine an expression pattern of one or more biomarkers associated with COPD; and
(c) comparing the expression pattern determined from the sample with a standard expression pattern to determine the likelihood that the smoker will or will not develop COPD.

2. The method of claim 1 wherein one or more of the biomarkers are selected from the group consisting of genes set forth in FIG. 1.

3. The method of claim 1 wherein one or more of the biomarkers are selected from the group consisting of CCL2, MSRI, CD36, CSF1, LCN2, MMP2, A2M, PDG, RXRB, LAMA2, HSPA2, SSP1, CCR5, FCN1, MHC2TA, IFITM3, HTN1, MX2, IFITM3, C1R, ITGAE, COL6A2, ALCAM, VCL, ICAM3, P2RX7, RAP2A, PDE3B, RPS6KA1, MAPKAP1, RRAD, KIT, PHF16, PTPN3, ADAM1O, IDE, SERPINB5, LIPA, LAMP1, FUCA1, TMF1, PBX3, HES1, SNAPC1, ZNF135, IDH1, CDA, PHGD, RNASEL, SNTB1, FABP3, SULT1C1, VAT1, CLCN7, UBE2B, DMD, KRT1 7, KRT7, PLTP, ASS, KIAA0368, SPAG1, MEIS4, TNNT1, HUMRIRT, NET1, BAMBI, CXADR, HUMGT1 98A, SORL1, SAH, SLC1 5A1, EML1, ERBL1, WDRLO, TNF-α, IFN-gamma, MMP-I, -9, -12, CFMCP-I, MIP-I α, CCL3, IL-8, IFN receptor 2, IL-16, MUC1, MUC15, Ser/Thr kinase 17b, Bombesin, IL-4 receptor, spondin2, TIP30, homeodomain protein kinase, CHGA, Pirin, AZGP-I, Mucin 5AC, MERTK, Di 1 1, Hes1, Hes2, Hes5, HeyL, Di 1 1, Jag1, ABP1, ARG2, C20orf96, C21orf128, C6orf1 18, CACNB2, CALCA, CCL17, CCL20, CHAC1, CLCA4, COL3A1, CRADD, CYS1, DNAH7, DSCAM, FCGBP, FGFR1 OP2, FKBP1A, FLJ33297, FLJ36748, FLJ43663, GBP4, GPC1, GRM1, HOXA1, HS3ST3A1, HSA9761, HTR2B, IFNA4, JUNB, KCNJ1, KIAA0565, KIAA0960, KIAA1904, LDB1, LOC130355, LOC284825, LOC388335, LOC401034, LOC641941, LOC647248, LPAAT-THETA, LRRC43, MALAT1, MARCKSL1, MGC45491, MIPOL1, MT1M, MUC5B, MYL9, NCKAP1, NEB, NPTX2, PAPPA, PCDHB5, PDCD6, PER1, PLEKHA5, PRDM1 1, PRR1 2, PRR4, RAPGEFL1, RNPS1, RP1 1-444E17.2, RRAD, RSNL2, SBEM, SDCBP2, SERPINA3, SERPINH1, SLC1 3A2, SLC2A4RG, SLC39A8, SLC6A20, STK1 7B, TACR1, TBX1, TMSB4Y, TP73L, TPRXL, TTLL1 1, USH1C, USP2, VEGFB, WNT5B, ZFHX1B, ZFP36, ZNF42, ADAM-12, ARTS-I, AAA, CASP8, CTSS, LRAP, selenoprotein T, SERPINA6, SERPINB1 1, SFTPB, SLC34A2, TMEM1, TMEM37, TPP2, UBE1L2, UBE2, USP7, USP36, IF1 27, CIS, GSTA3, GSTA4, MMP1, MMP1O, MMP14, OXR1, mediator of DNA damage checkpoint1 gene, BCL2-interacting protein, BCL2-associated X protein, BCL2, APAF1 interacting protein, pi 8, cyclin D1, transducer of ERBB2, RAP1 interacting factor homolog genes, β2 microglobulin, interleukin 6 signal transducer, aldo-keto reductase IA1, glutamate-cysteine ligase catalytic subunit, glutamate-cysteine ligase regulatory subunit, glutamate-cysteine ligase modifier subunit, chemokine ligand 2, meprin A, tenascin C, bone morphogenetic protein 4, interferon alpha-inducible proteins 27, 6, and “44-like”, glutathione peroxidase 3, NADP+mitochondrial isocitrate dehydrogenase 2, glutathione S-transferase A2, aldo-keto reductase 1C3, aldo-keto reductase IB1, fructose-bisphosphate aldolase A, cell division cycle 10 (CDC10), and cell division cycle 20 homolog B (CDC20B).

4. The method of claim 1, wherein one or more of the biomarkers are ABP1, ADH7, AJAP1, AKR1B1O, AKR1C1, AKR1 C2, AKR1C3, ALDH3A1, ANGPT1, ANPEP, AOC3, ARG2, ATP12A, ATP6V0A4, ATP6V1B1, AVPR1A, AZU1, B3GNT6, C10orf39, C10orf81, C14orf132, C20orf96, C21orf128, LOC653879, C6orf1 18, CABYR, CABYR, CACNB2, CALCA, CBR1, CBR3, CCL 17, CCL20, CEACAM5, CFB, CFD, CHAC1, CHEK1, ChGn, CHI3L1, CLCA4, CLDN1O, CNGB1, CNN3, COL3A1, CRADD, CX3CL1, CX3CL1, CXCL2, CXCL3, CYP1A1, CYP1B1, CYP4F11, CYP4F3, CYP4×1, CYS1, D2HGDH, DEPDC6, DNAH7, DRD1, DSCAM, DTNA, DUSP1, DUSP5, EGF, ELMOD1, EPB41L2, EPHB1, FAM107A, FAM38A, FBN1, FCGBP, FGFR10P2, FGFR2, FKBP1A, FLJ33297, FLJ36748, F1139051, FLJ43663, FOXA2, G6PD, GAD1, GBP4, GEM, GLRB, GPC1, GPX2, GRM1, H19, HES6, HGD, HNMT, HOXA1, HS3ST3A1, HSA9761, HSD17B2, HTR2B, IFNA4, IL27RA, IRS2, ITLN1, ITM2A, JUNB, KCNJ1, KIAA0565, KIAA0960, KIAA1904, LAMB3, LDB1, LM04, LOC130355, LOC283177, LOC283514, LOC284825, LOC388335, LOC401034, LOC440338, LOC641941, LOC647248, LPAAT-THETA, LRRC43, LTF, MALAT1, MAOB, MARCKSL1, ME1, MEF2C, MGC45491, MIPOL1, MSRB3, MT1F, MT1G, MT1H, MT1M, MUC5AC, MUC5B, MYL9, NAV3, NCKAP1, NEB, NOVA1, NOVA1, NPTX2, NQO1, NT5E, NT5E, PAPPA, PCDHB5, PCSK6, PDCD6, PEG1O, PER1, PHEX, PHLDA1, PI3, PIR, PLEKHA5, PLK2, PPAP2B, PPP1R16B, PRDM1 1, PRR12, PRR4, RAPGEFL1, RHOBTB3, RNPS1, RP1 1-444E 17.2, RRAD, RSNL2, SAA1, SAA4, SBEM, SCNN1G, SDCBP2, SEC14L3, SEMA5A, SERPINA3, SERPINB10, SERPINB3, SERPINB4, SERPING1, SERPINH1, SFRP2, SFRP2, SLAMF7, SLC13A2, SLC26A4, SLC29A1, SLC2A4RG, SLC39A8, SLC6A20, SLC7A11, SLIT2, SLITRK6, SPP1, SRPX2, SRXN1, STK17B, SULF1, SUSD2, TACR1, TBX1, TFEB, TFPI, TFPI2, TMEM118, TMEM121, TMEM16D, TMEM37, TMEM45A, TMSB4Y, TP73L, TPM2, TPRXL, TTLL1 1, TXN, UCHL1, UGT1A1O, UGT1A4, UGT1A6, USH1C, USP2, VEGFB, VEPH1, VGLL1, WDR72, WNK4, WNT5B, ZBTB 16, ZFHX1B, ZFP36, ZNF42, ZNF423, and ZNF44.

5. The method of claim 1, wherein the sample is lung tissue.

6. The method of claim 5, wherein the lung tissue is large airway epithelium.

7. The method of claim 5, wherein the lung tissue is small airway epithelium.

8. The method of claim 1, wherein the sample is selected from the group consisting of trachea tissue, nasal tissue, and blood.

9. The method of claim 8, wherein the trachea tissue is trachea airway epithelium.

10. The method of claim 8, wherein the nasal tissue is nasal epithelium.

11. The method of claim 1, wherein the method further comprises treating the smoker based on the likelihood that the smoker will develop COPD.

12. The method of claim 11, wherein the smoker is diagnosed as likely to develop COPD, and the treatment of the smoker is selected from the group consisting of gene therapy, exercise, antiinflammatories, vitamins, stem cells, monoclonal antibodies, bronchodilators, corticosteroids, smoking cessation, surgery, antibiotics, theophylline, home oxygen therapy, pulmonary rehabilitation, mucolytics, TNF antagonists, vaccination against pneumococcus, and vaccination against influenza.

13. The method of claim 11, wherein the smoker is diagnosed as likely to develop COPD, and the treatment of the smoker comprises administering a therapeutically effective amount of a substance to the smoker to down-regulate one or more biomarkers whose up-regulation led to a determination that the smoker likely would develop COPD.

14. The method of claim 11, wherein the smoker is diagnosed as likely to develop COPD, and wherein the treatment of the smoker comprises administering a therapeutically effective amount of a substance to the smoker to up-regulate one or more biomarkers whose down-regulation led to a determination that the smoker likely would develop COPD.

15. A composition comprising (a) a pharmaceutically acceptable carrier and (b) a substance which causes an expression pattern of one or more biomarkers associated with COPD that is indicative of acquiring COPD to be more similar to an expression pattern of one or more biomarkers associated with COPD that is indicative of not acquiring COPD.

16. The composition of claim 15, wherein one or more of the biomarkers are selected from the group consisting of genes set forth in FIG. 1.

17. The composition of claim 15, wherein the one or more of the biomarkers are selected from the group consisting of CCL2, MSRI, CD36, CSF1, LCN2, MMP2, A2M, PDG, RXRB, LAMA2, HSP A2, SSP1, CCR5, FCN1, MHC2TA, IFITM3, HTN1, MX2, IFITM3, C1R, ITGAE, COL6A2, ALCAM, VCL, ICAM3, P2RX7, RAP2A, PDE3B, RPS6KA1, MAPKAP1, RRAD, KIT, PHF16, PTPN3, ADAM1O, IDE, SERPINB5, LIPA, LAMP1, FUCA1, TMF1, PBX3, HES1, SNAPC1, ZNF135, IDH1, CDA, PHGD, RNASEL, SNTB1, FABP3, SULT1C1, VAT1, CLCN7, UBE2B, DMD, KRT17, KRT7, PLTP, ASS, KIAA0368, SPAG1, MEIS4, TNNT1, HUMRIRT, NET1, BAMBI, CXADR, HUMGT1 98A, SORL1, SAH, SLC1 5A1, EML1, ERBL1, WDRLO, TNF-α, IFN-gamma, MMP-I, -9, -12, CFMCP-I, MIP-Ia, CCL3, IL-8, IFN receptor 2, IL-16, MUC1, MUC15, Ser/Thr kinase 17b, Bombesin, IL-4 receptor, spondin2, TIP30, homeodomain protein kinase, CHGA, Pirin, AZGP-I, Mucin 5AC, MERTK, D1 11, Hes1, Hes2, Hes5, HeyL, D1 11, Jag1, ABP1, ARG2, C20orf96, C21orf128, Cóorf1 18, CACNB2, CALCA, CCL 17, CCL20, CHAC1, CLCA4, COL3A1, CRADD, CYS1, DNAH7, DSCAM, FCGBP, FGFR10P2, FKBP1A, FLJ33297, F1136748, FLJ43663, GBP4, GPC1, GRM1, HOXA1, HS3ST3A1, HSA9761, HTR2B, IFNA4, JUNB, KCNJ1, KIAA0565, KIAA0960, KIAA1904, LDB1, LOC130355, LOC284825, LOC388335, LOC401034, LOC641941, LOC647248, LP AAT-THETA, LRRC43, MALAT1, MARCKSL1, MGC45491, MIPOL1, MT1M, MUC5B, MYL9, NCKAP1, NEB, NPTX2, PAPPA, PCDHB5, PDCD6, PER1, PLEKHA5, PRDM1 1, PRR1 2, PRR4, RAPGEFL1, RNPS1, RP1 1-444E17.2, RRAD, RSNL2, SBEM, SDCBP2, SERPINA3, SERPINH1, SLC13 A2, SLC2A4RG, SLC39A8, SLC6A20, STK1 7B, TACR1, TBX1, TMSB4Y, TP73L, TPRXL, TTLL1 1, USH1C, USP2, VEGFB, WNT5B, ZFHX1B, ZFP36, ZNF42, ADAM-12, ARTS-I, AAA, CASP8, CTSS, LRAP, selenoprotein T, SERPINA6, SERPINB1 1, SFTPB, SLC34A2, TMEM1, TMEM37, TPP2, UBE1L2, UBE2, USP7, USP36, IF1 27, CIS, GSTA3, GSTA4, MMP1, MMP1O, MMP14, OXR1, mediator of DNA damage checkpoint1 gene, BCL2-interacting protein, BCL2-associated X protein, BCL2, APAF1 interacting protein, pi 8, cyclin D1, transducer of ERBB2, RAP1 interacting factor homolog genes, β2 microglobulin, interleukin 6 signal transducer, aldo-keto reductase IA1, glutamate-cysteine ligase catalytic subunit, glutamate-cysteine ligase regulatory subunit, glutamate-cysteine ligase modifier subunit, chemokine ligand 2, meprin A, tenascin C, bone morphogenetic protein 4, interferon alpha-inducible proteins 27, 6, and “44-like”, glutathione peroxidase 3, NADP+mitochondrial isocitrate dehydrogenase 2, glutathione S-transferase A2, aldo-keto reductase 1C3, aldo-keto reductase IB1, fructose-bisphosphate aldolase A, cell division cycle 10 (CDC1O), and cell division cycle 20 homolog B (CDC20B).

18. A method to determine the efficacy of a treatment for COPD comprising
(a) providing a sample obtained from a subject that is undergoing treatment for COPD;
(b) analyzing the sample to determine an expression pattern of one or more biomarkers associated with COPD; and
(c) comparing the expression pattern determined from the sample with a standard expression pattern to determine whether the treatment for COPD has or has not been effective.

19. The method of claim 18, wherein one or more of the biomarkers are selected from the group consisting of genes set forth in FIG. 1.

20. The method of claim 18, wherein one or more of the biomarkers are selected from the group consisting of CCL2, MSRI, CD36, CSF1, LCN2, MMP2, A2M, PDG, RXRB, LAMA2, HSPA2, SSP1, CCR5, FCN1, MHC2TA, IFITM3, HTN1, MX2, IFITM3, C1R, ITGAE, COL6A2, ALCAM, VCL, ICAM3, P2RX7, RAP2A, PDE3B, RPS6KA1, MAPKAP1, RRAD, KIT, PHF16, PTPN3, ADAM1O, IDE, SERPINB5, LIPA, LAMP1, FUCA1, TMF1, PBX3, HES1, SNAPC1, ZNF135, IDH1, CDA, PHGD, RNASEL, SNTB1, FABP3, SULT1C1, VAT1, CLCN7, UBE2B, DMD, KRT17, KRT7, PLTP, ASS, KIAA0368, SPAG1, MEIS4, TNNT1, HUMRIRT, NET1, BAMBI, CXADR, HUMGT1 98A, SORL1, SAH, SLC 15A1, EML1, ERBL1, WDRLO, TNF-α, IFN-gamma, MMP-I, -9, -12, CFMCP-I, MIP-Ia, CCL3, IL-8, IFN receptor 2, IL-16, MUC1, MUC15, Ser/Thr kinase 17b, Bombesin, IL-4 receptor, spondin2, TIP30, homeodomain protein kinase, CHGA, Pirin, AZGP-I, Mucin 5AC, MERTK, Di 1 1, Hes1, Hes2, Hes5, HeyL, D1 11, Jag1, ABP1, ARG2, C20orf96, C21orf128, Cóorf 1 18, CACNB2, CALCA, CCL17, CCL20, CHAC1, CLCA4, COL3A1, CRADD, CYS1, DNAH7, DSCAM, FCGBP, FGFR1 OP2, FKBP1A, F1133297, FLJ36748, F1143663, GBP4, GPC1, GRM1, HOXA1, HS3ST3A1, HSA9761, HTR2B, IFNA4, JUNB, KCNJ1, KI AA0565, KIAA0960, KIAA1904, LDB1, LOC130355, LOC284825, LOC388335, LOC401034, LOC641941, LOC647248, LP AAT-THETA, LRRC43, MALAT1, MARCKSL1, MGC45491, MIPOL1, MT1M, MUC5B, MYL9, NCKAP1, NEB, NPTX2, PAPPA, PCDHB5, PDCD6, PER1, PLEKHA5, PRDM1 1, PRR12, PRR4, RAPGEFL1, RNPS1, RP1 1-444E17.2, RRAD, RSNL2, SBEM, SDCBP2, SERPINA3, SERPINH1, SLC13A2, SLC2A4RG, SLC39A8, SLC6A20, STK17B, TACR1, TBX1, TMSB4Y, TP73L, TPRXL, TTLL1 1, USH1C, USP2, VEGFB, WNT5B, ZFHX1B, ZFP36, ZNF42, ADAM-12, ARTS-I, AAA, CASP8, CTSS, LRAP, selenoprotein T, SERPINA6, SERPINB1 1, SFTPB, SLC34A2, TMEM1, TMEM37, TPP2, UBE1L2, UBE2, USP7, USP36, IF127, CIS, GSTA3, GSTA4, MMP1, MMP1O, MMP14, OXR1, mediator of DNA damage checkpoint1 gene, BCL2-interacting protein, BCL2-associated X protein, BCL2, APAF1 interacting protein, pi 8, cyclin D1, transducer of ERBB2, RAP1 interacting factor homolog genes, β2 microglobulin, interleukin 6 signal transducer, aldo-keto reductase IA1, glutamate-cysteine ligase catalytic subunit, glutamate-cysteine ligase regulatory subunit, glutamate-cysteine ligase modifier subunit, chemokine ligand 2, meprin A, tenascin C, bone morphogenetic protein 4, interferon alpha-inducible proteins 27, 6, and “44-like”, glutathione peroxidase 3, NADP+mitochondrial isocitrate dehydrogenase 2, glutathione S-transferase A2, aldo-keto reductase 1C3, aldo-keto reductase IB1, fructose-bisphosphate aldolase A, cell division cycle 10 (CDC10), and cell division cycle 20 homolog B (CDC20B).

21. The method of claim 18, wherein the standard expression pattern is an expression pattern determined from a sample obtained from the subject prior to the onset of treatment for COPD.

22. The method of claim 18, wherein the standard expression pattern is an expression pattern determined from a sample obtained from the subject after the onset of treatment for COPD but before the sample of step (a) was obtained from the subject.

23. The method of claim 18, wherein the sample is lung tissue.

24. The method of claim 23, wherein the lung tissue is large airway epithelium.

25. The method of claim 18, wherein the sample is selected from the group consisting of trachea tissue, nasal tissue, and blood.

26. The method of claim 25, wherein the trachea tissue is trachea airway epithelium.

27. The method of claim 25, wherein the nasal tissue is nasal epithelium.

28. The method of claim 18, wherein the treatment is selected from gene therapy, exercise, antiinflammatories, vitamins, stem cells, monoclonal antibodies, bronchodilators, corticosteroids, smoking cessation, surgery, antibiotics, theophylline, home oxygen therapy, pulmonary rehabilitation, mucolytics, TNF antagonists, vaccination against pneumococcus, and vaccination against influenza.

29. The method of claim 28, wherein the smoker is diagnosed as likely to develop COPD, and the treatment of the smoker comprises administering a therapeutically effective amount of a substance to the smoker to down-regulate one or more biomarkers whose up-regulation led to a determination that the smoker likely would develop COPD.

30. The method of claim 28, wherein the smoker is diagnosed as likely to develop COPD, and wherein the treatment of the smoker comprises administering a therapeutically effective amount of a substance to the smoker to up-regulate one or more biomarkers whose down-regulation led to a determination that the smoker likely would develop COPD.

31. The method of claim 1, wherein the biomarkers are selected from the group consisting of ADH7, AKR1B1O, AKR1C1, AKR1 C2, AKR1C3 ALDH3A1, FOXA2, G6PD, GAD1, H19, HES6, HGD, IFNA4, Intelectin1, LTF, MUC5AC, NQO1, RRAD, RSNL2, SPP1, STK17B, and UCHL1.
